SEQUENCE LISTING (57 sequences)

SEQ ID NO: 1 - protein, 352 aa, Dengue virus
DSGCVINWKGRELKCGSGIFVTNEVHTWTEQYKFQADSPKRLSAAIGKAWEEGVCGIRSATRLENIMWKQISNELNHILLENDMKFTVVVGDVSGILAQGKKMIRPQPMEHKYSWKSWGKAKIIGADVQNTTFIIDGPNTPECPDNQRAWNIWEVEDYGFGIFTTNIWLKLRDSYTQVCDHRLMSAAIKDSKAVHADMGYWIESEKNETWKLARASFIEVKTCIWPKSHTLWSNGVLESEMIIPKIYGGPISQHNYRPGYFTQTAGPWHLGKLELDFDLCEGTTVVVDEHCGNRGPSLRTTTVTGKTIHEWCCRSCTLPPLRFKGEDGCWYGMEIRPVKEKEENLVKSMVSA

SEQ ID NO: 2 - protein, 352 aa, Dengue virus
DSGCVVSWKNKELKCGSGIFITDNVHTWTEQYKFQPESPSKLASAIQKAHEEGICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTA

SEQ ID NO: 3 - protein, 352 aa, Dengue virus
DMGCVINWKGKELKCGSGIFVTNEVHTWTEQYKFQADSPKRLATAIAGAWENGVCGIRSTTRMENLLWKQIANELNYILWENNIKLTVVVGDTLGVLEQGKRTLTPQPMELKYSWKTWGKAKIVTAETQNSSFIIDGPNTPECPSASRAWNVWEVEDYGFGVFTTNIWLKLREVYTQLCDHRLMSAAVKDERAVHADMGYWIESQKNGSWKLEKASLIEVKTCTWPKSHTLWTNGVLESDMIIPKSLAGPISQHNYRPGYHTQTAGPWHLGKLELDFNYCEGTTVVITESCGTRGPSLRTTTVSGKLIHEWCCRSCTLPPLRYMGEDGCWYGMEIRPISEKEENMVKSLVSA

SEQ ID NO: 4 - protein, 352 aa, Dengue virus
DMGCVASWSGKELKCGSGIFVVDNVHTWTEQYKFQPESPARLASAILNAHKDGVCGIRSTTRLENVMWKQITNELNYVLWEGGHDLTVVAGDVKGVLTKGKRALTPPVSDLKYSWKTWGKAKIFTPEARNSTFLIDGPDTSECPNERRAWNSLEVEDYGFGMFTTNIWMKFREGSSEVCDHRLMSAAIKDQKAVHADMGYWIESSKNQTWQIEKASLIEVKTCLWPKTHTLWSNGVLESQMLIPKSYAGPFSQHNYRQGYATQTVGPWHLGKLEIDFGECPGTTVTIQEDCDHRGPSLRTTTASGKLVTQWCCRSCTMPPLRFLGEDGCWYGMEIRPLSEKEENMVKSQVTA

SEQ ID NO: 5 - protein, 352 aa, Dengue virus
DSGCVINWKGRELKCGSGIFVTNEVHTWTEQYKFQADSPKRLSAAIGKAWEEGVCGIRSATRLENIMWKQISNELNHILLENDMKFTVVVGDANGILTQGKKMIRPQPMEHKYSWKSWGKAKIIGADTQNTTFIIDGPDTPECPDDQRAWNIWEVEDYGFGVFTTNIWLKLRDSYTQMCDHRLMSAAIKDSKAVHADMGYWIESEKNETWKLARASFIEVKTCIWPRSHTLWSNGVLESEMIIPKIYGGPISQHNYRPGYFTQTAGPWHLGKLELDFNLCEGTTVVVDEHCGNRGPSLRTTTVTGKIIHEWCCRSCTLPPLRFRGEDGCWYGMEIRPVKEKEENLVRSMVSA

SEQ ID NO: 6 - protein, 352 aa, Dengue virus
DSGCVINWKGRELKCGSGIFVTNEVHTWTEQYKFQADSPKRLSAAIGKAWEEGVCGIRSATRLENIMWKQISNELNHILLENDMKFTVVVGDVAGILAQGKKMIRPQPMEHKYSWKSWGKAKIIGADVQNTTFIIDGPNTPECPDDQRAWNIWEVEDYGFGIFTTNIWLKLRDSYTQVCDHRLMSAAIKDSKAVHADMGYWIESEKNETWKLARASFIEVKTCIWPKSHTLWSNGVLESEMIIPKIYGGPISQHNYRPGYFTQTAGPWHLGKLELDFDLCEGTTVVVDEHCGNRGPSLRTTTVTGKIIHEWCCRSCTLPPLRFRGEDGCWYGMEIRPVKEKEENLVKSMVSA

SEQ ID NO: 7 - protein, 358 aa, Artificial Sequence
  modified NS1, synthetic seqeunce
DSGCVINWKGRELKCGSGIFVTNEVHTWTEQYKFQADSPKRLSAAIGKAWEEGVCGIRSATRLENIMWKQISNELNHILLENDMKFTVVVGDVSGILAQGKKMIRPQPMEHKYSWKSWGKAKIIGADVQNTTFIIDGPNTPECPDNQRAWNIWEVEDYGFGIFTTNIWLKLRDSYTQVCDHRLMSAAIKDSKAVHADMGYWIESEKNETWKLARASFIEVKTCIWPKSHTLWSNGVLESEMIIPKIYGGPISQHNYRPGYFTQTAGPWHLGKLELDFDLCEGTTVVVDEHCGNRGPSLRTTTVTGKTIHEWCCRSCTLPPLRFKGEDGCWYGMEIRPVKEKEENLVKSMVSAHHHHHH

SEQ ID NO: 8 - protein, 358 aa, Artificial Sequence
  modified NS1, synthetic seqeunce
DSGCVVSWKNKELKCGSGIFITDNVHTWTEQYKFQPESPSKLASAIQKAHEEGICGIRSVTRLENLMWKQITPELNHILSENEVKLTIMTGDIKGIMQAGKRSLRPQPTELKYSWKTWGKAKMLSTESHNQTFLIDGPETAECPNTNRAWNSLEVEDYGFGVFTTNIWLKLKEKQDVFCDSKLMSAAIKDNRAVHADMGYWIESALNDTWKIEKASFIEVKNCHWPKSHTLWSNGVLESEMIIPKNLAGPVSQHNYRPGYHTQITGPWHLGKLEMDFDFCDGTTVVVTEDCGNRGPSLRTTTASGKLITEWCCRSCTLPPLRYRGEDGCWYGMEIRPLKEKEENLVNSLVTAHHHHHH

SEQ ID NO: 9 - protein, 358 aa, Artificial Sequence
  modified NS1, synthetic seqeunce
DMGCVINWKGKELKCGSGIFVTNEVHTWTEQYKFQADSPKRLATAIAGAWENGVCGIRSTTRMENLLWKQIANELNYILWENNIKLTVVVGDTLGVLEQGKRTLTPQPMELKYSWKTWGKAKIVTAETQNSSFIIDGPNTPECPSASRAWNVWEVEDYGFGVFTTNIWLKLREVYTQLCDHRLMSAAVKDERAVHADMGYWIESQKNGSWKLEKASLIEVKTCTWPKSHTLWTNGVLESDMIIPKSLAGPISQHNYRPGYHTQTAGPWHLGKLELDFNYCEGTTVVITESCGTRGPSLRTTTVSGKLIHEWCCRSCTLPPLRYMGEDGCWYGMEIRPISEKEENMVKSLVSAHHHHHH

SEQ ID NO: 10 - protein, 362 aa, Artificial Sequence
  modified NS1, synthetic seqeunce
DMGCVASWSGKELKCGSGIFVVDNVHTWTEQYKFQPESPARLASAILNAHKDGVCGIRSTTRLENVMWKQITNELNYVLWEGGHDLTVVAGDVKGVLTKGKRALTPPVSDLKYSWKTWGKAKIFTPEARNSTFLIDGPDTSECPNERRAWNSLEVEDYGFGMFTTNIWMKFREGSSEVCDHRLMSAAIKDQKAVHADMGYWIESSKNQTWQIEKASLIEVKTCLWPKTHTLWSNGVLESQMLIPKSYAGPFSQHNYRQGYATQTVGPWHLGKLEIDFGECPGTTVTIQEDCDHRGPSLRTTTASGKLVTQWCCRSCTMPPLRFLGEDGCWYGMEIRPLSEKEENMVKSQVTAHHHHHHHHHH

SEQ ID NO: 11 - DNA, 48 bp, Artificial Sequence
  Aptamer, synthetic seqeunce
  misc_feature (19)..(19): derivative of 7-(2thienyl)imidazo[4,5- b]pyridine
  misc_feature (25)..(25): derivative of 7-(2thienyl)imidazo[4,5- b]pyridine
  misc_feature (44)..(44): Biotin-dT
ccccagacggactggtgtnctcggnatggccgtctggggcgcgtagcg

SEQ ID NO: 12 - DNA, 72 bp, Artificial Sequence
  Aptamer, synthetic seqeunce
  misc_feature (11)..(11): derivative of 7-(2thienyl)imidazo[4,5- b]pyridine
  misc_feature (23)..(23): derivative of 7-(2thienyl)imidazo[4,5- b]pyridine
  misc_feature (35)..(35): diol1-derivative of pyrrole2-carbaldehyde
  misc_feature (68)..(68): Biotin-dT
ggctggtccgnctgggaacaagnggcgggagggangggtgtgggtgcgacaagcggaccagcccgcgtagcg

SEQ ID NO: 13 - DNA, 59 bp, Artificial Sequence
  Aptamer, synthetic seqeunce
  misc_feature (15)..(15): derivative of 7-(2thienyl)imidazo[4,5- b]pyridine
  misc_feature (23)..(23): derivative of 7-(2thienyl)imidazo[4,5- b]pyridine
  misc_feature (55)..(55): Biotin-dT
ccgcttgtcatctancctggccntgtggtactgtaacggctgacaagcggcgcgtagcg

SEQ ID NO: 14 - DNA, 57 bp, Artificial Sequence
  Aptamer, synthetic seqeunce
  misc_feature (18)..(18): derivative of 7-(2thienyl)imidazo[4,5- b]pyridine
  misc_feature (28)..(28): derivative of 7-(2thienyl)imidazo[4,5- b]pyridine
  misc_feature (53)..(53): Biotin-dT
cggcggagacgtaacgcntatcaaatcnaaacagcttagggtccgccgcgcgtagcg

SEQ ID NO: 15 - DNA, 97 bp, Artificial Sequence
  Aptamer, synthetic seqeunce
  misc_feature (1)..(1): Biotin-dT
  misc_feature (49)..(49): derivative of 7-(2thienyl)imidazo[4,5- b]pyridine
  misc_feature (61)..(61): derivative of 7-(2thienyl)imidazo[4,5- b]pyridine
ttttcgcactccatgatatggtctactgagcgagacgatgctgctaaantacgccgtggtnacgaagacagacaagcggagtagttagaccgtgaaa

SEQ ID NO: 16 - DNA, 88 bp, Artificial Sequence
  Aptamer, synthetic seqeunce
  misc_feature (44)..(44): derivative of 7-(2thienyl)imidazo[4,5- b]pyridine
  misc_feature (56)..(56): derivative of 7-(2thienyl)imidazo[4,5- b]pyridine
  misc_feature (84)..(84): Biotin-dT
gcactccatgatatggtctactgagcgagacgatgctgctaaantacgccgtggtnacgaagacagacaagcggagtgtcgcgtagcg

SEQ ID NO: 17 - DNA, 78 bp, Artificial Sequence
  Aptamer, synthetic seqeunce
  misc_feature (1)..(1): Biotin-dT
  misc_feature (21)..(21): derivative of 7-(2thienyl)imidazo[4,5- b]pyridine
  misc_feature (42)..(42): derivative of 7-(2thienyl)imidazo[4,5- b]pyridine
  misc_feature (48)..(48): derivative of 7-(2thienyl)imidazo[4,5- b]pyridine
tgatatggtctactgtgtgangtcctacaatggactggtgtnctcggnatggccattgacaagcggagtagttagacc

SEQ ID NO: 18 - DNA, 42 bp, Artificial Sequence
  Aptamer, synthetic seqeunce
  misc_feature (16)..(16): derivative of 7-(2thienyl)imidazo[4,5- b]pyridine
  misc_feature (22)..(22): derivative of 7-(2thienyl)imidazo[4,5- b]pyridine
  misc_feature (38)..(38): Biotin-dT
cagacggactggtgtnctcggnatggccgtctgcgcgtagcg

SEQ ID NO: 19 - DNA, 97 bp, Artificial Sequence
  Aptamer, synthetic seqeunce
  misc_feature (1)..(1): Biotin-dT
  misc_feature (34)..(34): derivative of 7-(2thienyl)imidazo[4,5- b]pyridine
  misc_feature (46)..(46): derivative of 7-(2thienyl)imidazo[4,5- b]pyridine
  misc_feature (58)..(58): diol1-derivative of pyrrole2-carbaldehyde
ttttcgcactccatgatatggtctactggtccgnctgggaacaagnggcgggagggangggtgtgggtgcgacaagcggagtagttagaccgtcaaa

SEQ ID NO: 20 - DNA, 97 bp, Artificial Sequence; identical to SEQ ID NO: 19
  Aptamer, synthetic seqeunce
  misc_feature (1)..(1): Biotin-dT
  misc_feature (34)..(34): derivative of 7-(2thienyl)imidazo[4,5- b]pyridine
  misc_feature (46)..(46): derivative of 7-(2thienyl)imidazo[4,5- b]pyridine
  misc_feature (58)..(58): n = Diol1-dPx
  misc_feature (58)..(58): diol1-derivative of pyrrole2-carbaldehyde

SEQ ID NO: 21 - DNA, 84 bp, Artificial Sequence
  Aptamer, synthetic seqeunce
  misc_feature (1)..(1): Biotin-dT
  misc_feature (34)..(34): derivative of 7-(2thienyl)imidazo[4,5- b]pyridine
  misc_feature (46)..(46): derivative of 7-(2thienyl)imidazo[4,5- b]pyridine
  misc_feature (58)..(58): diol1-derivative of pyrrole2-carbaldehyde
ttttcgcactccatgatatggtctactggtccgnctgggaacaagnggcgggagggangggtgtgggtgcgacaagcggagtag

SEQ ID NO: 22 - DNA, 74 bp, Artificial Sequence
  Aptamer, synthetic seqeunce
  misc_feature (1)..(1): Biotin-dT
  misc_feature (24)..(24): derivative of 7-(2thienyl)imidazo[4,5- b]pyridine
  misc_feature (36)..(36): derivative of 7-(2thienyl)imidazo[4,5- b]pyridine
  misc_feature (48)..(48): diol1-derivative of pyrrole2-carbaldehyde
ncatgatatggtctactggtccgnctgggaacaagnggcgggagggangggtgtgggtgcgacaagcggagtag

SEQ ID NO: 23 - DNA, 87 bp, Artificial Sequence
  Aptamer, synthetic seqeunce
  misc_feature (18)..(18): derivative of 7-(2thienyl)imidazo[4,5- b]pyridine
  misc_feature (30)..(30): derivative of 7-(2thienyl)imidazo[4,5- b]pyridine
  misc_feature (42)..(42): diol1-derivative of pyrrole2-carbaldehyde
  misc_feature (83)..(83): Biotin-dT
gacggtctactggtccgnctgggaacaagnggcgggagggangggtgtgggtgcgacaagcggagtagttagaccgtccgcgtagcg

SEQ ID NO: 24 - DNA, 77 bp, Artificial Sequence
  Aptamer, synthetic seqeunce
  misc_feature (15)..(15): derivative of 7-(2thienyl)imidazo[4,5- b]pyridine
  misc_feature (27)..(27): derivative of 7-(2thienyl)imidazo[4,5- b]pyridine
  misc_feature (39)..(39): diol1-derivative of pyrrole2-carbaldehyde
  misc_feature (73)..(73): Biotin-dT
ggtctactggtccgnctgggaacaagnggcgggagggangggtgtgggtgcgacaagcggagtagacccgcgtagcg

SEQ ID NO: 25 - DNA, 61 bp, Artificial Sequence
  Aptamer, synthetic seqeunce
  misc_feature (7)..(7): derivative of 7-(2thienyl)imidazo[4,5- b]pyridine
  misc_feature (19)..(19): derivative of 7-(2thienyl)imidazo[4,5- b]pyridine
  misc_feature (31)..(31): diol1-derivative of pyrrole2-carbaldehyde
  misc_feature (57)..(57): Biotin-dT
ggtccgnctgggaacaagnggcgggagggangggtgtgggtgcgacaagcggcgcgtagcg

SEQ ID NO: 26 - DNA, 78 bp, Artificial Sequence
  Aptamer, synthetic seqeunce
  misc_feature (1)..(1): Biotin-dT
  misc_feature (31)..(31): derivative of 7-(2thienyl)imidazo[4,5- b]pyridine
  misc_feature (39)..(39): derivative of 7-(2thienyl)imidazo[4,5- b]pyridine
tgatatggtctactgaagtgttgtcatctancctggccntgtggtactgtaacggctgacaagcggagtagttagacc

SEQ ID NO: 27 - DNA, 78 bp, Artificial Sequence
  Aptamer, synthetic seqeunce
  misc_feature (1)..(1): Biotin-dT
  misc_feature (31)..(31): derivative of 7-(2thienyl)imidazo[4,5- b]pyridine
  misc_feature (36)..(36): derivative of 7-(2thienyl)imidazo[4,5- b]pyridine
  misc_feature (46)..(46): derivative of 7-(2thienyl)imidazo[4,5- b]pyridine
tgatatggtctactgtggcgcgagggaatcnacgcntatcaaatanaaacagctaatgacaagcggagtagttagacc

SEQ ID NO: 28 - DNA, 78 bp, Artificial Sequence
  Aptamer, synthetic seqeunce
  misc_feature (1)..(1): Biotin-dT
  misc_feature (41)..(41): derivative of 7-(2thienyl)imidazo[4,5- b]pyridine
  misc_feature (52)..(52): derivative of 7-(2thienyl)imidazo[4,5- b]pyridine
tgatatggtctactgaggagcgcatgtcgagataccaaccnccatccaatcnttcttgacaagcggagtagttagacc

SEQ ID NO: 29 - DNA, 78 bp, Artificial Sequence
  Aptamer, synthetic seqeunce
  misc_feature (1)..(1): Biotin-dT
  misc_feature (32)..(32): derivative of 7-(2thienyl)imidazo[4,5- b]pyridine
  misc_feature (45)..(45): derivative of 7-(2thienyl)imidazo[4,5- b]pyridine
ttgatatggtctactgacgccggggcccgtantcagacgtatacncatcagggcacatacaagcggagtagttagacc

SEQ ID NO: 30 - DNA, 47 bp, Artificial Sequence
  Aptamer, synthetic seqeunce
  misc_feature (12)..(12): derivative of 7-(2thienyl)imidazo[4,5- b]pyridine
  misc_feature (25)..(25): derivative of 7-(2thienyl)imidazo[4,5- b]pyridine
  misc_feature (43)..(43): Biotin-dT
cgaggcccgtantcagacgtatacncatcagggcctcgcgcgtagcg

SEQ ID NO: 31 - DNA, 61 bp, Artificial Sequence
  Aptamer, synthetic seqeunce
  misc_feature (17)..(17): derivative of 7-(2thienyl)imidazo[4,5- b]pyridine
  misc_feature (57)..(57): Biotin-dT
ggcagcgcgtcgattgnccaatcttagccaacccaaaattacaagcgctgcccgcgtagcg

SEQ ID NO: 32 - DNA, 47 bp, Artificial Sequence
  Aptamer, synthetic seqeunce
  misc_feature (8)..(8): derivative of 7-(2thienyl)imidazo[4,5- b]pyridine
  misc_feature (28)..(28): derivative of 7-(2thienyl)imidazo[4,5- b]pyridine
  misc_feature (43)..(43): Biotin-dT
gctgcctngtaccaaccccctccaatcnattaggcagccgcgtagcg

SEQ ID NO: 33 - DNA, 51 bp, Artificial Sequence
  Aptamer, synthetic seqeunce
  misc_feature (11)..(11): derivative of 7-(2thienyl)imidazo[4,5- b]pyridine
  misc_feature (30)..(30): derivative of 7-(2thienyl)imidazo[4,5- b]pyridine
  misc_feature (47)..(47): Biotin-dT
cgtgcgacgangtccaaccagtcccaatcnacaagtcgcacgcgcgtagcg

SEQ ID NO: 34 - DNA, 59 bp, Artificial Sequence
  Aptamer, synthetic seqeunce
  misc_feature (12)..(12): derivative of 7-(2thienyl)imidazo[4,5- b]pyridine
  misc_feature (27)..(27): diol1-derivative of pyrrole2-carbaldehyde
  misc_feature (55)..(55): Biotin-dT
gcggtccgtgcngtcgccaatccgtgntccaaccccgacaagcggaccgccgcgtagcg

SEQ ID NO: 35 - DNA, 52 bp, Artificial Sequence
  Aptamer, synthetic seqeunce
  misc_feature (12)..(12): derivative of 7-(2thienyl)imidazo[4,5- b]pyridine
  misc_feature (23)..(23): diol1-derivative of pyrrole2-carbaldehyde
  misc_feature (48)..(48): Biotin-dT
gcccgctttcgnccaacccgtgntccaatcccagaaagcgggccgcgtagcg

SEQ ID NO: 36 - DNA, 56 bp, Artificial Sequence
  Aptamer, synthetic seqeunce
  misc_feature (13)..(13): derivative of 7-(2thienyl)imidazo[4,5- b]pyridine
  misc_feature (26)..(26): diol1-derivative of pyrrole2-carbaldehyde
  misc_feature (52)..(52): Biotin-dT
cgcccgtcaaggnctccaatccgtgntccaaccagttttgacgggcgcgcgtagcg

SEQ ID NO: 37 - DNA, 46 bp, Artificial Sequence
  Aptamer, synthetic seqeunce
  misc_feature (28)..(28): derivative of 7-(2thienyl)imidazo[4,5- b]pyridine
  misc_feature (42)..(42): Biotin-dT
gcccgcgtgctcaaccttaccaatctgncacgcgggccgcgtagcg

SEQ ID NO: 38 - DNA, 48 bp, Artificial Sequence
  Aptamer, synthetic seqeunce
  misc_feature (9)..(9): derivative of 7-(2thienyl)imidazo[4,5- b]pyridine
  misc_feature (29)..(29): derivative of 7-(2thienyl)imidazo[4,5- b]pyridine
  misc_feature (44)..(44): Biotin-dT
gccctgcgngctcaaccttaccaatctgncacgcagggccgcgtagcg

SEQ ID NO: 39 - DNA, 85 bp, Artificial Sequence
  Aptamer, synthetic seqeunce
  misc_feature (1)..(1): Biotin-dT
  misc_feature (33)..(33): derivative of 7-(2thienyl)imidazo[4,5- b]pyridine
  misc_feature (46)..(46): derivative of 7-(2thienyl)imidazo[4,5- b]pyridine
tactccatgatatggtctactgatagtactccngtttaactctganacttgacgtccattcatagacaagcggagtagttagacc

SEQ ID NO: 40 - DNA, 78 bp, Artificial Sequence
  Aptamer, synthetic seqeunce
  misc_feature (1)..(1): Biotin-dT
  misc_feature (32)..(32): derivative of 7-(2thienyl)imidazo[4,5- b]pyridine
tgatatggtctactggggcttggtcttgcgtntgcagattaacttgcgtgccagtaagacaagcggagtagttagacc

SEQ ID NO: 41 - DNA, 78 bp, Artificial Sequence
  Aptamer, synthetic seqeunce
  misc_feature (1)..(1): Biotin-dT
  misc_feature (36)..(36): derivative of 7-(2thienyl)imidazo[4,5- b]pyridine
  misc_feature (46)..(46): derivative of 7-(2thienyl)imidazo[4,5- b]pyridine
tgatatggtctactgtctcaacggttgtcaaacggntatcacggcnacacacctgcggacaagcggagtagttagacc

SEQ ID NO: 42 - DNA, 57 bp, Artificial Sequence
  Aptamer, synthetic seqeunce
  misc_feature (17)..(17): derivative of 7-(2thienyl)imidazo[4,5- b]pyridine
  misc_feature (27)..(27): derivative of 7-(2thienyl)imidazo[4,5- b]pyridine
  misc_feature (53)..(53): Biotin-dT
ctccgctgtcaaacggntatcacggcnacacacctgcggacagcggagcgcgtagcg

SEQ ID NO: 43 - DNA, 78 bp, Artificial Sequence
  Aptamer, synthetic seqeunce
  misc_feature (1)..(1): Biotin-dT
  misc_feature (21)..(21): derivative of 7-(2thienyl)imidazo[4,5- b]pyridine
  misc_feature (34)..(34): derivative of 7-(2thienyl)imidazo[4,5- b]pyridine
tgatatggtctactgtcacanatcgccgtaaagncgaagagctgcggaatctaaggtgacaagcggagtagttagacc

SEQ ID NO: 44 - DNA, 78 bp, Artificial Sequence
  Aptamer
  misc_feature (1)..(1): Biotin-dT
  misc_feature (26)..(26): derivative of 7-(2thienyl)imidazo[4,5- b]pyridine
  misc_feature (39)..(39): derivative of 7-(2thienyl)imidazo[4,5- b]pyridine
tgatatggtctactgtataatccgcnttcgtcatgtggnttggatctgggtctggcagacaagcggagtagttagacc

SEQ ID NO: 45 - DNA, 78 bp, Artificial Sequence
  Aptamer, synthetic seqeunce
  misc_feature (1)..(1): Biotin-dT
  misc_feature (21)..(21): derivative of 7-(2thienyl)imidazo[4,5- b]pyridine
  misc_feature (36)..(36): derivative of 7-(2thienyl)imidazo[4,5- b]pyridine
  misc_feature (41)..(41): derivative of 7-(2thienyl)imidazo[4,5- b]pyridine
tgatatggtctactgcccaancttgtctgtaagggnttggntagggctggcaaaaaagacaagcggagtagttagacc

SEQ ID NO: 46 - DNA, 74 bp, Artificial Sequence
  Aptamer, synthetic seqeunce
  misc_feature (18)..(18): derivative of 7-(2thienyl)imidazo[4,5- b]pyridine
  misc_feature (30)..(30): derivative of 7-(2thienyl)imidazo[4,5- b]pyridine
  misc_feature (70)..(70): Biotin-dT
cggccgatgctgctaaantacgccgtggtnacgaagacagacaagcggagtagttagaccggccgcgcgtagcg

SEQ ID NO: 47 - DNA, 59 bp, Artificial Sequence
  Aptamer, synthetic seqeunce
  misc_feature (9)..(9): derivative of 7-(2thienyl)imidazo[4,5- b]pyridine
  misc_feature (21)..(21): derivative of 7-(2thienyl)imidazo[4,5- b]pyridine
  misc_feature (55)..(55): Biotin-dT
gcgccaaantacgccgtggtncgaagacagacaagcggagtagttggcgccgcgtagcg

SEQ ID NO: 48 - DNA, 72 bp, Artificial Sequence
  Aptamer, synthetic seqeunce
  misc_feature (36)..(36): derivative of 7-(2thienyl)imidazo[4,5- b]pyridine
  misc_feature (48)..(48): derivative of 7-(2thienyl)imidazo[4,5- b]pyridine
  misc_feature (68)..(68): Biotin-dT
gcactccgtctactgagcgagacgatgctgctaaantacgccgtggtnacgaagacggagtgtcgcgtagcg

SEQ ID NO: 49 - DNA, 74 bp, Artificial Sequence
  aptamer, synthetic seqeunce
  misc_feature (35)..(35): derivative of 7-(2thienyl)imidazo[4,5- b]pyridine
  misc_feature (47)..(47): derivative of 7-(2thienyl)imidazo[4,5- b]pyridine
  misc_feature (70)..(70): Biotin-dT
gcactccgctactgagcgagacgatgctgctaaantacgccgtggtnacgaagacagcggagtgtcgcgtagcg

SEQ ID NO: 50 - DNA, 72 bp, Artificial Sequence
  Aptamer, synthetic seqeunce
  misc_feature (23)..(23): derivative of 7-(2thienyl)imidazo[4,5- b]pyridine
  misc_feature (35)..(35): diol1-derivative of pyrrole2-carbaldehyde
  misc_feature (68)..(68): Biotin-dT
ggctggtccgactgggaacaagnggcgggagggangggtgtgggtgcgacaagcggaccagcccgcgtagcg

SEQ ID NO: 51 - DNA, 19 bp, Artificial Sequence
  Core sequences, synthetic seqeunce
  misc_feature (9)..(9): derivative of 7-(2thienyl)imidazo[4,5- b]pyridine
  misc_feature (15)..(15): derivative of 7-(2thienyl)imidazo[4,5- b]pyridine
actggtgtnctcggnatgg

SEQ ID NO: 52 - DNA, 41 bp, Artificial Sequence
  Core sequences, synthetic seqeunce
  misc_feature (11)..(11): derivative of 7-(2thienyl)imidazo[4,5- b]pyridine
  misc_feature (23)..(23): diol1-derivative of pyrrole2-carbaldehyde or diol1-derivative of 2-nitro-4-propynylpyrrole
tgggaacaagnggcgggagggangggtgtgggtgcgacaag

SEQ ID NO: 53 - DNA, 30 bp, Artificial Sequence
  Core sequences, synthetic seqeunce
  misc_feature (5)..(5): derivative of 7-(2thienyl)imidazo[4,5- b]pyridine
  misc_feature (13)..(13): derivative of 7-(2thienyl)imidazo[4,5- b]pyridine
tctancctggccntgtggtactgtaacggc

SEQ ID NO: 54 - DNA, 29 bp, Artificial Sequence
  Core sequences, synthetic seqeunce
  misc_feature (11)..(11): derivative of 7-(2thienyl)imidazo[4,5- b]pyridine
  misc_feature (21)..(21): derivative of 7-(2thienyl)imidazo[4,5- b]pyridine
gacgtaacgcntatcaaatcnaaacagct

SEQ ID NO: 55 - DNA, 65 bp, Artificial Sequence
  Core sequences, synthetic seqeunce
  misc_feature (37)..(37): derivative of 7-(2thienyl)imidazo[4,5- b]pyridine
  misc_feature (49)..(49): derivative of 7-(2thienyl)imidazo[4,5- b]pyridine
atgatatggtctactgagcgagacgatgctgctaaantacgccgtggtnacgaagacagacaagc

SEQ ID NO: 56 - DNA, 33 bp, Artificial Sequence
  Core sequences, synthetic seqeunce
  misc_feature (10)..(10): derivative of 7-(2thienyl)imidazo[4,5- b]pyridine
  misc_feature (15)..(15): derivative of 7-(2thienyl)imidazo[4,5- b]pyridine
  misc_feature (25)..(25): derivative of 7-(2thienyl)imidazo[4,5- b]pyridine
gagggaatcnacgcntatcaaatanaaacagct

SEQ ID NO: 57 - DNA, 28 bp, Artificial Sequence
  Core sequences, synthetic seqeunce
  misc_feature (7)..(7): derivative of 7-(2thienyl)imidazo[4,5- b]pyridine
  misc_feature (17)..(17): derivative of 7-(2thienyl)imidazo[4,5- b]pyridine
aaacggntatcacggcnacacacctgcg

(19) United States
(12) Patent Application Publication
(10) Pub. No.: US 2022/0315926 A1
(43) Pub. Date: Oct. 6, 2022

(54) An Aptamer for Dengue Virus and Related Methods and Products

(71) Applicants: Agency for Science, Technology and Research, Singapore (SG); Tan Tock Seng Hospital PTE Ltd, Singapore (SG)

(72) Inventors: Ichiro Hirao, Singapore (SG); Ken-ichiro Matsunaga, Singapore (SG); Michiko Hirao, Singapore (SG); William Sun, Singapore (SG); Yee Sin Leo, Singapore (SG)

(21) Appl. No.: 17/596,847

(22) PCT Filed: Jun. 18, 2020
(86) PCT No.: PCT/SG2020/050342

§ 371 Date: Dec. 20, 2021

(30) Foreign Application Priority Data
Jun. 21, 2019 (SG) 10201905754 W

(51) Int. Cl. C12N 15/115; G01N 33/569

(52) U.S. Cl. CPC C12N 15/115; G01N 33/56983; C12N 2310/16; C12N 2320/34; C12N 2310/33

(57) ABSTRACT

There is provided an aptamer for dengue virus, optionally an aptamer for dengue virus NS1 protein. The aptamer comprising at least one unnatural base, wherein the unnatural base may be 7-(2thienyl)imidazo[4,5-b]pyridine (Ds), pyrrole2-carbaldehyde (Pa) or 2-nitro-4-propynylpyrrole (Px). The aptamers of the invention may be used to identify a dengue infection in a subject. Also provided are mixtures and kits comprising the aptamer.

### TECHNICAL FIELD

The present disclosure relates broadly to an aptamer for dengue virus and related methods, mixtures, kits and nucleic acid molecules.

### BACKGROUND

Dengue is a widespread mosquito-borne viral disease, with four categorized serotypes. Although there is no drug therapy available, early detection and medical care reduce morbidity and mortality. A secondary infection with a different serotype results in more severe disease. A commercially available dengue vaccine has been developed, but not for use in previously uninfected people.

Dengue is an arthropod-borne flavivirus with four main serological types (DEN1-4). Worldwide dengue virus (DENV) infections have been estimated at more than 390 million annually, and the recent dramatic and global increase in incidence suggests that approximately 40% of the world's population is now at risk. The symptoms range from mild in most cases to severe and occasionally fatal. Infection with one DENV serotype provides long-term protection from re-infection with the same serotype, but may enhance disease from a secondary heterotypic infection. A secondary DENV infection is the greatest risk factor for severe disease such as Dengue Hemorrhagic Fever and Dengue Shock Syndrome. Antibody-dependent enhancement (ADE) is thought to be one of the mechanisms responsible for severe dengue. The immune history of a person is important for understanding subsequent disease risk, pathogenesis, and protection, and thus the ability to elucidate previous infected serotype is invaluable to study. Diagnostic methods for serotype identification in previously infected patients are helpful to elucidate whether the sequence of dengue serotype infection affects the severity of the disease.

There is currently no specific treatment for dengue, but early diagnostics prompt proper medical attention and reduce the risk of fatality. Dengvaxia, a dengue vaccine, was developed by Sanofi Pasteur. However, analyses revealed that the vaccination of persons who have never been infected with dengue led to a higher risk of more severe symptoms when they became infected after the vaccination. Consequently, the World Health Organization (WHO) advised the use of the vaccine only in people previously infected with dengue. The effectiveness of the vaccine could be different for people previously infected with different serotypes of dengue, and more research is needed to understand the mechanisms. Therefore, the development of detection methods for not only current infection but also past infection, including the serotype identification, is an urgent worldwide task.

In the early phase of DENV infection (e.g. within one week after fever onset) the viral RNA can be identified by RT-qPCR. Virus-related materials, such as the envelope protein and nonstructural protein 1 (NS1), can also be detected by an enzyme-linked immunosorbent assay (ELISA) or lateral flow assay (LFA), using antibodies to the antigens. RT-qPCR is useful for serotype identification in the early phase, but not in the later phase. For the serotype-specific NS1 detection in the early phase, the generation of antibodies to each NS1 serotype has been described, but these antibodies are not commercially available. In the later phase, the patients' IgM and IgG antibodies to viral-related antigens, such as viral particles and NS1 proteins, are detectable by ELISA or LFA. However, the detection reliabilities are still limited, and the serotype identification remains difficult.

DNA aptamers are single-stranded DNA fragments that bind specifically to target molecules, and are considered to be antibody alternatives. DNA aptamers are initially obtained by an evolutionary engineering method called SELEX (Systematic Evolution of Ligands by EXponential enrichment), involving repetitive cycles of selection and PCR amplification using DNA libraries. Once the appropriate aptamer sequences are determined, they can be chemically synthesized on a large GMP scale. Although many DNA aptamers have been reported, their applications remain limited due to the insufficient affinities to their targets (K_{D} values 10⁻⁷-10⁻⁹ M).

Thus, there is a need to provide an alternative aptamer for dengue virus and related methods and kits.

### SUMMARY

In one aspect, there is provided an aptamer for dengue virus (DENV), the aptamer comprising at least one unnatural base.

In one embodiment, the at least one unnatural base resides in a loop structure and/or a bulge of the aptamer.

In one embodiment, the at least one unnatural base is selected from the group consisting of: 7-(2thienyl)imidazo[4,5-b]pyridine (Ds); 7-(2,2′-bithien-5-yl)imidazo[4,5-b]pyridin-3-yl group (Dss); pyrrole2-carbaldehyde (Pa); 2-nitro-4-propynylpyrrole (Px); 7-(2,2′,5′,2″-terthien-5-yl)imidazo[4,5-b]pyridin-3-yl group (Dsss); 2-amino-6-(2-thienyl)purin-9-yl group (5); 2-amino-6-(2,2′-bithien-5-yl)purin-9-yl group (ss); 2-amino-6-(2,2′,5′,2″-terthien-5-yl)purin-9-yl group (sss); 4-(2-thienyl)-pyrrolo[2,3-b]pyridin-1-yl group (dDsa); 4-(2,2′-bithien-5-yl)-pyrrolo[2,3-b]pyridin-1-yl group (Dsas); 4-[2-(2-thiazolyl)thien-5-yl]pyrrolo[2,3-b]pyridin-1-yl group (Dsav); 4-(2-thiazolyI)-pyrrolo[2,3-b]pyridin-1-yl group (dDva); 4-[5-(2-thienyl)thiazol-2-yl]pyrrolo[2,3-b]pyridin-1-yl group (Dvas); 4-(2-imidazolyl)-pyrrolo[2,3-b]pyridin-1-yl group (dDia); derivatives thereof; and combinations thereof.

In one embodiment, the aptamer comprises a DNA-based aptamer.

In one embodiment, the dissociation constant of the aptamer for DENV is no more than 200 pM.

In one embodiment, the aptamer is capable of binding to the NS1 protein of DENV.

In one embodiment, the aptamer is capable of binding specifically to a single serotype of DENV selected from the group consisting of serotype 1, serotype 2, serotype 3 and serotype 4.

In one embodiment, the aptamer comprises a sequence set out in the table below:

| SEQ | Sequence (L = Biotin-dT, x = dDs, d = Diol1-dPa, |
|---|---|
| ID NO. | y = DioL1-dPx, w = Diol1-dPa or Diol1-dPx) |
| 11 | CCCCAGACGGACTGGTGTxCTCGGxATGGCCGTCTGGGGCGCGL |
| | AGCG |
| 12 | GGCTGGTCCGxCTGGGAACAAGxGGCGGGAGGGAdGGGTGTGG |
| | GTGCGACAAGCGGACCAGCCCGCGLAGCG |
| 13 | CCGCTTGTCATCTAxCCTGGCCxTGTGGTACTGTAACGGCTGACA |
| | AGCGGCGCGLAGCG |
| 14 | CGGCGGAGACGTAACGCxTATCAAATCxAAACAGCTTAGGGTCC |
| | GCCGCGCGLAGCG |
| 15 | Biotin-TTTCGCACTCCATGATATGGTCTACTGAGCGAGACGATGCT |
| | GCTAAAxTACGCCGTGGTxACGAAGACAGACAAGCGGAGTAGTTA |
| | GACCGTGAAA |
| 16 | GCACTCCATGATATGGTCTACTGAGCGAGACGATGCTGCTAAAxT |
| | ACGCCGTGGTxACGAAGACAGACAAGCGGAGTGTCGCGLAGCG |
| 17 | LGATATGGTCTACTGTGTGAxGTCCTACAATGGACTGGTGTxCTC |
| | GGxATGGCCATTGACAAGCGGAGTAGTTAGACC |
| 18 | CAGACGGACTGGTGTxCTCGGxATGGCCGTCTGCGCGLAGCG |
| 19 | LTTTCGCACTCCATGATATGGTCTACTGGTCCGxCTGGGAACAAG |
| | xGGCGGGAGGGAdGGGTGTGGGTGCGACAAGCGGAGTAGTTAG |
| | ACCGTCAAA |
| 20 | Biotin-TTTCGCACTCCATGATATGGTCTACTGGTCCGxCTGGGAAC |
| | AAGxGGCGGGAGGGAyGGGTGTGGGTGCGACAAGCGGAGTAGT |
| | TAGACCGTCAAA |
| 21 | LTTTCGCACTCCATGATATGGTCTACTGGTCCGxCTGGGAACAAG |
| | xGGCGGGAGGGAdGGGTGTGGGTGCGACAAGCGGAGTAG |
| 22 | LCATGATATGGTCTACTGGTCCGxCTGGGAACAAGxGGCGGGAG |
| | GGAdGGGTGTGGGTGCGACAAGCGGAGTAG |
| 23 | GACGGTCTACTGGTCCGxCTGGGAACAAGxGGCGGGAGGGAdG |
| | GGTGTGGGTGCGACAAGCGGAGTAGTTAGACCGTCCGCGLAGC |
| | G |
| 24 | GGTCTACTGGTCCGxCTGGGAACAAGxGGCGGGAGGGAdGGGT |
| | GTGGGTGCGACAAGCGGAGTAGACCCGCGLAGCG |
| 25 | GGTCCGxCTGGGAACAAGxGGCGGGAGGGAdGGGTGTGGGTGC |
| | GACAAGCGGCGCGLAGCG |
| 26 | LGATATGGTCTACTGAAGTGTTGTCATCTAxCCTGGCCxTGTGGTA |
| | CTGTAACGGCTGACAAGCGGAGTAGTTAGACC |
| 27 | LGATATGGTCTACTGTGGCGCGAGGGAATCxACGCxTATCAAATA |
| | xAAACAGCTAATGACAAGCGGAGTAGTTAGACC |
| 28 | LGATATGGTCTACTGAGGAGCGCATGTCGAGATACCAACCxCCAT |
| | CCAATCxTTCTTGACAAGCGGAGTAGTTAGACC |
| 29 | LTGATATGGTCTACTGACGCCGGGGCCCGTAxTCAGACGTATACx |
| | CATCAGGGCACATACAAGCGGAGTAGTTAGACC |
| 30 | CGAGGCCCGTAxTCAGACGTATACxCATCAGGGCCTCGCGCGLA |
| | GCG |
| 31 | GGCAGCGCGTCGATTGxCCAATCTTAGCCAACCCAAAATTACAAG |
| | CGCTGCCCGCGLAGCG |
| 32 | GCTGCCTxGTACCAACCCCCTCCAATCxATTAGGCAGCCGCGLAG |
| | CG |
| 33 | CGTGCGACGAxGTCCAACCAGTCCCAATCxACAAGTCGCACGCG |
| | CGLAGCG |
| 34 | GCGGTCCGTGCxGTCGCCAATCCGTGdTCCAACCCCGACAAGCG |
| | GACCGCCGCGLAGCG |
| 35 | GCCCGCTTTCGxCCAACCCGTGdTCCAATCCCAGAAAGCGGGCC |
| | GCGLAGCG |
| 36 | CGCCCGTCAAGGxCTCCAATCCGTGdTCCAACCAGTTTTGACGG |
| | GCGCGCGLAGCG |
| 37 | GCCCGCGTGCTCAACCTTACCAATCTGxCACGCGGGCCGCGLAG |
| | CG |
| 38 | GCCCTGCGxGCTCAACCTTACCAATCTGxCACGCAGGGCCGCGL |
| | AGCG |
| 39 | LACTCCATGATATGGTCTACTGATAGTACTCCxGTTTAACTCTGAx |
| | ACTTGACGTCCATTCATAGACAAGCGGAGTAGTTAGACC |
| 40 | LGATATGGTCTACTGGGGCTTGGTCTTGCGTxTGCAGATTAACTT |
| | GCGTGCCAGTAAGACAAGCGGAGTAGTTAGACC |
| 41 | LGATATGGTCTACTGTCTCAACGGTTGTCAAACGGxTATCACGGC |
| | xACACACCTGCGGACAAGCGGAGTAGTTAGACC |
| 42 | CTCCGCTGTCAAACGGxTATCACGGCxACACACCTGCGGACAGC |
| | GGAGCGCGLAGCG |
| 43 | LGATATGGTCTACTGTCACAxATCGCCGTAAAGxCGAAGAGCTGC |
| | GGAATCTAAGGTGACAAGCGGAGTAGTTAGACC |
| 44 | LGATATGGTCTACTGTATAATCCGCxTTCGTCATGTGGxTTGGATC |
| | TGGGTCTGGCAGACAAGCGGAGTAGTTAGACC |
| 45 | LGATATGGTCTACTGCCCAAxCTTGTCTGTAAGGGxTTGGxTAGG |
| | GCTGGCAAAAAAGACAAGCGGAGTAGTTAGACC |
| 46 | CGGCCGATGCTGCTAAAxTACGCCGTGGTxACGAAGACAGACAA |
| | GCGGAGTAGTTAGACCGGCCGCGCGLAGCG |
| 47 | GCGCCAAAxTACGCCGTGGTxCGAAGACAGACAAGCGGAGTAGT |
| | TGGCGCCGCGLAGCG |
| 48 | GCACTCCGTCTACTGAGCGAGACGATGCTGCTAAAxTACGCCGT |
| | GGTxACGAAGACGGAGTGTCGCGLAGCG |
| 49 | GCACTCCGCTACTGAGCGAGACGATGCTGCTAAAxTACGCCGTG |
| | GTxACGAAGACAGCGGAGTGTCGCGLAGCG |
| 50 | GGCTGGTCCGACTGGGAACAAGxGGCGGGAGGGAdGGGTGTGG |
| | GTGCGACAAGCGGACCAGCCCGCGLAGCG |
| 51 | ACTGGTGTxCTCGGxATGG |
| 52 | TGGGAACAAGxGGCGGGAGGGAwGGGTGTGGGTGCGACAAG |
| 53 | TCTAxCCTGGCCxTGTGGTACTGTAACGGC |
| 54 | GACGTAACGCxTATCAAATCxAAACAGCT |
| 55 | ATGATATGGTCTACTGAGCGAGACGATGCTGCTAAAxTACGCCGT |
| | GGTxACGAAGACAGACAAGC |
| 56 | GAGGGAATCxACGCxTATCAAATAxAAACAGCT |
| 57 | AAACGGxTATCACGGCxACACACCTGCG |

or; a sequence sharing at least 75% sequence identity thereto; or a sequence differing by one, two, three, four, five, six, seven, eight, nine or ten bases thereto; or portions thereof.

In one aspect, there is provided a mixture of aptamers that are specific to different serotypes, the mixture comprising at least two, at least three or at least four of the aptamers.

In one aspect, there is provided a method of identifying a DENV infection in a subject, the method comprising: contacting a sample of the subject with the aptamer or the mixture of aptamers; and detecting a binding event at the aptamer(s).

In one embodiment, the method is a method of identifying a current DENV infection in the subject, and a binding event at any of the aptamer(s) is indicative of a current DENV infection in the subject, optionally wherein the bound aptamer is specific to single DENV serotype and the binding event is indicative of a current DENV infection of said serotype in the subject.

In one embodiment, wherein where the subject is indicated for a current DENV infection, the method comprises further contacting a sample of the subject with the aptamer or the mixture of aptamers in the presence of a DENV protein; and detecting a binding event at the aptamer(s), wherein an absence of a binding event at any of the aptamer(s) is indicative that the current DENV infection is a secondary or further DENV infection, optionally wherein the unbound aptamer(s) is specific to a DENV serotype and the absence of the binding event(s) is indicative of a past DENV infection of said serotype(s) in the subject.

In one embodiment, wherein the method is a method of identifying a past DENV infection in the subject, the contacting step is performed in the presence of a DENV protein, and an absence of a binding event at any of the aptamer(s) is indicative of a past DENV infection in the subject, optionally wherein the unbound aptamer(s) is specific to a DENV serotype and the absence of the binding event(s) is indicative of a past DENV infection of said serotype(s) in the subject.

In one embodiment, the method comprises a competitive binding assay method.

In one embodiment, the method is carried out within one week following fever onset in the subject.

In one embodiment, the method further comprises administering a DENV treatment regimen to the subject if the subject is indicated for a current DENV infection.

In one aspect, there is provided a method of evaluating a subject's suitability for a DENV vaccine, the method comprising: contacting a sample of the subject with an aptamer or the mixture of aptamers in the presence of a DENV protein; detecting a binding event at the aptamer(s); determining an immune history of the subject based on the binding event at the aptamer(s), wherein an absence of a binding event at any of the aptamer(s) is indicative of a past DENV infection in the subject; concluding the suitability of the subject for the DENV vaccine based on the immune history.

In one aspect, there is provided a kit for identifying a DENV infection in a subject, the kit comprising the aptamer or the mixture of aptamers.

In one embodiment, the kit further comprises a DENV protein.

In one aspect, there is provided a nucleic acid molecule comprising a sequence set out in the table below:

| | Sequence (L = Biotin-dT, x= dDs, d = Diol1-dPa, |
|---|---|
| SEQ ID NO. | y = Diol1-dPx, w = Diol1-dPa or Diol1-dPx) |
| 11 | CCCCAGACGGACTGGTGTxCTCGGxATGGCCGTCTGGGGCGCGLAGCG |
| 12 | GGCTGGTCCGxCTGGGAACAAGxGGCGGGAGGGAdGGGTGTGGGTGCGACAAGCGGACC |
| | AGCCCGCGLAGCG |
| 13 | CCGCTTGTCATCTAxCCTGGCCxTGTGGTACTGTAACGGCTGACAAGCGGCGCGLAGCG |
| 14 | CGGCGGAGACGTAACGCxTATCAAATCxAAACAGCTTAGGGTCCGCCGCGCGLAGCG |
| 15 | Biotin-TTTCGCACTCCATGATATGGTCTACTGAGCGAGACGATGCTGCTAAAxTACG |
| | CCGTGGTxACGAAGACAGACAAGCGGAGTAGTTAGACCGTGAAA |
| 16 | GCACTCCATGATATGGTCTACTGAGCGAGACGATGCTGCTAAAxTACGCCGTGGTxACG |
| | AAGACAGACAAGCGGAGTGTCGCGLAGCG |
| 17 | LGATATGGTCTACTGTGTGAxGTCCTACAATGGACTGGTGTxCTCGGxATGGCCATTGA |
| | CAAGCGGAGTAGTTAGACC |
| 18 | CAGACGGACTGGTGTxCTCGGxATGGCCGTCTGCGCGLAGCG |
| 19 | LTTTCGCACTCCATGATATGGTCTACTGGTCCGxCTGGGAACAAGxGGCGGGAGGGAdG |
| | GGTGTGGGTGCGACAAGCGGAGTAGTTAGACCGTCAAA |
| 20 | Biotin-TTTCGCACTCCATGATATGGTCTACTGGTCCGxCTGGGAACAAGxGGCGGGA |
| | GGGAyGGGTGTGGGTGCGACAAGCGGAGTAGTTAGACCGTCAAA |
| 21 | LTTTCGCACTCCATGATATGGTCTACTGGTCCGxCTGGGAACAAGxGGCGGGAGGGAdG |
| | GGTGTGGGTGCGACAAGCGGAGTAG |
| 22 | LCATGATATGGTCTACTGGTCCGxCTGGGAACAAGxGGCGGGAGGGAdGGGTGTGGGTG |
| | CGACAAGCGGAGTAG |
| 23 | GACGGTCTACTGGTCCGxCTGGGAACAAGxGGCGGGAGGGAdGGGTGTGGGTGCGACAA |
| | GCGGAGTAGTTAGACCGTCCGCGLAGCG |
| 24 | GGTCTACTGGTCCGxCTGGGAACAAGxGGCGGGAGGGAdGGGTGTGGGTGCGACAAGCG |
| | GAGTAGACCCGCGLAGCG |
| 25 | GGTCCGxCTGGGAACAAGxGGCGGGAGGGAdGGGTGTGGGTGCGACAAGCGGCGCGLAG |
| | CG |
| 26 | LGATATGGTCTACTGAAGTGTTGTCATCTAxCCTGGCCxTGTGGTACTGTAACGGCTGA |
| | CAAGCGGAGTAGTTAGACC |
| 27 | LGATATGGTCTACTGTGGCGCGAGGGAATCxACGCxTATCAAATAxAAACAGCTAATGA |
| | CAAGCGGAGTAGTTAGACC |
| 28 | LGATATGGTCTACTGAGGAGCGCATGTCGAGATACCAACCxCCATCCAATCxTTCTTGA |
| | CAAGCGGAGTAGTTAGACC |
| 29 | LTGATATGGTCTACTGACGCCGGGGCCCGTAxTCAGACGTATACxCATCAGGGCACATA |
| | CAAGCGGAGTAGTTAGACC |
| 30 | CGAGGCCCGTAxTCAGACGTATACxCATCAGGGCCTCGCGCGLAGCG |
| 31 | GGCAGCGCGTCGATTGxCCAATCTTAGCCAACCCAAAATTACAAGCGCTGCCCGCGLAG |
| | CG |
| 32 | GCTGCCTxGTACCAACCCCCTCCAATCxATTAGGCAGCCGCGLAGCG |
| 33 | CGTGCGACGAxGTCCAACCAGTCCCAATCxACAAGTCGCACGCGCGLAGCG |
| 34 | GCGGTCCGTGCxGTCGCCAATCCGTGdTCCAACCCCGACAAGCGGACCGCCGCGLAGCG |
| 35 | GCCCGCTTTCGxCCAACCCGTGdTCCAATCCCAGAAAGCGGGCCGCGLAGCG |
| 36 | CGCCCGTCAAGGxCTCCAATCCGTGdTCCAACCAGTTTTGACGGGCGCGCGLAGCG |
| 37 | GCCCGCGTGCTCAACCTTACCAATCTGxCACGCGGGCCGCGLAGCG |
| 38 | GCCCTGCGxGCTCAACCTTACCAATCTGxCACGCAGGGCCGCGLAGCG |
| 39 | LACTCCATGATATGGTCTACTGATAGTACTCCxGTTTAACTCTGAxACTTGACGTCCAT |
| | TCATAGACAAGCGGAGTAGTTAGACC |
| 40 | LGATATGGTCTACTGGGGCTTGGTCTTGCGTxTGCAGATTAACTTGCGTGCCAGTAAGA |
| | CAAGCGGAGTAGTTAGACC |
| 41 | LGATATGGTCTACTGTCTCAACGGTTGTCAAACGGxTATCACGGCxACACACCTGCGGA |
| | CAAGCGGAGTAGTTAGACC |
| 42 | CTCCGCTGTCAAACGGxTATCACGGCxACACACCTGCGGACAGCGGAGCGCGLAGCG |
| 43 | LGATATGGTCTACTGTCACAxATCGCCGTAAAGxCGAAGAGCTGCGGAATCTAAGGTGA |
| | CAAGCGGAGTAGTTAGACC |
| 44 | LGATATGGTCTACTGTATAATCCGCxTTCGTCATGTGGxTTGGATCTGGGTCTGGCAGA |
| | CAAGCGGAGTAGTTAGACC |
| 45 | LGATATGGTCTACTGCCCAAxCTTGTCTGTAAGGGxTTGGxTAGGGCTGGCAAAAAAGA |
| | CAAGCGGAGTAGTTAGACC |
| 46 | CGGCCGATGCTGCTAAAxTACGCCGTGGTxACGAAGACAGACAAGCGGAGTAGTTAGAC |
| | CGGCCGCGCGLAGCG |
| 47 | GCGCCAAAxTACGCCGTGGTxCGAAGACAGACAAGCGGAGTAGTTGGCGCCGCGLAGCG |
| 48 | GCACTCCGTCTACTGAGCGAGACGATGCTGCTAAAxTACGCCGTGGTxACGAAGACGGA |
| | GTGTCGCGLAGCG |
| 49 | GCACTCCGCTACTGAGCGAGACGATGCTGCTAAAxTACGCCGTGGTxACGAAGACAGCG |
| | GAGTGTCGCGLAGCG |
| 50 | GGCTGGTCCGACTGGGAACAAGxGGCGGGAGGGAdGGGTGTGGGTGCGACAAGCGGACC |
| | AGCCCGCGLAGCG |
| 51 | ACTGGTGTxCTCGGxATGG |
| 52 | TGGGAACAAGxGGCGGGAGGGAwGGGTGTGGGTGCGACAAG |
| 53 | TCTAxCCTGGCCxTGTGGTACTGTAACGGC |
| 54 | GACGTAACGCxTATCAAATCxAAACAGCT |
| 55 | ATGATATGGTCTACTGAGCGAGACGATGCTGCTAAAxTACGCCGTGGTxACGAAGACAG |
| | ACAAGC |
| 56 | GAGGGAATCxACGCxTATCAAATAxAAACAGCT |
| 57 | AAACGGxTATCACGGCxACACACCTGCG |

or; a sequence sharing at least 75% sequence identity thereto; or a sequence differing by one, two, three, four, five, six, seven, eight, nine or ten bases thereto; or portions thereof.

### Definitions

The term “aptamer” as used herein broadly refers to a nucleic acid molecule that is capable of binding with high affinity and specificity to a target molecule, in particular to a dengue virus protein. An “aptamer” not only includes nucleic acid molecules composed of natural bases, but also include those comprising unnatural or artificial bases, modified bases and/or nucleic acid analogs. An “aptamer” may also have modifications. Non-limiting examples of such modifications include: terminus modification to increase a stability of the molecule, functional group modification (e.g. amino, thiol, ethyl, diol), conjugation modification (e.g. biotinylation) and phosphorothioate bond modification.

The term “identifying” as used herein in relation to an infection is to be interpreted broadly to encompass determining a presence, an absence, an amount, a level of disease burden, a phase or a nature of the infection. For example, identifying a phase of the infection may comprise determining whether the infection is in a febrile phase, a critical phase or a recovery phase etc. For example, identifying a nature of the infection may comprise determining a serotype of the infection, determining whether the infection is a primary infection or a secondary or subsequent infection and/or determining whether the infection is a current infection or a past infection.

The term “treatment”, “treat” and “therapy”, and synonyms thereof as used herein refer to both therapeutic treatment and prophylactic or preventative measures, wherein the object is to prevent or slow down (lessen) a medical condition, which includes but is not limited to diseases (such as dengue infection), symptoms and disorders. A medical condition also includes a body's response to a disease or disorder, e.g. inflammation. Those in need of such treatment include those already with a medical condition as well as those prone to getting the medical condition or those in whom a medical condition is to be prevented.

The term “subject” as used herein includes patients and non-patients. The term “patient” refers to individuals suffering or are likely to suffer from a medical condition such as a flavivirus or dengue virus infection, while “non-patients” refer to individuals not suffering and are likely to not suffer from the medical condition. “Non-patients” include healthy individuals, non-diseased individuals and/or an individual free from the medical condition. The term “subject” includes humans and animals. Animals include murine and the like. “Murine” refers to any mammal from the family Muridae, such as mouse, rat, and the like.

The term “micro” as used herein is to be interpreted broadly to include dimensions from about 1 micron to about 1000 microns. When used as a unit prefix, 1 micro (μ) denotes a factor of 10⁻⁶.

The term “nano” as used herein is to be interpreted broadly to include dimensions less than about 1000 nm. When used as a unit prefix, 1 nano (n) denotes a factor of 10⁻⁹.

The term “particle” as used herein broadly refers to a discrete entity or a discrete body. The particle described herein can include an organic, an inorganic or a biological particle. The particle used described herein may also be a macro-particle that is formed by an aggregate of a plurality of sub-particles or a fragment of a small object. The particle of the present disclosure may be spherical, substantially spherical, or non-spherical, such as irregularly shaped particles or ellipsoidally shaped particles. The term “size” when used to refer to the particle broadly refers to the largest dimension of the particle. For example, when the particle is substantially spherical, the term “size” can refer to the diameter of the particle; or when the particle is substantially non-spherical, the term “size” can refer to the largest length of the particle.

The terms “coupled” or “connected” as used in this description are intended to cover both directly connected or connected through one or more intermediate means, unless otherwise stated.

The term “associated with”, used herein when referring to two elements refers to a broad relationship between the two elements. The relationship includes, but is not limited to a physical, a chemical or a biological relationship. For example, when element A is associated with element B, elements A and B may be directly or indirectly attached to each other or element A may contain element B or vice versa.

The term “adjacent” used herein when referring to two elements refers to one element being in close proximity to another element and may be but is not limited to the elements contacting each other or may further include the elements being separated by one or more further elements disposed therebetween.

The term “and/or”, e.g., “X and/or Y” is understood to mean either “X and Y” or “X or Y” and should be taken to provide explicit support for both meanings or for either meaning.

Further, in the description herein, the word “substantially” whenever used is understood to include, but not restricted to, “entirely” or “completely” and the like. In addition, terms such as “comprising”, “comprise”, and the like whenever used, are intended to be non-restricting descriptive language in that they broadly include elements/components recited after such terms, in addition to other components not explicitly recited. For example, when “comprising” is used, reference to a “one” feature is also intended to be a reference to “at least one” of that feature. Terms such as “consisting”, “consist”, and the like, may in the appropriate context, be considered as a subset of terms such as “comprising”, “comprise”, and the like. Therefore, in embodiments disclosed herein using the terms such as “comprising”, “comprise”, and the like, it will be appreciated that these embodiments provide teaching for corresponding embodiments using terms such as “consisting”, “consist”, and the like. Further, terms such as “about”, “approximately” and the like whenever used, typically means a reasonable variation, for example a variation of +/−5% of the disclosed value, or a variance of 4% of the disclosed value, or a variance of 3% of the disclosed value, a variance of 2% of the disclosed value or a variance of 1% of the disclosed value.

Furthermore, in the description herein, certain values may be disclosed in a range. The values showing the end points of a range are intended to illustrate a preferred range. Whenever a range has been described, it is intended that the range covers and teaches all possible sub-ranges as well as individual numerical values within that range. That is, the end points of a range should not be interpreted as inflexible limitations. For example, a description of a range of 1% to 5% is intended to have specifically disclosed sub-ranges 1% to 2%, 1% to 3%, 1% to 4%, 2% to 3% etc., as well as individually, values within that range such as 1%, 2%, 3%, 4% and 5%. The intention of the above specific disclosure is applicable to any depth/breadth of a range.

Additionally, when describing some embodiments, the disclosure may have disclosed a method and/or process as a particular sequence of steps. However, unless otherwise required, it will be appreciated that the method or process should not be limited to the particular sequence of steps disclosed. Other sequences of steps may be possible. The particular order of the steps disclosed herein should not be construed as undue limitations. Unless otherwise required, a method and/or process disclosed herein should not be limited to the steps being carried out in the order written. The sequence of steps may be varied and still remain within the scope of the disclosure.

Furthermore, it will be appreciated that while the present disclosure provides embodiments having one or more of the features/characteristics discussed herein, one or more of these features/characteristics may also be disclaimed in other alternative embodiments and the present disclosure provides support for such disclaimers and these associated alternative embodiments.

### DESCRIPTION OF EMBODIMENTS

Exemplary, non-limiting embodiments of a nucleic acid molecule, optionally an aptamer for dengue virus (DENV), and related methods, mixtures kits are disclosed hereinafter.

In various embodiments, there is provided a nucleic acid molecule that is capable of recognizing and/or binding to DENV or portions thereof. The nucleic acid molecule may be a polynucleotide or an oligonucleotide. The nucleic acid molecule may be composed of deoxyribonucleic acid (DNA) or ribonucleic acid (RNA). The nucleic acid molecule may comprise natural bases, unnatural or artificial bases, modified bases and/or nucleic acid analogs (e.g., containing base analogs, sugar analogs and/or a non-native backbone and the like). A nucleic acid molecule may also have modifications, for example, at its terminus or termini. The nucleic acid molecule may be single-stranded. In various embodiments, the nucleic acid molecule is capable of binding to DENV or portions thereof with high affinity and/or high specificity. No particular size is implied by the term “nucleic acid molecule”. In various embodiments, the nucleic acid molecule comprises an aptamer.

In various embodiments therefore, there is provided an aptamer for DENY. The aptamer may be an RNA-based aptamer, or an aptamer comprising ribonucleotide units or it may be a DNA-based aptamer, or an aptamer comprising deoxyribonucleotide units. In one embodiment, the aptamer comprises a DNA-based aptamer. Advantageously, a DNA-based aptamer may have increased stability. A DNA-based aptamer includes one with modification(s).

In various embodiments, the aptamer may comprise natural bases and/or unnatural bases (or artificial bases). A “natural base” refers to a naturally occurring base such as adenine (A), guanine (G), cytosine (C), thymine (T) (for a DNA-based aptamer) and uracil (U) (for an RNA-based aptamer). An “unnatural base” (or “artificial base”) refers to a base which is not a naturally occurring base. Non-limiting examples of an unnatural base (or an artificial base) include isoguanine (iG), isocytosine (iC), 2-amino-imidazo[1,2-a]-1,3,5-triazin-4(8H)-one (P), 6-amino-5-nitro-2(1H)-pyridone (Z), 7-(2-thienyl)-imidazo[4,5-b]pyridine (Ds), pyrrole-2-carbaldehyde (Pa), 2-nitropyrrole (Pn), 2-nitro-4-propynylpyrrole (Px), 7-(2,2′-bithien-5-yl)imidazo[4,5-b]pyridin-3-yl (Dss), 7-(2,2′,5′,2″-terthien-5-yl)imidazo[4,5-b]pyridin-3-yl (Dsss); 2-amino-6-(2-thienyl)purin-9-yl (s); 2-amino-6-(2,2′-bithien-5-yl)purin-9-yl (ss); 2-amino-6-(2,2′,5′,2″-terthien-5-yl)purin-9-yl (sss); 4-(2-thienyl)-pyrrolo[2,3-b]pyridin-1-yl (dDsa); 4-(2,2′-bithien-5-yl)-pyrrolo[2,3-b]pyridin-1-yl (Dsas); 4-[2-(2-thiazolyl)thien-5-yl]pyrrolo[2,3-b]pyridin-1-yl (Dsav); 4-(2-thiazolyl)-pyrrolo[2,3-b]pyridin-1-yl (dDva); 4-[5-(2-thienyl)thiazol-2-yl]pyrrolo[2,3-b]pyridin-1-yl (Dvas); 4-(2-imidazolyl)-pyrrolo[2,3-b]pyridin-1-yl (dDia), 6-methylisoquinoline-1 (2H)-thione (5SICS) or

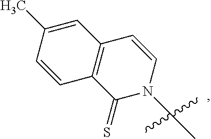 [SMILES: *n1ccc2cc(C)ccc2c1=S]

### TPT3 or

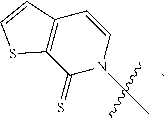 [SMILES: *n1ccc2ccsc2c1=S]

3-methoxynaphthalen-2-yl (NaM), 2-methoxy-4-methylphenyl (MMO2), derivatives thereof and the like. Examples of derivatives include dihydroxy derivatives such as diol-Px and diol-Pa (e.g. diol1-Px and diol1-Pa).

In some embodiments, examples of Px and Pa derivatives include

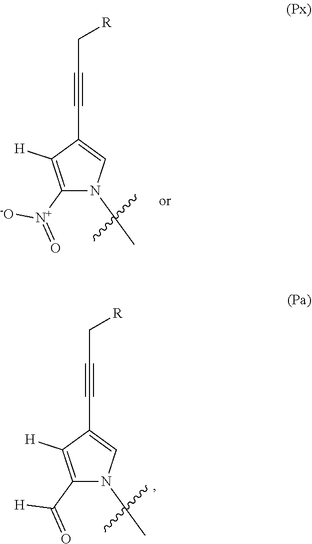

wherein R represents any moiety represented by the following formula:

| | —H |
|---|---|
| NH₂-C1- | —NH₂ |
| NH₂-C3- | —(CH₂)₂—NH₂ |
| NH₂-hx- | 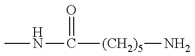 |
| N₃-C3- | —(CH₂)₂—N₃ |
| Eth-C4- | 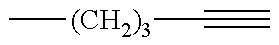 |
| Biotin- | 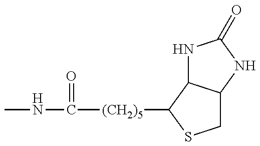 |
| Biotin-hx- | 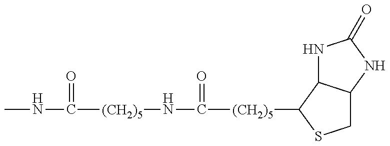 |
| Diol1- | 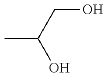 |
| Diol3O3- | 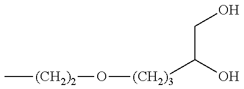 |
| FAM-hx- (FAM-) | 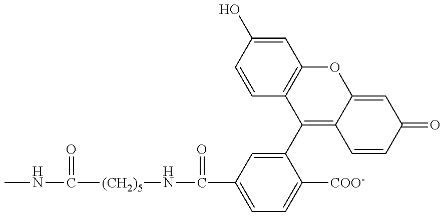 |
| TAMRA-hx- (TAMRA-) | 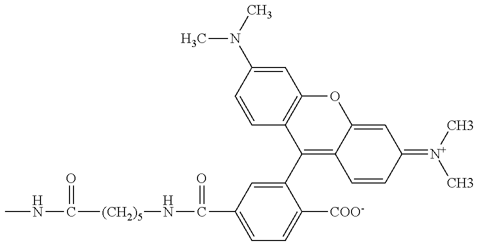 |
| Cy3-hx- (Cy3-) | 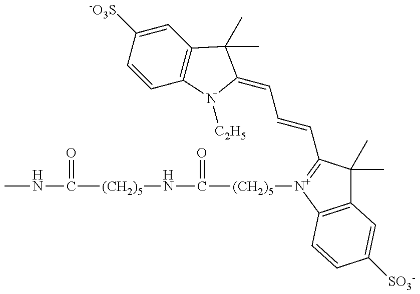 |
| Cy5-hx- (Cy5-) | 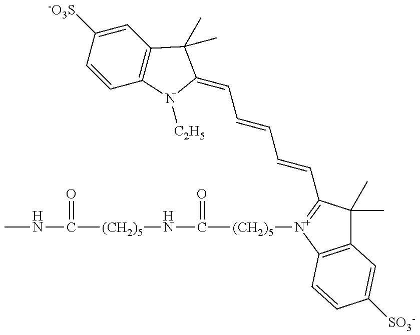 |
| Dig-hx- | 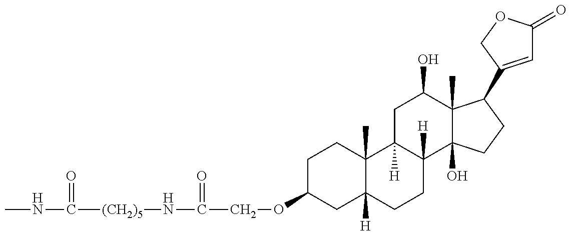 |
| DMP-hx- | 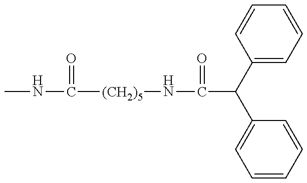 |
| | 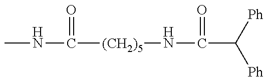 |
| NTP-hx- | 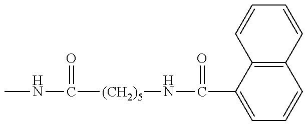 |
| BPh-hx- | 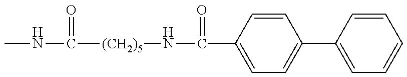 |
| HBP-hx- | 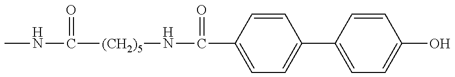 |
| furan-hx- | 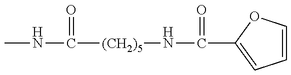 |
| Ph-hx- | 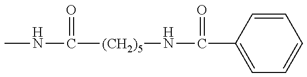 |
| thiophene- hx- | 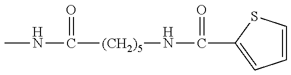 |
| pyrrole-hx- | 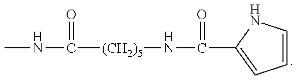 |

or

an amino-linked, an alkylamino-linked, a diol-linked, an aromatic group-linked, a fluorescent group-linked, a fluorescent dye-linked or a fluorophore-linked Pa or Px derivative; or

a Pa or Px derivative described in (i) Okamoto I, Miyatake Y, Kimoto M, Hirao I. High Fidelity, Efficiency and Functionalization of Ds-Px Unnatural Base Pairs in PCR Amplification for a Genetic Alphabet Expansion System. Okamoto I, Miyatake Y, Kimoto M, Hirao I. 2016 Nov. 18; 5(11):1220-1230. Epub 2016 Feb. 5. PMID: 26814421; (ii) Someya T, Ando A, Kimoto M, Hirao I. Site-specific labeling of RNA by combining genetic alphabet expansion transcription and copper-free click chemistry. Nucleic Acids Res. 2015 Aug. 18; 43(14):6665-76. doi: 10.1093/nar/gkv638. Epub 2015 Jun. 29. PMID:26130718; (iii) Ishizuka T, Kimoto M, Sato A, Hirao I. Site-specific functionalization of RNA molecules by an unnatural base pair transcription system via click chemistry. Chem Commun (Camb). 2012 Nov. 14; 48(88):10835-7. doi: 10.1039/c2cc36293g. Epub 2012 Oct. 3. PMID: 23032097; (iv) Morohashi N, Kimoto M, Sato A, Kawai R, Hirao I. Site-specific incorporation of functional components into RNA by an unnatural base pair transcription system. Molecules. 2012 Mar. 7; 17(3):2855-76. doi: 10.3390/molecules17032855. PMID: 22399139; (v) Yamashige R, Kimoto M, Takezawa Y, Sato A, Mitsui T, Yokoyama S, Hirao I. Highly specific unnatural base pair systems as a third base pair for PCR amplification. Nucleic Acids Res. 2012 March; 40(6):2793-806. doi: 10.1093/nar/gkr1068. Epub 2011 Nov. 24.

PMID: 22121213; (vi) Yamashige R, Kimoto M, Mitsui T, Yokoyama S, Hirao I. Monitoring the site-specific incorporation of dual fluorophore-quencher base analogues for target DNA detection by an unnatural base pair system. Org Biomol Chem. 2011 Nov. 7; 9(21):7504-9. doi: 10.1039/c1ob06118f. Epub 2011 Sep. 20. PMID:21935564. The contents of references (i) to (vi) are also incorporated by reference herein in their entirety.

Examples of Ds derivatives include

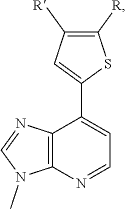

wherein R and R′ each independently represent any moiety represented by the following formula:

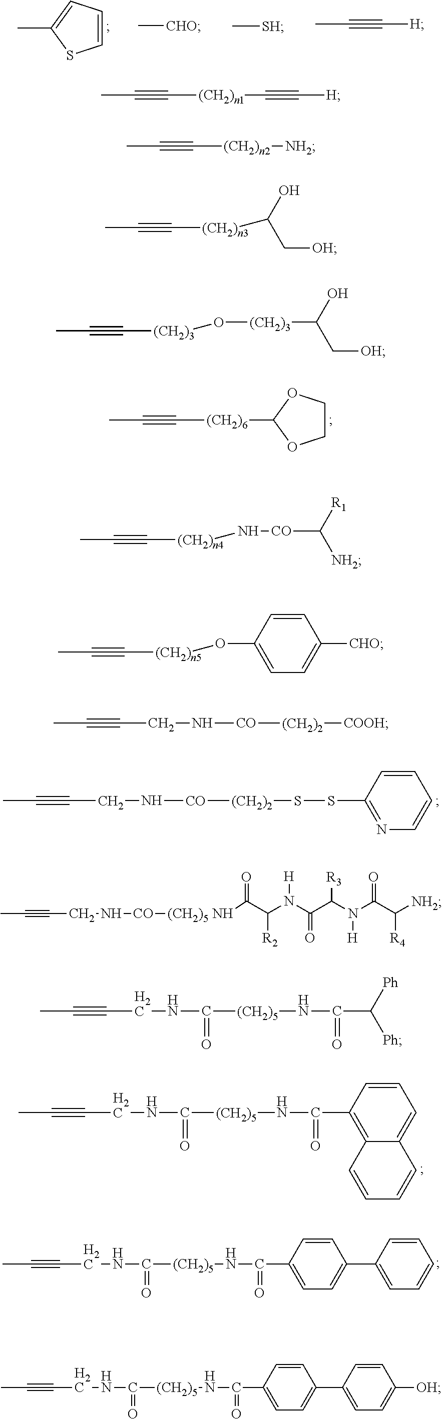

wherein n1=2 to 10; n2=1 or 3; n3=1, 6, or 9; n4=1 or 3; n5=3 or 6; R1=Phe (phenylalanine), Tyr (tyrosine), Trp (tryptophan), His (histidine), Ser (serine), or Lys (lysine); and R₂, R₃, and R₄=Leu (leucine), Leu, and Leu, respectively, or Trp, Phe, and Pro (proline), respectively.

In various embodiments, a derivative of a molecule or an unnatural base is structurally related to the molecule or the unnatural base. For example, the derivative may share a common structural feature, fundamental structure and/or underlying chemical basis with the molecule or the unnatural base. A derivative is not limited to one produced or obtained from the molecule or the unnatural base although it may be one produced or obtained from the molecule or the unnatural base. In some embodiments, the derivative is derivable, at least theoretically, from the molecule or the unnatural base through modification of the molecule or the unnatural base. In some embodiments, a derivative of a molecule or an unnatural base shares or at least retains to a certain extent a function, chemical property, biological property, chemical activity and/or biological activity associated with the molecule or the unnatural base. A skilled person will be able to identify, on a case by case basis and upon reading of the disclosure, the common structural feature, fundamental structure and/or underlying chemical basis of the molecule or the unnatural base that have to be maintained in the derivative to retain the function, chemical property, biological property, chemical activity, and/or biological activity. A skilled person will also be able to identify assays that can prove the retention of the function, chemical property, biological property, chemical activity, and/or biological activity. For example, a binding assay such as ELISA, EMSA, SPR and bio-layer interferometry (BLI) may be carried out to determine a binding property of an aptamer comprising an unnatural base derivative.

In various embodiments, the unnatural base is compatible with a polymerase, optionally a DNA polymerase. In various embodiments, the unnatural base is compatible with an amplification reaction such as polymerase chain reaction (PCR). In various embodiments, the unnatural base can form a base pair with another unnatural base. For example, Ds may base pair with Pn, Pa or Px. In some embodiments, the unnatural base forms a high fidelity pair with their complementary base in PCR. In various embodiments, the unnatural base is selected from members of an unnatural base pair system. For example, the unnatural base may comprise one or more members selected from Ds-Px pair, Ds-Pa pair, Ds-Pn pair, Dss-Px pair, Dss-Pa pair, Dss-Pn pair, iG-iC pair, P-Z pair, 5SICS-NaM pair, TPT3-NaM pair, 5SICS-MMO2 pair, derivatives thereof and the like.

In some embodiments, the unnatural base is hydrophobic. In some embodiments, the unnatural base is hydrophilic.

In various embodiments, the aptamer comprises from about one to about ten, or from about one to about five unnatural base(s). In various embodiments, the aptamer comprises at least about one, at least about two, at least about three, at least about four or at least about five unnatural base(s). In various embodiments, the aptamer comprises no more than about one, no more than about two, no more than about three, no more than about four or no more than about five unnatural base(s). In various embodiments, the aptamer comprises about one, about two, about three, about four, about five or more unnatural base(s).

The aptamer may comprise a hairpin structure or a stem-loop structure.

The aptamer may further comprise a bulge. In various embodiments, the aptamer comprises at least one hairpin structure or stem-loop structure and/or at least one bulge. In various embodiments, the unnatural base(s) resides in a loop structure of the aptamer. In various embodiments, the unnatural base(s) resides in a bulge of the aptamer. The bulge may or may not be in the loop structure. In various embodiments, the unnatural base(s) resides in a loop structure and/or a bulge of the aptamer. In some embodiments, from about one to about ten, or from about one to about five unnatural base(s) reside in a loop structure and/or a bulge of the aptamer. In some embodiments, at least about one, at least about two, at least about three, at least about four or at least about five unnatural base(s) resides in a loop structure and/or a bulge of the aptamer. In some embodiments, no more than about one, no more than about two, no more than about three, no more than about four or no more than about five unnatural base(s) resides in a loop structure and/or a bulge of the aptamer. In some embodiments, about one, about two, about three, about four, about five or more unnatural base(s) resides in a loop structure and/or a bulge of the aptamer. In some embodiments, all of the unnatural base(s) resides in a loop structure and/or a bulge of the aptamer. In some embodiments, a stem structure of the aptamer is devoid of an unnatural base. In some embodiments, the stem structure of the aptamer consists only of natural bases. In some embodiments, the loop structure and/or a bulge of the aptamer comprises unnatural base(s).

In some embodiments, the unnatural base in the aptamer does not have a binding partner. In some embodiments, the unnatural base in the aptamer does not form a base pair with a natural base. In some embodiments, the unnatural base forms a looped/buldged out region in the aptamer. A bulge may therefore be a portion of a nucleic acid or aptamer that has not been paired. Non-pairing may arise due to non-complementarity/mismatch base-pairing among natural bases, non-complementarity/mismatch base-pairing among unnatural bases, or non-complementarity/mismatch base-pairing among natural base(s) and unnatural base(s). In some examples, non-pairing arises due to the introduction of one or more unnatural base (e.g. in a region of natural bases) that does not form a base pair with natural bases. In some examples, a bulge includes from about 1 to about 20 bases, from about 1 to about 15 bases, from about 1 to about 10 bases or from about 1 to about 5 bases. In some examples, a bulge includes about 1, about 2, about 3, about 4, about 5, about 6, about 7, about 8, about 9, about 10, about 11, about 12, about 13, about 14, about 15, about 16, about 17, about 18, about 19 or about 20 bases.

In various embodiments, the number of bases in the aptamer is from about 20 to about 200, from about 20 to about 100, from about 30 to about 100, from about 40 to about 100, from about 40 to about 90, from about 40 to about 80, from about 40 to about 70 or from about 50 to about 60. Advantageously, embodiments of the aptamer having a short length or small size may have reduced unexpected interactions/toxicity, reduced cost of material/production and improved material quality assurance.

In various embodiments, the unnatural base is selected from the group consisting of: 7-(2thienyl)imidazo[4,5-b]pyridine (Ds); 7-(2,2′-bithien-5-yl)imidazo[4,5-b]pyridin-3-yl group (Dss); pyrrole2-carbaldehyde (Pa); diol-modified pyrrole2-carbaldehyde (diol-Pa); 2-nitro-4-propynylpyrrole (Px); diol-modified 2-nitro-4-propynylpyrrole (diol-Px); 7-(2,2′,5′,2″-terthien-5-yl)imidazo[4,5-b]pyridin-3-yl group (Dsss); 2-amino-6-(2-thienyl)purin-9-yl group (5); 2-amino-6-(2,2′-bithien-5-yl)purin-9-yl group (ss); 2-amino-6-(2,2′,5′,2″-terthien-5-yl)purin-9-yl group (sss); 4-(2-thienyl)-pyrrolo[2,3-b]pyridin-1-yl group (dDsa); 4-(2,2′-bithien-5-yl)-pyrrolo[2,3-b]pyridin-1-yl group (Dsas); 4-[2-(2-thiazolyl)thien-5-yl]pyrrolo[2,3-b]pyridin-1-yl group (Dsav); 4-(2-thiazolyl)-pyrrolo[2,3-b]pyridin-1-yl group (dDva); 4-[5-(2-thienyl)thiazol-2-yl]pyrrolo[2,3-b]pyridin-1-yl group (Dvas); 4-(2-imidazolyl)-pyrrolo[2,3-b]pyridin-1-yl group (dDia); derivatives thereof and combinations thereof. In various embodiments, the unnatural base is selected from the group consisting of: 7-(2thienyl)imidazo[4,5-b]pyridine (Ds), 7-(2,2′-bithien-5-yl)imidazo[4,5-b]pyridin-3-yl group (Dss), pyrrole2-carbaldehyde (Pa), diol-modified pyrrole2-carbaldehyde (diol-Pa), 2-nitro-4-propynylpyrrole (Px), diol-modified 2-nitro-4-propynylpyrrole (diol-Px), derivatives thereof and combinations thereof. In some embodiments, the aptamer comprises an unnatural base consisting of Ds, diol-Pa, diol-Px and derivatives thereof. In some embodiments, the aptamer comprises an unnatural base consisting of Ds. In some embodiments, the aptamer comprises more Ds (or derivatives thereof) than diol-Pa (or derivatives thereof) and/or diol-Px (or derivatives thereof).

In various embodiments, the aptamer comprising at least one unnatural base is capable of recognizing and/or binding to DENV, optionally a viral protein of DENV, optionally with high affinity and/or high specificity. In some embodiments, the aptamer comprising at least one unnatural base is capable of recognizing and/or binding to a non-structural protein of DENV, optionally with high affinity and/or high specificity. In some embodiments, the aptamer comprising at least one unnatural base is capable of recognizing and/or binding to a non-structural protein 1 (NS1) of DENV, optionally with high affinity and/or high specificity. In some embodiments, the aptamer comprising at least one unnatural base is capable of recognizing and/or binding to a serotype-specific NS1 of DENV, optionally with high affinity and/or high specificity. In various embodiments, the aptamer comprising at least one unnatural base is capable of forming a complex with DENV, a viral protein of DENV, a non-structural protein of DENV, NS1 of DENV and/or serotype-specific NS1 of DENV. For example, presence of an aptamer-DENV complex may be detected by an electrophoresis gel-mobility shift assay (EMSA). For example, high affinity and/or high specificity may be determined by surface plasmon resonance (SPR) analysis. For example, high affinity and/or high specificity may be determined by an enzyme-linked immunosorbent assay (ELISA). In various embodiments, the aptamer comprising at least one unnatural base is capable of recognizing and/or binding to DENV, a viral protein of DENV, a non-structural protein of DENV, NS1 of DENV and/or serotype-specific NS1 of DENV with higher affinity and/or higher specificity than their comparative sequences devoid of an unnatural base (e.g. sequences in which the unnatural base(s) is substituted with natural base(s) at the same position(s) or same relative position(s)).

In various embodiments, the aptamer is capable of distinguishing between DENV of different serotypes. For example, the aptamer is capable of distinguishing dengue fever serotype 1 (DEN1) from dengue fever serotype 2 (DEN2), dengue fever serotype 3 (DEN3) and/or dengue fever serotype 4 (DEN4). For example, the aptamer is capable of distinguishing DEN2 from DEN1, DEN3 and/or DEN4. For example, the aptamer is capable of distinguishing DEN3 from DEN1, DEN2 and/or DEN4. For example, the aptamer is capable of distinguishing DEN4 from DEN1, DEN2 and/or DEN3. For example, the aptamer is capable of recognizing and/or binding to DENV, a viral protein of DENV, a non-structural protein of DENV, NS1 of DENV of one serotype with substantially higher affinity than that/those of the other serotype(s). In some examples, the aptamer is incapable of recognizing and/or binding to or does not recognize and/or bind to DENV, a viral protein of DENV, a non-structural protein of DENV, NS1 of DENV of the other serotypes. In various embodiments, the aptamer is capable of binding specifically to a single serotype of DENV selected from the group consisting of serotype 1, serotype 2, serotype 3 and serotype 4.

Advantageously, embodiments of the aptamer possess high affinity for DENV, a viral protein of DENV, a non-structural protein of DENV, NS1 of DENV and/or serotype-specific NS1 of DENV. In various embodiments, the aptamer has a dissociation constant (K_{D}) of no more than about 14 nM, no more than about 13 nM, no more than about 12 nM, no more than about 11 nM, no more than about 10 nM, no more than about 9 nM, no more than about 8 nM, no more than about 7 nM, no more than about 6 nM, no more than about 5 nM, no more than about 4 nM, no more than about 3 nM, no more than about 2 nM or no more than about 1 nM. In various embodiments, the aptamer has a K_{D} of no more than about 800 pM, no more than about 600 pM, no more than about 400 pM, no more than about 300 pM, no more than about 250 pM, no more than about 200 pM, no more than about 190 pM, no more than about 180 pM, no more than about 170 pM, no more than about 160 pM, no more than about 150 pM, no more than about 140 pM, no more than about 130 pM, no more than about 120 pM, no more than about 110 pM, no more than about 100 pM, no more than about 90 pM, no more than about 80 pM, no more than about 70 pM, no more than about 60 pM, no more than about 50 pM, no more than about 40 pM, no more than about 30 pM, no more than about 20 pM or no more than about 10 pM, In one embodiment, the K_{D} of the aptamer for DENV is no more than 200 pM.

In various embodiments, the aptamer comprises a sequence set out in the Table 1 below:

TABLE 1

| SEQ ID NO. | Name | Sequence (L = Biotin-dT, x= dDs, d = Diol1-d Pa, y = Diol1-dPx) |
|---|---|---|
| 11 | D1-1-48h | CCCCAGACGGACTGGTGTxCTCGGxATGGCCGTCTGGGGCGCGLAGCG |
| 12 | D2-1d-72h | GGCTGGTCCGxCTGGGAACAAGxGGCGGGAGGGAdGGGTGTGGGTGCGACAAGCGGACCAGCCC |
| | | GCGLAGCG |
| 13 | D3-2-59h | CCGCTTGTCATCTAxCCTGGCCxTGTGGTACTGTAACGGCTGACAAGCGGCGCGLAGCG |
| 14 | D4-3-57h | CGGCGGAGACGTAACGCxTATCAAATCxAAACAGCTTAGGGTCCGCCGCGCGLAGCG |
| 15 | 1.9D1F1 | Biotin-TTTCGCACTCCATGATATGGTCTACTGAGCGAGACGATGCTGCTAAAxTACGCCGTG |
| | (isolate) | GTxACGAAGACAGACAAGCGGAGTAGTTAGACCGTGAAA |
| | | GCACTCCATGATATGGTCTACTGAGCGAGACGATGC |
| 16 | 19D1F1-3 | TGCTAAAxTACGCCGTGGTxACGAAGACAGACAAGCGGAGTGTCGCGLAGCG |
| 17 | D1-1-78 | LGATATGGTCTACTGTGTGAxGTCCTACAATGGACTGGTGTxCTCGGxATGGCCATTGACAAGC |
| | | GGAGTAGTTAGACC |
| 18 | D1-1-42h | CAGACGGACTGGTGTxCTCGGxATGGCCGTCTGCGCGLAGCG |
| 19 | D2-1d-97 | LTTTCGCACTCCATGATATGGTCTACTGGTCCGxCTGGGAACAAGxGGCGGGAGGGAdGGGTGT |
| | | GGGTGCGACAAGCGGAGTAGTTAGACCGTCAAA |
| 20 | D2-1y-96 | Biotin-TTTCGCACTCCATGATATGGTCTACTGGTCCGxCTGGGAACAAGxGGCGGGAGGGAy |
| | | GGGTGTGGGTGCGACAAGCGGAGTAGTTAGACCGTCAAA |
| 21 | D2-1d-84 | LTTTCGCACTCCATGATATGGTCTACTGGTCCGxCTGGGAACAAGxGGCGGGAGGGAdGGGTGT |
| | | GGGTGCGACAAGCGGAGTAG |
| 22 | D2-1d-74 | LCATGATATGGTCTACTGGTCCGxCTGGGAACAAGxGGCGGGAGGGAdGGGTGTGGGTGCGACA |
| | | AGCGGAGTAG |
| 23 | D2-1d-87h | GACGGTCTACTGGTCCGxCTGGGAACAAGxGGCGGGAGGGAdGGGTGTGGGTGCGACAAGCGGA |
| | | GTAGTTAGACCGTCCGCGLAGCG |
| 24 | D2-1d-77h | GGTCTACTGGTCCGxCTGGGAACAAGxGGCGGGAGGGAdGGGTGTGGGTGCGACAAGCGGAGTA |
| | | GACCCGCGLAGCG |
| 25 | D2-1d-61h | GGTCCGxCTGGGAACAAGxGGCGGGAGGGAdGGGTGTGGGTGCGACAAGCGGCGCGLAGCG |
| 26 | D3-2-78 | LGATATGGTCTACTGAAGTGTTGTCATCTAxCCTGGCCxTGTGGTACTGTAACGGCTGACAAGC |
| | | GGAGTAGTTAGACC |
| 27 | D4-3-78 | LGATATGGTCTACTGTGGCGCGAGGGAATCxACGCxTATCAAATAxAAACAGCTAATGACAAGC |
| | | GGAGTAGTTAGACC |
| 28 | D1-2-78 | LGATATGGTCTACTGAGGAGCGCATGTCGAGATACCAACCxCCATCCAATCxTTCTTGACAAGC |
| | | GGAGTAGTTAGACC |
| 29 | D1-3-78 | LTGATATGGTCTACTGACGCCGGGGCCCGTAxTCAGACGTATACxCATCAGGGCACATACAAGC |
| | | GGAGTAGTTAGACC |
| 30 | D1-3-47 | CGAGGCCCGTAxTCAGACGTATACxCATCAGGGCCTCGCGCGLAGCG |
| 31 | D1-5-61h | GGCAGCGCGTCGATTGxCCAATCTTAGCCAACCCAAAATTACAAGCGCTGCCCGCGLAGCG |
| 32 | D1-6-47h | GCTGCCTxGTACCAACCCCCTCCAATCxATTAGGCAGCCGCGLAGCG |
| 33 | D1-7-51h | CGTGCGACGAxGTCCAACCAGTCCCAATCxACAAGTCGCACGCGCGLAGCG |
| 34 | D2-2d-59h | GCGGTCCGTGCxGTCGCCAATCCGTGdTCCAACCCCGACAAGCGGACCGCCGCGLAGCG |
| 35 | D2-3d-52h | GCCCGCTTTCGxCCAACCCGTGdTCCAATCCCAGAAAGCGGGCCGCGLAGCG |
| 36 | D2-4d-56h | CGCCCGTCAAGGxCTCCAATCCGTGdTCCAACCAGTTTTGACGGGCGCGCGLAGCG |
| 37 | D2-5-46h | GCCCGCGTGCTCAACCTTACCAATCTGxCACGCGGGCCGCGLAGCG |
| 38 | D2-5-48h | GCCCTGCGxGCTCAACCTTACCAATCTGxCACGCAGGGCCGCGLAGCG |
| 39 | D3-1-85 | LACTCCATGATATGGTCTACTGATAGTACTCCxGTTTAACTCTGAxACTTGACGTCCATTCATA |
| | | GACAAGCGGAGTAGTTAGACC |
| 40 | D3-3-78 | LGATATGGTCTACTGGGGCTTGGTCTTGCGTxTGCAGATTAACTTGCGTGCCAGTAAGACAAGC |
| | | GGAGTAGTTAGACC |
| 41 | D4-1-78 | LGATATGGTCTACTGTCTCAACGGTTGTCAAACGGxTATCACGGCxACACACCTGCGGACAAGC |
| | | GGAGTAGTTAGACC |
| 42 | D4-1-57h | CTCCGCTGTCAAACGGxTATCACGGCxACACACCTGCGGACAGCGGAGCGCGLAGCG |
| 43 | D4-2-78 | LGATATGGTCTACTGTCACAxATCGCCGTAAAGxCGAAGAGCTGCGGAATCTAAGGTGACAAGC |
| | | GGAGTAGTTAGACC |
| 44 | D4-4-78 | LGATATGGTCTACTGTATAATCCGCxTTCGTCATGTGGxTTGGATCTGGGTCTGGCAGACAAGC |
| | | GGAGTAGTTAGACC |
| 45 | D4-5-78 | LGATATGGTCTACTGCCCAAxCTTGTCTGTAAGGGxTTGGxTAGGGCTGGCAAAAAAGACAAGC |
| | | GGAGTAGTTAGACC |
| 46 | 19D1F1-1 | CGGCCGATGCTGCTAAAxTACGCCGTGGTxACGAAGACAGACAAGCGGAGTAGTTAGACCGGCC |
| | | GCGCGLAGCG |
| 47 | 19D1F1-2 | GCGCCAAAxTACGCCGTGGTxCGAAGACAGACAAGCGGAGTAGTTGGCGCCGCGLAGCG |
| 48 | 19D1F1-4 | GCACTCCGTCTACTGAGCGAGACGATGCTGCTAAAxTACGCCGTGGTxACGAAGACGGAGTGTC |
| | | GCGLAGCG |
| 49 | 19D1F1-5 | GCACTCCGCTACTGAGCGAGACGATGCTGCTAAAxTACGCCGTGGTxACGAAGACAGCGGAGTG |
| | | TCGCGLAGCG |
| 50 | D1-1d-72h | GGCTGGTCCGACTGGGAACAAGxGGCGGGAGGGAdGGGTGTGGGTGCGACAAGCGGACCAGCCC |
| | (A) | GCGLAGCG |

or a sequence set out in Table 2 below:

TABLE 2

| | | Sequence |
|---|---|---|
| SEQ ID NO. | Name | (L = Biotin-dT, x= dDs, w = Diol1-dPa or Diol1-dPx) |
| 51 | D1-1-48h/ | ACTGGTGTxCTCGGxATGG |
| | D1-1-78/ | |
| | D1-1-42h | |
| | (core) | |
| 52 | D2-1d-72h/ | TGGGAACAAGxGGCGGGAGGGAwGGGTGTGGGTGCGACAAG |
| | D2-1d-97/ | |
| | D2-1d-84/ | |
| | D2-1d-74/ | |
| | D2-1d-87h/ | |
| | D2-1d-77h/ | |
| | D2-1d-61h/ | |
| | D2-1y-96 | |
| | (core) | |
| 53 | D3-2-59h/ | TCTAxCCTGGCCxTGTGGTACTGTAACGGC |
| | D3-2-78 | |
| | (core) | |
| 54 | D4-3-57h | GACGTAACGCxTATCAAATCxAAACAGCT |
| | (core) | |
| 55 | 19D1F1 | ATGATATGGTCTACTGAGCGAGACGATGCTGCTAAAxTACGCCGTGGTxACGAA |
| | (isolate)/ | GACAGACAAGC |
| | 19D1F1-3 | |
| | (core) | |
| 56 | D4-3-78 | GAGGGAATCxACGCxTATCAAATAxAAACAGCT |
| | (core) | |
| 57 | D4-1-78/ | AAACGGxTATCACGGCxACACACCTGCG |
| | D4-1-57h | |
| | (core) | |

or a sequence sharing at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% sequence identity thereto, or a sequence differing by about one, about two, about three, about four, about five, about six, about seven, about eight, about nine, about ten, about 11, about 12, about 13, about 14, about 15, about 16, about 17, about 18, about 19, about 20, about 21, about 22, about 23, about 24 or about 25 bases or lo nucleotides thereto, or portions thereof, optionally linear portions thereof. In some embodiments, the aptamer comprises a sequence differing by no more than about one, no more than about two, no more than about three, no more than about four, no more than about five, no more than about six, no more than about seven, no more than about eight, no more than about nine, no more than about ten, no more than about 11, no more than about 12, no more than about 13, no more than about 14, no more than about 15, no more than about 16, no more than about 17, no more than about 18, no more than about 19, no more than about 20, no more than about 21, no more than about 22, no more than about 23, no more than about 24 or no more than about 25 bases or nucleotides with a sequence in Table 1, or portions thereof, optionally linear portions thereof.

In some embodiments, the aptamer comprises one or more of a core sequences, stem region(s) and hairpin sequences e.g. mini-hairpin sequences. For example, Table 3 below denotes the core sequences, stem region(s) and hairpin sequences of some of the aptamers, with the core sequences indicated in bold, the stem region(s) indicated by a solid underline and the hairpin sequences indicated by a dotted underline.

TABLE 3

| SEQ | | |
|---|---|---|
| ID | | Sequence (L = Biotin-dT, x = dDs, d = Diol1-dPa, y = Diol1- |
| NO | Name | dPx) |
| 11 | D1-1-48h | 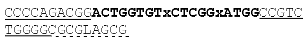 |
| 12 | D2-1d-72h | 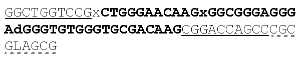 |
| 13 | D3-2-59h | 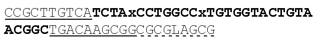 |
| 14 | D4-3-57h | 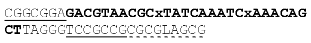 |
| 15 | 19D1F1 | Biotin-TTCGCACTCCATGATATGGTCTACTGAGCG |
| | (isolate) | AGACGATGCTGCTAAAxTACGCCGTGGTxACGAA |
| | | GACAGACAAGCGGAGTAGTTAGACCGTGAAA |
| 16 | 19D1F1-3 | GCACTCCATGATATGGTCTACTGAGCGAGACGAT |
| | | GCTGCTAAAxTACGCCGTGGTxACGAAGACAGAC |
| | | AAGCGGAGTGTCGCGLAGCG |
| 17 | D1-1-78 | LGATATGGTCTACTGTGTGAxGTCCTACAATGGAC |
| | | TGGTGTxCTCGGxATGGCCATTGACAAGCGGAGTA |
| | | GTTAGACC |
| 18 | D1-1-42h | 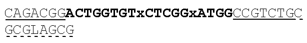 |
| 19 | D2-1d-97 | LTTTCGCACTCCATGATATGGTCTACTGGTCCGxCT |
| | | GGGAACAAGxGGCGGGAGGGAdGGGTGTGGGTG |
| | | CGACAAGCGGAGTAGTTAGACCGTCAAA |
| 20 | D2-1y-96 | Biotin-TTCGCACTCCATGATATGGTCTACTGGTCC |
| | | GxCTGGGAACAAGxGGCGGGAGGGAyGGGTGTG |
| | | GGTGCGACAAGCGGAGTAGTTAGACCGTCAAA |
| 21 | D2-1d-84 | LTTTCGCACTCCATGATATGGTCTACTGGTCCGxCT |
| | | GGGAACAAGxGGCGGGAGGGAdGGGTGTGGGTG |
| | | CGACAAGCGGAGTAG |
| 22 | D2-1d-74 | LCATGATATGGTCTACTGGTCCGxCTGGGAACAAG |
| | | xGGCGGGAGGGAdGGGTGTGGGTGCGACAAGCG |
| | | GAGTAG |
| 23 | D2-1d-87h | 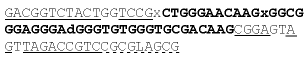 |
| 24 | D2-1d-77h | 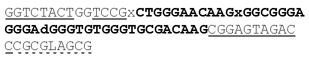 |
| 25 | D1-1d-61h | 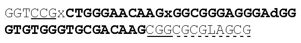 |
| 26 | D3-2-78 | LGATATGGTCTACTGAAGTGTTGTCATCTAxCCTG |
| | | GCCxTGTGGTACTGTAACGGCTGACAAGCGGAGT |
| | | AGTTAGACC |
| 27 | D4-3-78 | LGATATGGTCTACTGTGGCGCGAGGGAATCxACG |
| | | CxTATCAAATAxAAACAGCTAATGACAAGCGGAGT |
| | | AGTTAGACC |
| 41 | D4-1-78 | LGATATGGTCTACTGTCTCAACGGTTGTCAAACGG |
| | | xTATCACGGCxACACACCTGCGGACAAGCGGAGT |
| | | AGTTAGACC |
| 42 | D4-1-57h | 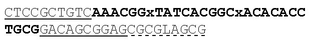 |

In various embodiments, the core sequences do not comprise hairpin sequences. In various embodiments, the hairpin sequences comprise CGCGLAGCG mini-hairpin sequences. In various embodiments, the hairpin sequences allow for stabilization and biotinylation of the aptamers without substantially affecting or negatively affecting the aptamer binding affinities. In some examples, the stem region(s) in the secondary structure at the 3′- and/or 5′-terminus may be replaced with other bases and/or base pairs without substantially affecting or negatively affecting the aptamer binding affinities.

In various embodiments, the aptamer that is specific to DEN1 comprises a sequence set out in Table 4 below.

TABLE 4

| | | Sequence |
|---|---|---|
| SEQ ID NO. | Name | (L = Biotin-dT, x= dDs, d = Diol1-d Pa, y = Diol1-dPx) |
| 11 | D11-48h | CCCCAGACGGACTGGTGTxCTCGGxATGGCCGTCTGGGGCGCGLAGCG |
| 15 | 19D1F1 | Biotin-TTTCGCACTCCATGATATGGTCTACTGAGCGAGACGATGCTGCTAAAx |
| | | TACGCCGTGGTxACGAAGACAGACAAGCGGAGTAGTTAGACCGTGAAA |
| 16 | 19D1F1-3 | GCACTCCATGATATGGTCTACTGAGCGAGACGATGCTGCTAAAxTACGCCGTGGT |
| | | xACGAAGACAGACAAGCGGAGTGTCGCGLAGCG |
| 17 | D1-1-78 | LGATATGGTCTACTGTGTGAxGTCCTACAATGGACTGGTGTxCTCGGxATGGCCA |
| | | TTGACAAGCGGAGTAGTTAGACC |
| 18 | D1142h | CAGACGGACTGGTGTxCTCGGxATGGCCGTCTGCGCGLAGCG |
| 28 | D1-2-78 | LGATATGGTCTACTGAGGAGCGCATGTCGAGATACCAACCxCCATCCAATCxTTC |
| | | TTGACAAGCGGAGTAGTTAGACC |
| 29 | D1-3-78 | LTGATATGGTCTACTGACGCCGGGGCCCGTAxTCAGACGTATACxCATCAGGGCA |
| | | CATACAAGCGGAGTAGTTAGACC |
| 30 | D1-3-47 | CGAGGCCCGTAxTCAGACGTATACxCATCAGGGCCTCGCGCGLAGCG |
| 31 | D1-5-61h | GGCAGCGCGTCGATTGxCCAATCTTAGCCAACCCAAAATTACAAGCGCTGCCCGC |
| | | GLAGCG |
| 32 | D1-6-47h | GCTGCCTxGTACCAACCCCCTCCAATCxATTAGGCAGCCGCGLAGCG |
| 33 | D1-7-51h | CGTGCGACGAxGTCCAACCAGTCCCAATCxACAAGTCGCACGCGCGLAGCG |
| 46 | 19D1F1-1 | CGGCCGATGCTGCTAAAxTACGCCGTGGTxACGAAGACAGACAAGCGGAGTAGTT |
| | | AGACCGGCCGCGCGLAGCG |
| 47 | 19D1F1-2 | GCGCCAAAxTACGCCGTGGTxCGAAGACAGACAAGCGGAGTAGTTGGCGCCGCGL |
| | | AGCG |
| 48 | 19D1F1-4 | GCACTCCGTCTACTGAGCGAGACGATGCTGCTAAAxTACGCCGTGGTxACGAAGA |
| | | CGGAGTGTCGCGLAGCG |
| 49 | 19D1F1-5 | GCACTCCGCTACTGAGCGAGACGATGCTGCTAAAxTACGCCGTGGTxACGAAGAC |
| | | AGCGGAGTGTCGCGLAGCG |
| 51 | D1-1-48h/ | ACTGGTGTxCTCGGxATGG |
| | D1-1-42h | |
| | (core) | |
| 55 | 19D1F1 | ATGATATGGTCTACTGAGCGAGACGATGCTGCTAAAxTACGCCGTGGTxACGAAG |
| | (isolate)/ | ACAGACAAGC |
| | 19D1F3-3 | |
| | (core) | |

In various embodiments, the aptamer that is specific to DEN2 comprises a sequence set out in Table 5 below.

TABLE 5

| | | Sequence (L = Biotin-dT, x= dDs, |
|---|---|---|
| SEQ NO. ID | Name | d = Diol1-dPa, y = Diol1-dPx, w = Diol1-dPa or Diol1-dPx) |
| 12 | D2-1d-72h | GGCTGGTCCGxCTGGGAACAAGxGGCGGGAGGGAdGGGTGTGGGTGCGACAAGCGGAC |
| | | CAGCCCGCGLAGCG |
| 19 | D2-1d-97 | LTTTCGCACTCCATGATATGGTCTACTGGTCCGxCTGGGAACAAGxGGCGGGAGGGAd |
| | | GGGTGTGGGTGCGACAAGCGGAGTAGTTAGACCGTCAAA |
| 20 | D2-1y-96 | Biotin-TTTCGCACTCCATGATATGGTCTACTGGTCCGxCTGGGAACAAGxGGCGGG |
| | | AGGGAyGGGTGTGGGTGCGACAAGCGGAGTAGTTAGACCGTCAAA |
| 21 | D2-1d-84 | LTTTCGCACTCCATGATATGGTCTACTGGTCCGxCTGGGAACAAGxGGCGGGAGGGAd |
| | | GGGTGTGGGTGCGACAAGCGGAGTAG |
| 22 | D2-1d-74 | LCATGATATGGTCTACTGGTCCGxCTGGGAACAAGxGGCGGGAGGGAdGGGTGTGGGT |
| | | GCGACAAGCGGAGTAG |
| 23 | D2-1d-87h | GACGGTCTACTGGTCCGxCTGGGAACAAGxGGCGGGAGGGAdGGGTGTGGGTGCGACA |
| | | AGCGGAGTAGTTAGACCGTCCGCGLAGCG |
| 24 | D2-1d-77h | GGTCTACTGGTCCGxCTGGGAACAAGxGGCGGGAGGGAdGGGTGTGGGTGCGACAAGC |
| | | GGAGTAGACCCGCGLAGCG |
| 25 | D2-1d-61h | GGTCCGxCTGGGAACAAGxGGCGGGAGGGAdGGGTGTGGGTGCGACAAGCGGCGCGLA |
| | | GCG |
| 34 | D2-2d-59h | GCGGTCCGTGCxGTCGCCAATCCGTGdTCCAACCCCGACAAGCGGACCGCCGCGLAGC |
| | | G |
| 35 | D2-3d-52h | GCCCGCTTTCGxCCAACCCGTGdTCCAATCCCAGAAAGCGGGCCGCGLAGCG |
| 36 | D2-4d-56h | CGCCCGTCAAGGxCTCCAATCCGTGdTCCAACCAGTTTTGACGGGCGCGCGLAGCG |
| 37 | D2-5-46h | GCCCGCGTGCTCAACCTTACCAATCTGxCACGCGGGCCGCGLAGCG |
| 38 | D2-5-48h | GCCCTGCGxGCTCAACCTTACCAATCTGxCACGCAGGGCCGCGLAGCG |
| 50 | D2-1d-72h | GGCTGGTCCGACTGGGAACAAGxGGCGGGAGGGAdGGGTGTGGGTGCGACAAGCGGAC |
| | (A) | CAGCCCGCGLAGCG |
| 52 | D2-1d-72h/ | TGGGAACAAGxGGCGGGAGGGAwGGGTGTGGGTGCGACAAG |
| | D2-1d-97/ | |
| | D2-1d-84/ | |
| | D2-1d-74/ | |
| | D2-1d-87h/ | |
| | D2-1d-77h/ | |
| | D2-1d-61h/ | |
| | D2-1y-96 | |
| | (core) | |

In various embodiments, the aptamer that is specific to DEN3 comprises a sequence set out in Table 6 below.

TABLE 6

| | | Sequence |
|---|---|---|
| SEQ ID NO. | Name | (L = Biotin-dT, x= dDs, d = Diol1-dPa, y = Diol1-dPx) |
| 13 | D3-2-59h | CCGCTTGTCATCTAxCCTGGCCxTGTGGTACTGTAACGGCTGACAAGCGGCGCG |
| | | LAGCG |
| 26 | D3-2-78 | LGATATGGTCTACTGAAGTGTTGTCATCTAxCCTGGCCxTGTGGTACTGTAQAC |
| | | GGCTGACAAGCGGAGTAGTTAGACC |
| 39 | D3-1-85 | LACTCCATGATATGGTCTACTGATAGTACTCCxGTTTAACTCTGAxACTTGACG |
| | | TCCATTCATAGACAAGCGGAGTAGTTAGACC |
| 40 | D3-3-78 | LGATATGGTCTACTGGGGCTTGGTCTTGCGTxTGCAGATTAACTTGCGTGCCAG |
| | | TAAGACAAGCGGAGTAGTTAGACC |
| 53 | D3-2-59h/ | TCTAxCCTGGCCxTGTGGTACTGTAACGGC |
| | D3-2-78 | |
| | (core) | |

In various embodiments, the aptamer that is specific to DEN4 comprises a sequence set out in Table 7 below.

TABLE 7

| | | Sequence |
|---|---|---|
| SEQ ID NO. | Name | (L = Biotin-dT, x= dDs, d = Diol1-dPa, y = Diol1-dPx) |
| 14 | D4-3-57h | CGGCGGAGACGTAACGCxTATCAAATCxAAACAGCTTAGGGTCCGCCGCGCGLA |
| | | GCG |
| 27 | D4-3-78 | LGATATGGTCTACTGTGGCGCGAGGGAATCxACGCxTATCAAATAxAAACAGCT |
| | | AATGACAAGCGGAGTAGTTAGACC |
| 41 | D4-1-78 | LGATATGGTCTACTGTCTCAACGGTTGTCAAACGGxTATCACGGCxACACACCT |
| | | GCGGACAAGCGGAGTAGTTAGACC |
| 42 | D4-1-57h | CTCCGCTGTCAAACGGxTATCACGGCxACACACCTGCGGACAGCGGAGCGGCGL |
| | | AGCG |
| 43 | D4-2-78 | LGATATGGTCTACTGTCACAxATCGCCGTAAAGxCGAAGAGCTGCGGAATCTAA |
| | | GGTGACAAGCGGAGTAGTTAGACC |
| 44 | D4-4-78 | LGATATGGTCTACTGTATAATCCGCxTTCGTCATGTGGxTTGGATCTGGGTCTG |
| | | GCAGACAAGCGGAGTAGTTAGACC |
| 45 | D4-5-78 | LGATATGGTCTACTGCCCAAxCTTGTCTGTAAGGGxTTGGxTAGGGCTGGCAAA |
| | | AAAGACAAGCGGAGTAGTTAGACC |
| 54 | D4-3-57h | GACGTAACGCxTATCAAATCxAAACAGCT |
| | (core) | |
| 56 | D4-3-78 | GAGGGAATCxACGCxTATCAAATAxAAACAGCT |
| | (core) | |
| 57 | D4-1-78/ | AAACGGxTATCACGGCxACACACCTGCG |
| | D4-1-57h | |
| | (core) | |

In various embodiments, the aptamer may be chemically modified.

In some embodiments, the aptamer comprises a chemical modification at its terminus or termini. In some embodiments, the aptamer comprises a chemical modification at its 3′ end. In some embodiments, the aptamer comprises a chemical modification at its 5′ end. In some embodiments, the chemical modification comprises introducing a mini-hairpin structure/sequence at a terminus, for example at the 3′ terminus. The mini-hairpin structure/sequence may be CGCGTAGCG, or a sequence differeing by no more than about one, no more than about two, or no more than about three nucleotides thereto. The mini-hairpin structure/sequence may increase a stability of the aptamer. For example, the mini-hairpin structure/sequence may protect the aptamer from rapid degradation by nucleases, particularly in a biological sample. Alternative or additional suitable chemical modification(s) may also be made to the aptamers. For example, the aptamer may also be capped at the 3′ or 5′ end with inverted deoxythymidine (idT) and/or locked nucleic acid (LNA) analogue.

In some embodiments, modification(s) may be made to the nucleotide unit(s) of the aptamer. For example, modification(s) may be made to the ribose or sugar moiety. In some examples, modification is made at 2′ position e.g. 2′-fluoro, 2′-methoxy, 2′-O-methyl and/or 2′-amino modification. In some examples, 2′-methoxy modification comprising deoxyribose modification at 2′-H and 2′-O-methyl modification comprising ribose modification at 2′-OH result in the same chemical structure. In some examples, the modification comprises a 4-thio-sugar modification. In some examples, the aptamer may be composed of or may comprise one or more nucleotide analogues such as peptide nucleic acid (PNA), locked nucleic acid (LNA), morpholino nucleotide, threose nucleic acid (TNA), glycol nucleic acid (GNA), arabinose nucleic acid (ANA), 2′-deoxy-2′-fluoro-β-D-arabinonucleic acid (2′ F-ANA), 2′-fluoroarabinose nucleic acid (FANA), 2′-deoxy-2′-fluororibonucleic acid (2′-F RNA or FRNA) cyclohexene nucleic acid (CeNA), anhydrohexitol nucleic acid (HNA), unlocked nucleic acid (UNA), (4′→6′) linked oligo 2′,3′-dideoxy-β-D-glucopyranose nucleic acid (homo-DNA or hDNA), xylonucleic acid (XyNA), deoxy-xylonucleic acids (dXyNA), aminoallyl uridine (aa-UTP), derivatives thereof and the like. In some examples, the aptamer may be composed of or may comprise L-ribose-based nucleotide.

In some embodiments, the modification comprises modification to a phosphate linkage part or a phosphodiester linkage. In some examples, the aptamer comprises one or more of a phosphorothioate linkage, a boranophosphate linkage, a methylphophonate, a phosphorthioate analogue, replacement to triazole linkage and the like.

In one example, the aptamer comprises phosphoramidite nucleotides.

In various embodiments, the modification(s) allows the aptamers to be stabilized against nucleic acid-cleaving/degrading enzymes such as nucleases or DNases. In various embodiments, the modification(s) increase the half-life of the aptamer e.g. in a biological sample such as human blood or serum. In various embodiments, the modification(s) allows the modified aptamers to gain desirable properties. In some embodiments, the modification(s) allows the modified aptamers to acquire desirable pharmacology and/or pharmacokinetic properties. In various embodiments, the modified aptamers are stable in the human body. In various embodiments, the modified aptamers possess desirable systemic clearance properties e.g. low glomerular filtration rate. In various embodiments, the modification(s) does not substantially reduce the affinity and/or specificity of the aptamers for DENV, a viral protein of DENV, a non-structural protein of DENV, NS1 of DENV and/or serotype-specific NS1 of DENV. In various embodiments, the affinity and/or specificity of the modified aptamers for DENV, a viral protein of DENV, a non-structural protein of DENV, NS1 of DENV and/or serotype-specific NS1 of DENV does not substantially differ or is not substantially reduced in comparison to that/those of the unmodified aptamers.

In various embodiments, the unnatural base(s) of the aptamer may also be modified. For example, the unnatural base(s) may be modified to have functional groups such as diol, azide, ethynyl and biotin. In one example, Pa is modified to diol-Pa. In one example, Px is modified to diol-Px. In one example, diol-Px is modified to diol-Pa. In various embodiments, an unnatural base comprising a diol group has enhanced affinity and/or specificity to DENV, a viral protein of DENV, a non-structural protein of DENV, NS1 of DENV and/or serotype-specific NS1 of DENV as compared to the unmodified unnatural base devoid of a diol group or a natural base variant. In some embodiments therefore, the unnatural base(s) comprise a diol group(s).

In various embodiments, the aptamer may be attached/linked/conjugated to one or more molecules. In some examples, functional group modification (e.g. amino, thiol, ethyl, diol etc.) facilitates chemical conjugation to the molecule (e.g. a label, a dye, a reporter, a carrier, a fluorophore, a solid support, a drug, polyethylene glycol (PEG), choresterol, albumin or other materials etc.) via linker. In one example, amino modification using NH₂-C6-dT is carried out to introduce a reactive amino group to the aptamer to facilitate chemical conjugation. In some embodiments, the aptamer may be conjugated to a carrier molecule or a reporter molecule. A carrier molecule may allow the aptamer conjugated thereto to attain desirable properties. A reporter molecule may allow the aptamer conjugated thereto to be detectable. An example of a carrier or reporter molecule is biotin. In one example, the thymidine in the mini-hairpin sequence CGCGAAGCG is used as the biotinylation site to generate a biotin-conjugated sequence CGCG(Biotin-T)AGCG which is then attached to the terminus of the aptamers, since the the Biotin-T position in the GAA tri-loop is acceptable to any natural bases (A,G,C adnT). In some examples, biotin-TEG-T (which comprises a tetraethylene glycol spacer arm) is used. Besides a biotinylated T, biotinylation may also be carried out at the 5′ end or 3′ end. In some examples, biotinylation facilitates immobilisation to streptavidin. Alternative or additional carrier molecule or reporter molecule may also be conjugated to the aptamer. In various embodiments, the conjugation does not substantially reduce the affinity and/or specificity of the aptamers for DENV, a viral protein of DENV, a non-structural protein of DENV, NS1 of DENV and/or serotype-specific NS1 of DENV.

Non-limiting examples of modification/modifiers include amino modifiers (e.g. amino modifier C6, amino modifier C12, amino modifier C6 dT, Uni-Link amino modifier etc.), biotinylation (e.g. biotin, biotin (azide), biotin dT, biotin-TEG, dual biotin, PC biotin, desthiobiotin-TEG etc.), thiol modifications (e.g. thiol modifier C3 S—S, dithiol, thiol modifier C6 S—S etc.), alkyne modifier (e.g. 5′ Hexynyl), 5-Octadiynyl dU etc.), Acrydite, adenylation, azide , azide (NHS Ester), cholesterol-TEG, digoxigenin, digoxigenin (NHS ester), I-Linker, fluorophore and dark quencher (e.g. fluorescein, Cy, rhodamine dye, Alexa Fluor dye, ATTO dye, IRDye, FAM, 6-FAM, 6-FAM (NHS ester), 6-FAM (fluorescein), fluorescein dT, Cy3, TAMRA, JOE, JOE (NHS ester), MAX, MAX (NHS ester), TET, Cy5.5, ROX, ROX (NHS ester), TYE 563, Yakima Yellow, HEX, TEX 615, TYE 665, TYE 705, Texas Red-X, Texas Red-X (NHS ester), Lightcycler 640, Lightcycler 640 (NHS Ester), Dy 750, Dy 750 (NHS ester), dark quencher, Iowa Black dark quencher, Iowa Black FQ, Iowa Black RQ, Black Hole Quencher-1, Black Hole Quencher-2, Dabcyl etc.), modified base modification (e.g. locked nucleic acid, 2′-O-methoxy-ethyl base (2′-MOE), 2′-O-methyl RNA base, fluoro base, 2-aminopurine, 5-Bromo dU, deoxyUridine, 2,6-Diaminopurine (2-Amino-dA), dideoxy-C, deoxylnosine, hydroxymethyl dC, inverted dT, iso-dG, iso-dC, inverted dideoxy-T, 5-methyl dC, 5-nitroindole etc.), phosphorylation modification, spacer modification (e.g. C3 spacer, hexanediol, 1′,2′-dideoxyribose (dSpacer), PC spacer, spacer 9, spacer 18 etc.), click chemistry modification (e.g. (i) 5′, Internal, or 3′ azide, (ii) 5′, Internal, or 3′ azide (NHS ester), (iii) 5′ hexynyl, (iv) 5′, Internal, or 3′ 5-Octadiynyl dU, (v) 5′, or Internal biotin, (vi) 5′, or internal biotin (azide), (vii) 5′, or Internal 6-FAM , (viii) 5′, or Internal 6-FAM (azide), (viiii) 5′, or Internal 5-TAMRA, (x) 5′, or Internal 5-TAMRA (azide) etc.), phosphorothioate bonds modification (e.g. phosphorothioated DNA bases, phosphorothioated RNA bases, phosphorothioated 2′-O-methyl bases, phosphorothioated Affinity Plus (locked nucleic acid) bases etc.) and the like. Embodiments of the aptamers may have some inhibitory effects on DENV. Embodiments of the aptamers may also show desirable pharmacology and/or pharmacokinetic properties (e.g. stability and/or low systemic clearance etc.).

In various embodiments, the aptamer is developed or selected from a SELEX (Systematic Evolution of Ligands by EXponential enrichment) method, optionally an ExSELEX (genetic alphabet Expansion for SELEX) method. In various embodiments, the aptamer is capable of recognizing and/or binding to a DENV protein, optionally a DEN-NS1 protein, sharing at least about 95%, at least about 95.5%, at least about 96%, at least about 96.1%, at least about 96.2%, at least about 96.3%, at least about 96.4%, at least about 96.5%, at least about 96.6%, at least about 96.7%, at least about 96.8%, at least about 96.9%, at least about 97%, at least about 97.1%, at least about 97.2%, at least about 97.3%, at least about 97.4%, at least about 97.5%, at least about 97.6%, at least about 97.7%, at least about 97.8%, at least about 97.9%, at least about 98%, at least about 98.1%, at least about 98.2%, at least about 98.3%, at least about 98.4%, at least about 98.5%, at least about 98.6%, at least about 98.7%, at least about 98.8%, at least about 98.9% or at least about 99% sequence identity/homology with a DENV protein, optionally a DEN-NS1 protein, used for selection in the SELEX or ExSELEX method. In various embodiments, the aptamer is capable of recognizing and/or binding to a DENV protein, optionally a DEN-NS1 protein, sharing more than about 95%, more than about 95.5%, more than about 96%, more than about 96.1%, more than about 96.2%, more than about 96.3%, more than about 96.4%, more than about 96.5%, more than about 96.6%, more than about 96.7%, more than about 96.8%, more than about 96.9%, more than about 97%, more than about 97.1%, more than about 97.2%, more than about 97.3%, more than about 97.4%, more than about 97.5%, more than about 97.6%, more than about 97.7%, more than about 97.8%, more than about 97.9%, more than about 98%, more than about 98.1%, more than about 98.2%, more than about 98.3%, more than about 98.4%, more than about 98.5%, more than about 98.6%, more than about 98.7%, more than about 98.8%, more than about 98.9% or more than about 99% sequence identity/homology with a DENV protein, optionally a DEN-NS1 protein, used for selection in the SELEX or ExSELEX method. In various embodiments, the aptamer is capable of recognizing and/or binding to a DENV protein, optionally a DEN-NS1 protein, having no more than about 15 amino acid difference, no more than about 14 amino acid difference, no more than about 13 amino acid difference, no more than about 12 amino acid difference, no more than about 11 amino acid difference, no more than about 10 amino acid difference, no more than about 9 amino acid difference, no more than about 8 amino acid difference, no more than about 7 amino acid difference, no more than about 6 amino acid difference, no more than about 5 amino acid difference, no more than about 4 amino acid difference, no more than about 3 amino acid difference, no more than about 2 amino acid difference or no more than about 1 amino acid difference with a DENV protein, optionally a DEN-NS1 protein, used for selection in the SELEX or ExSELEX method. In some embodiments, the DENV protein, optionally the DEN-NS1 protein, used for selection in the SELEX or ExSELEX method comprises a sequence selected from the group consisting of:

SEQ ID NO: 1:
DSGDVINWKGRELKCGSGIFVTNEVHTWTEQYKFQADSPKRLSAAIGKAW
EEGVCGIRSATRLENIMWKQISNELNHILLENDMKFTVVVGDVSGILAQG
KKMIRPQPMEHKYSWKSWGKAKIIGADVQNTTFIIDGPNTPECPDNQRAW
NIWEVEDYGFGIFTTNWILKLRDSYTQVCDHRLMSAAIKDSKAVHADMGY
WIESEKNETWKLARASFIEVKTCIWPKSHTLWSNGVLESEMIIPKIYGGP
ISQHNYRPGYFTQTAGPWHLGKLELDFDLCEGTTVVVDEHCGNRGPSLRT
TTVTGKTIHEWCCRSCTLPPLRFKGEDGCWYGMEIRPVKEKEENLVKSMV
SA;
SEQ ID NO: 2
DSGCVVSWKNKELKCGSGIFITDNVHTWTEQYKFQPESPSKLASAIQKAH
EEGICGIRSVTRLENLMWKQITPELNHILSENEVKLTIMTGDIKGIMQAG
KRSLRPQPTELKYSWKTWGKAKMLSTESHNQTFLIDGPETAECPNTNRAW
NSLEVEDYGFGVFTTNIWLKLKEKQDVFCDSKLMSAAIKDNRAVHADMGY
WIESALNDTWKIEKASFIEVKNCHWPKSHTLWSNGVLESEMIIPKNLAGP
VSQHNYRPGYHTQITGPWHLGKLEMDFDFCDGTTVVVTEDCGNRGPSLRT
TTASGKLITEWCCRSCTLPPLRYRGEDGCWYGMEIRPLKEKEENLVNSLV
TA;
SEQ ID NO: 3
DMGCVINWKGKELKCGSGIFVTNEVHTWTEQYKFQADSPKRLATAIAGAW
ENGVCGIRSTTRMENLLWKQIANELNYILWENNIKLTVVVGDTLGVLEQG
KRTLTPQPMELKYSWKTWGKAKIVTAETQNSSFIIDGPNTPECPSASRAW
NVWEVEDYGFGVGTTNIWLKLREVYTQLCDHRLMSAAVKDERAVHADMGY
WIESQKNGSWKLEKASLIEVKTCTWPKSHTLWTNGVLESDMIIPKSLAGP
ISQHNYRPGYHTQTAGPWHLGKLELDFNYCEGTTVVITESCGTRGPSLRT
TTVSGKLIHEWCCRSCTLPPLRYMGEDGCWYGMEIRPISEKEENMVKSLV
SA;
SEQ ID NO: 4
DMGCVASWSGKELKCGSGIFVVDNVHTWTEQYKFQPESPARLASAILNAH
KDGVCGIRSTTRLENVMWKQITNELNYVLWEGGHDLTVVAGDVKGVLTKG
KRALTPPVSDLKYSWKTWGKAKIFTPEARNSTFLIDGPDTSECPNERRAW
NSLEVEDYGFGMFTTNIWMKFREGSSEVCDHRLMSAAIKDQKAVHADMGY
WIESSKNQTWQIEKASLIEVKTCLWPKTHTLWSNGVLESQMLIPKSYAGP
FSQHNYRQGYATQTVGPWHLGKLEIDFGECPGTTVTIQEDCDHRGPSLRT
TTASGKLVTQWCCRSCTMPPLRFLGEDGCWYGMEIRPLSEKEENMVKSQV
TA;
SEQ ID NO: 5:
DSGCVINWKGRELKCGSGIVFTNEVHTWTEQYKFQADSPKRLLSAAIGKA
WEEGVCGIRSATRLENIMWKQISNELNHILLENDMKFTVVVGDANGILTQ
GKKMIRPQPMEHKYSWKSWGKAKIIGADTQNTTFIIDGPDTPECPDDQRA
WNIWEVEDYGFGVFTTNIWLKLRDSYTQMCDHRLMSAAIKDSKAVHADMG
YWIESEKNETWKLARASFIEVKTCIWPRSHTLWSNGVLESEMIIPKIYGG
PISQHNYRPGYFTQTAGPWHLGKLELDFNLCEGTTVVVDEHCGNRGPSLR
TTTVTGKIIHEWCCRSCTLPPLRFRGEDGCWYGMEIRPVKEKEENLVRSM
VSA;
SEQ ID NO: 6:
DSGCVINWKGRELKCGSGIFVTNEVHTWTEQYKFQADSPKRLSAAIGKAW
EEGVCGIRSATRLENIMWKQISNELNHILLENDMKFTVVVGDVAGILAQG
KKMIRPQPMEKHYSWKSWGKAKIIGADVQNTTFIIDGPNTPECPDDQRAW
NIWEVEDYGFGIFTTNIWLKLRDSYTQVCDHRLMSAAIKDSKAVHADMGY
WIESEKNETWKLARASFIEVKTCIWPKSHTLWSNGVLESEMIIPKIYGGP
ISQHNYRPGYFTQTAGPWHLGKLELDFDLCEGTTVVVDEHCGNRGPSLRT
TTVTGKIIHEWCCRSCTLPPLRFRGEDGCWYGMEIRPVKEKEENLVKSMV
SA;

a sequence thereof comprising a histidine tag at the C-terminal (e.g. HHHHHH or HHHHHHHHHH);

a sequence sharing at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% sequence identity thereto;

parts thereof; and

combinations thereof.

In some embodiments, the DENV protein, optionally the DEN-NS1 protein, used for selection in the SELEX or ExSELEX method comprises a combination of sequences selected from SEQ ID Nos. 1, 2, 3, 4 and 5.

In some embodiments therefore, the aptamer is capable of distinguishing between DENV variants, optionally DEN-NS1 variants, within a single serotype. In one example, the aptamer is capable of recognizing and/or binding to a DEN-NS1 protein sharing more than 96.3% sequence identity/homology with a DEN-NS1 protein used for selection in the SELEX or ExSELEX method but not a DEN-NS1 variant, which can be of the same serotype, having a lower sequence identity/homology. In one example, the aptamer is capable of recognizing and/or binding to a DEN-NS1 protein sharing more than 96.3% sequence identity/homology with SEQ ID NO. 1, but not DEN-NS1 variants having a lower sequence identity/homology. In one example, the aptamer is capable of recognizing and/or binding to a DEN-NS1 protein sharing about 98.9% sequence identity/homology with SEQ ID NO. 1. or a DEN-NS1 protein of SEQ ID NO: 6.

In various embodiments, there is provided a mixture/combination of aptamers that are specific to different serotypes, the mixture comprising at least two, at least three, or at least four of the aptamers. For example, the mixture/combination may comprise an aptamer that is specific to DEN1, an aptamer that is specific to DEN2, an aptamer that is specific to DEN3 and an aptamer that is specific to DEN4. For example, the mixture/combination may comprise any three of the following: an aptamer that is specific to DEN1, an aptamer that is specific to DEN2, an aptamer that is specific to DEN3 and an aptamer that is specific to DEN4. For example, the mixture/combination may comprise any two of the following: an aptamer that is specific to DEN1, an aptamer that is specific to DEN2, an aptamer that is specific to DEN3 and an aptamer that is specific to DEN4. In some embodiments, the mixture/combination of aptamers comprises one or more of SEQ ID NO: 11 (CCCCAGACGGACTGGTGTxCTCGGxATGGCCGTCTGGGGCGCGLAGCG) SEQ ID NO: 12 (GGCTGGTCCGxCTGGGAACAAGxGGCGGGAGGGAdGGGTGTGGGTGCG ACAAGCGGACCAGCCCGCGLAGCG), SEQ ID NO: 13 (CCGCTTGTCATCTAxCCTGGCCxTGTGGTACTGTAACGGCTGACAAGCGG CGCGLAGCG), SEQ ID NO: 14 (CGGCGGAGACGTAACGCxTATCAAATCxAAACAGCTTAGGGTCCGCCGCG CGLAGCG), SEQ ID NO: 15 (ITTTCGCACTCCATGATATGGTCTACTGAGCGAGACGATGCTGCTAAAxTA CGCCGTGGTxACGAAGACAGACAAGCGGAGTAGTTAGACCGTGAAA) and SEQ ID NO: 16 (GCACTCCATGATATGGTCTACTGAGCGAGACGATGCTGCTAAAxTACGCC GTGGTxACGAAGACAGACAAGCGGAGTGTCGCGLAGCG). In various embodiments, the mixture/combination allows for the detection with few amino-acid sequence mutations among dengue NS1 variants beyond serotype identification.

Embodiments of the aptamer or mixture may be used detecting a presence of DENV, a viral protein of DENV, a non-structural protein of DENV, NS1 of DENV and/or serotype-specific NS1 of DENV. Embodiments of the aptamer or mixture may be used detecting a presence of DENV, a viral protein of DENV, a non-structural protein of DENV, NS1 of DENV of a particular serotype.

In various embodiments, there is provided a method of identifying a DENV infection in a subject, the method comprising contacting a sample of the subject with the aptamer or the mixture of aptamers. In some embodiments, the method further comprises detecting a binding event at the aptamer(s). In various embodiments therefore, there is provided a method of identifying a DENV infection in a subject, the method comprising: contacting a sample of the subject with the aptamer or the mixture of aptamers and detecting a binding event at the aptamer(s). The method may be a method of identifying a current DENV infection, a method of identifying a past DENV infection and/or a method of identifying a characteristic/nature of a DENV infection (e.g. whether primary, secondary or further infection, serotype identification, disease burden etc.).

In various embodiments, the method may be implemented in the form of a binding assay. In various embodiments, the method may be in the form of a “sandwich” assay, where a first binder or capturing agent (e.g. an aptamer) serves to capture a target analyte (e.g. a DENV protein) and a second binder or detector agent (e.g. an anti-DENV) is used to detect the captured target analyte. Examples of such assays include enzyme immunoassays (EIA)/enzyme-linked immunosorbent assay (ELISA), enzyme-linked aptamers assays (ELAA) /enzyme-linked oligonucleotide assay (ELONA), radioimmunoassay RIA, strip assays, lateral flow assays (LFA), bio-layer interferometry (BLI), the detection through Surface plasmon resonance (SPR), colorimetric changes using gold nanoparticle aggregations, voltammetric, and electrochemical techniques and the like. The suitability of the specific assay to be used would be within the purview of the skill of the person skilled in the art. In some embodiments, the aptamer is immobilized on a solid support, optionally with an anti-DENV being used as the detector agent. In some embodiments, the aptamer is used as the detector agent, optionally with an anti-DENV being immobilized on a solid support. In some embodiments therefore, the detecting step comprises adding a detecting agent to detect a binding event at the aptamer(s).

In various embodiments, the method comprises an ELISA method, such as, but is not limited to direct ELISA, indirect ELISA, competitive ELISA, sandwich ELISA, and the like. In some embodiments, the method comprises a sandwich ELISA method. The sandwich ELISA may be competitive or non-competitive. In some examples, the aptamer may be labelled, for example with biotin, and may be bound to a solid phase e.g. a bead, a surface of a well or other containment body, a chip or a strip, for capturing any DENV protein and an antibody of the DENV protein is used for detecting any captured DENV protein. In some examples, a first antibody of the DENV protein is used as a primary detector agent and a second antibody against the first antibody is used as a secondary detector agent. The detector agent may be labelled, for example, with a dye, radioisotope or a reactive or catalytically active moiety. In one example, the detector agent is labelled with horseradish peroxidase (HRP) and tetramethylbenzidine (TMB) substrate may be added for visualization. It will be appreciated that other suitable labels and substrates may also be used. In some examples, a plate (e.g. microplate, microtiter plate etc.) coated with streptavidin is used for immobilising the biotin-labelled aptamer, which is then used for capturing a DENV protein, and a labelled detector agent (or a primary detector agent and a labelled secondary detector agent) is used for detection. The detector agent may be an anti-immunoglobulin such as anti-IgG (e.g. IgG1, IgG2, IgG3 or IgG4), anti-IgM or anti-IgA. In some examples, the anti-immunoglobulin comprises anti-human IgM, anti-goat IgG, anti-rabbit IgG, and/or anti-mouse IgG. When a primary detector is labelled with biotin, the secondary agent may be an anti-biotin antibody or streptavidin.

In some embodiments, a binding event at any of the aptamer(s) is indicative of a current DENV infection in the subject. In some examples, a binding event is indicated by a chemiluminescence or colorimetric signal/readout/output e.g. resulting from HRP conversion of TMB. In some embodiments, where the aptamer comprises an aptamer that is specific to or that binds specifically to a single DENV serotype, the binding event is indicative of a current DENV infection of said serotype in the subject. For example, where the aptamer is an aptamer specific to DEN1, detection of a binding event at the aptamer (e.g. observation of a colorimetric output in a HRP-TMB system) when the aptamer is contacted with a subject's sample is indicative that the subject has DEN1-protein in his sample, and therefore indicative that the subject has a current DEN1 infection. In this example, the DEN1-specific aptamer immobilised on a solid support, when contacted with the subject's sample, captures any DEN1-protein in the subject sample. The subsequent addition of a detector agent against the DEN1-protein then binds to any captured DEN1-protein. The detector agent gives a colorimetric output, thereby indicating the presence of captured DEN1 protein, and therefore the presence of DEN1 infection in the subject. In some embodiments therefore, where the method is a method of identifying a current DENV infection in the subject, a binding event at any of the aptamer(s) is indicative of a current DENV infection in the subject, optionally wherein the bound aptamer is specific to single DENV serotype, the binding event is indicative of a current DENV infection of said serotype in the subject.

Without wishing to be bound by theory, it is believed that virus-related materials such as the envelope protein and non-structural protein 1 (NS1) are detectable in a febrile phase or any early phase of DENV. Thus, in some embodiments, the binding event is indicative of a current DENV infection in its febrile phase or early phase. In some embodiments, the method is carried out within one week following fever onset in a subject or during a febrile or early phase of DENV infection.

In one example, the method comprises a non-competitive binding assay method. In one example, the method comprises a non-competitive ELISA method. In one example, the method comprises direct detection of DENV protein, optionally DENV-NS1 protein, in the sample.

In some embodiments, where the subject is indicated for a current DENV infection, the method further comprises contacting a sample of the subject with the aptamer or the mixture of aptamers in the presence of a DENV protein, and detecting a binding event at the aptamer(s). In some embodiments, the sample is pre-incubated with the DENV protein and then contacted with the aptamer or the mixture of aptamers.

Without wishing to be bound by theory, it is believed that DENV antigen binding protein, such as anti-DENV IgG, is generated by a subject's body in response to DENV infection. The serotype-specific DENV antigen binding protein may become detectable a number of days after illness onset, for example, one week after fever onset, and remain detectable after recovery. Thus, the detection of a serotype-specific DENV antigen binding protein in subject's sample during an early phase or febrile phase of a current DENV infection (for example, within one week after illness onset) may be indicative that the subject has a past DENV infection. Without wishing to be bound by theory, it is further believed that some of the DENV antigen binding protein generated, for example the anti-DENV IgG generated, are serotype-specific. For example, it is believed that a DENV antigen binding protein generated in response to a DEN1 infection is different from an DENV antigen binding protein generated in response to a DEN2, DEN3 or DEN4 infection. It is also believed that a DENV antigen binding protein generated in response to a DEN1 infection would only recognize and/or bind to a DEN1 protein, for example a DEN1-NS1 protein, and not or less other proteins belonging to DENV of a different serotype, for example, a DEN2-NS1 protein, a DEN3-NS1 protein and a DEN4-NS1 protein.

Without wishing to be bound by theory, it is believed that IgG production continues throughout life after a DENV infection, and thus IgG detection may enable the identification of past infections. In various embodiments therefore, the method comprises detecting the presence of anti-DENV IgG in a subject's sample.

In one example, where a subject is indicated for a current DEN1 infection, a second sample, which can be the same type of sample at the previous sample, can be collected from the subject and contacted with a mixture of aptamers comprising aptamers that are specific to DEN2, DEN3 and DEN4 in the presence of DEN2-NS1, DEN3-NS1 and DEN4-NS1 proteins to determine whether the subject has any DEN2, DEN3 and/or DEN4 past infections. The DEN2-NS1, DEN3-NS1 and DEN4-NS1 protein would normally bind to the DEN2 specific aptamer, the DEN3 specific aptamer and the DEN4 specific aptamer respectively. However, any anti-DEN2-NS1 IgG, anti-DEN3-NS1 IgG and/or anti-DEN4-NS1 IgG present in the sample may bind to the DEN2-NS1, DEN3-NS1 and/or DEN4-NS1 protein respectively and thereby inhibit the latter's binding to the DEN2 specific aptamer, the DEN3 specific aptamer and the DEN4 specific aptamer respectively. Thus, the absence of a binding event at any of the DEN2 specific aptamer, the DEN3 specific aptamer and the DEN4 specific aptamer may be indicative that the subject has anti-DEN2-NS1 IgG, anti-DEN3-NS1 IgG and/or anti-DEN4-NS1 IgG in his/her body, and hence indicative that the subject was previously infected with DEN2, DEN3 and/or DEN4. In some examples, a binding activity at the aptamer may be measured by a signal/output/readout e.g. a colorimetic signal/output/readout. In some examples, an intensity difference in the signal/output/readout associated with the different aptamers gives an indication of the probable serotype of previous infection in the sample.

In various embodiments therefore, an absence of a binding event at any of the aptamer(s) is indicative that the subject has past/primary DENV infection(s). In various embodiments, an absence of a binding event at any of the aptamer(s) in the sample which current infection was confirmed by the other method(s) is indicative that the current DENV infection is a secondary or further DENV infection.

In various embodiments therefore, where the subject is indicated for a current DENV infection, the method further comprises contacting a sample of the subject with the aptamer or the mixture of aptamers in the presence of a DENV protein, and detecting a binding event at the aptamer(s), wherein an absence of a binding event at any of the aptamer(s) is indicative that the current DENV infection is a secondary or further DENV infection, optionally wherein the unbound aptamer(s) is specific to a DENV serotype and the absence of the binding event(s) is indicative of a past DENV infection of said serotype(s) in the subject.

In various embodiments, the contacting step comprises contacting different amounts/volumes/concentrations of the subject's sample with the mixture of aptamers in the presence of a fixed amount/volume/concentration of the DENV proteins. An output/readout e.g. a colorimetic readout, indicative of a binding activity between each amount/volume/concentration of the sample with the DENV proteins, may be measured. The outputs/readouts (which can be transformed into values) may be plotted against the amounts/volumes/concentrations of the sample to obtain a graph. A point on the graph (e.g. the amount/volume/concentration of samples required to give a certain output/readout) may be used as a basis of comparison across the results obtained for each aptamer of the mixture of aptamers to obtain a relative binding activity of a sample for each aptamer of the mixture of aptamers. In some embodiments, the relative binding activity comprises a relative IgG activity of the sample.

As may be appreciated, a secondary or subsequent DENV infection of a different serotype of that of the primary infection may be more severe. For example, a secondary DENV infection is the greatest risk factor for severe disease such as Dengue Hemorrhagic Fever and Dengue Shock Syndrome. Without wishing to be bound by theory, it is believed that antibody-dependent enhancement (ADE) may contribute to severe dengue. Advantageously, embodiments of the method allowing for the determination whether a current DENV infection is a secondary or subsequent infection can facilitate clinical and treatment decisions. For example, a subject indicated to be suffering from a secondary DENV infection may be more closely monitored for development of severe dengue. Early medical care may be provided to the subject if needed, thereby reducing a risk of severe disease progression or fatality.

In one example, the method comprises a competitive binding assay method. In one example, the method comprises a competitive ELISA method. In one example, the method comprises competitive DENV antigen binding protein detection, optionally competitive anti-DENV IgG detection, further optionally anti-DENV-NS1 IgG detection.

In various embodiments, the different serotype-specific aptamers may be disposed or immobilised on different substrates, or they may be disposed or immobilised on the same substrate. For example, each serotype-specific aptamer may be immobilised separately on the supports (e.g. in different wells) as a capture agent. For example, four different serotype-specific aptamers may be immobilised on a single solid support (e.g. in a single well) as capture agents. In the latter example, different detection systems, for example, different colorimetric detection systems, may be employed to distinguish between the binding event(s) at the different serotype-specific aptamers. The four different serotype-specific aptamers may also be disposed at distinct/different/non-overlapping spatial regions on a single solid support (e.g. in a lateral flow assay method) to distinguish between the binding event(s) at the different serotype-specific aptamers.

In various embodiments, the method further comprises administering a DENV treatment regimen to the subject if the subject is indicated for a current DENV infection.

In various embodiments, the method further comprises a step of generating a nucleic acid library, optionally a DNA library, the nucleic acid or the DNA comprising at least one unnatural base. In various embodiments, the method further comprises a step of selecting for a candidate nucleic acid or DNA having high affinity to DENV protein, optionally to DEN-NS1 protein, by subjecting the nucleic acid to SELEX or ExSELEX, and then recovering/isolating any DENV-nucleic acid complex (or DENV-DNA complex) optionally any DEN-NS1-nucleic acid complex (or DEN-NS1-DNA complex). In some examples, an enrichment may be observed from the differences in sequence population before and after isolation of the DENV-nucleic acid complex (or DENV-DNA complex) optionally any DEN-NS1-nucleic acid complex (or DEN-NS1-DNA complex). In various embodiments, the recovering/isolating step comprises capturing the DENV-nucleic acid complex (or DENV-DNA complex), optionally the DEN-NS1-nucleic acid complex (or DEN-NS1-DNA complex), with an anti-DENV antibody, optionally an anti-DEN-NS1 antibody, or through affinity-tag (e.g. His-tag) in the protein. Any unbound nucleic acid or DNA may be washed away. The candidate nucleic acid or DNA may be isolated from the complex to obtain the aptamer, or it may be subjected to one or more round of selection by SELEX or ExSELEX. An amplification step, for example PCR amplification, may be carried out to amplify the candidate nucleic acid or DNA before the one or more round of selection or at the end of the selection. In some examples, at least about one, at least about two, at least about three, at least about four, at least about five, at least about six, at least about seven, at least about eight, at least about nine, at least about ten, at least about 11, at least about 12, at least about 13, at least about 14 or at least about 15 rounds of SELEX are carried out.

Embodiments of the method based on the detection of DENV antigen binding protein in a subject's sample may also be employed alone, independent of preceding steps of detecting for DENV protein in the sample. Even if a subject is not suffering or not suspected to be suffering from a current DENV infection, embodiments of the method which can determine whether a subject has past DENV infection(s) can be helpful. For example, the immune history may be helpful for understanding subsequent disease risk and protection, and also suitability of the subject for a DENV vaccine.

In various embodiments therefore, wherein the method is a method of identifying a past DENV infection in the subject, the contacting step is performed in the presence of a DENV protein, and an absence of a binding event at any of the aptamer(s) is indicative of a past DENV infection in the subject, optionally wherein the unbound aptamer(s) is specific to a DENV serotype and the absence of the binding event(s) is indicative of a past DENV infection of said serotype(s) in the subject.

In various embodiments therefore, there is provided method of evaluating a subject's suitability for a DENV vaccine, the method comprising: contacting a sample of the subject with an aptamer or the mixture of aptamers in the presence of a DENV protein, detecting a binding event at the aptamer(s), determining an immune history of the subject based on the binding event at the aptamer(s), and concluding the suitability of the subject for the DENV vaccine based on the immune history. In various embodiments, the absence of a binding event at any of the aptamer(s) in indicative of past DENV infection(s) in the subject.

As may be appreciated, some dengue vaccines have been shown to lead to a higher risk of more severe symptoms when a vaccinated subject subsequently becomes infected with DENV. Consequently, the vaccines are advised to be used in only subjects who were previously infected with DENV. Advantageously, embodiments of the method which can establish a subject's DENV immune history, for example whether the person has a past DENV infection, the number of past DENV infection(s), the serotypes of the past DENV infection(s), are therefore helpful in determining the person's suitability for DENV vaccination. For example, if it is determined that the subject does not have any past DENV infection, the subject may not be a suitable candidate for a DENV vaccine advised only for people with prior DENV infections.

In various embodiments, the sample comprises a biological sample. In various embodiments, the biological sample comprises a fluid biological sample or a liquid biological sample. The fluid biological sample or liquid biological sample may be blood, serum, plasma, sputum, lavage fluid, cerebrospinal fluid, urine, semen, sweat, tears, saliva, and the like. In some embodiments, the fluid biological sample or liquid biological sample comprises whole blood, blood serum, blood plasma or processed fractions thereof. In some embodiments, the fluid biological sample comprises blood serum or blood plasma. In some embodiments, the fluid biological sample comprises antigen binding proteins such as antibodies.

In various embodiments, the sample comprises a sample that is collected from a subject during an acute phase or febrile phase (about 2 days to about 7 days post illness onset (pio)), an early convalescent phase (about 10 days to about 14 days pio), a late convalescent phase (about 1 month pio), an early recovery phase (about 3 months pio), a late recovery phase (about 5 months to about 6 months pio) or a full recovery phase (about 1 year pio). In some embodiments, the sample is collected from a subject when the subject is in an early phase of DENV infection (within about 1 week pio). In some embodiments, the sample is collected from a subject when the subject is in a late phase of DENV infection (after about 1 week pio). In some embodiments, the sample is collected from a subject when subject shows symptoms associated with DENV infection, such as fever (typically high fever), headache, muscle, bone, and joint pain, nausea, vomiting, pain behind the eyes, swollen glands, rash, severe abdominal pain, persistent vomiting, bleeding from gums or nose, blood in urine, stools or vomit, bleeding under the skin, difficult or rapid breathing, cold or clammy skin (shock), fatigue, and irritability or restlessness. In some embodiments, the sample is collected from a subject when the subject is or has become asymptomatic for DENV infection. In some embodiments, the sample is collected from a subject after the subject has recovered from DENV infection.

In various embodiments, the method comprises a diagnostic method. For example, in a direct DENV protein detection method, a binding event at an aptamer may be indicative of DENV infection in the subject.

In various embodiments, the method comprises a prognosis method. In some examples (see e.g. FIG. 13), evaluation of clinical samples revealed that, in an early phase of infection (e.g. day 3-day 6 pio), DENV protein was detectable by embodiments of the direct DENV protein detection method while IgG was not detectable by embodiments of the competitive anti-DENV IgG detection method. In a latter phase of the infection (e.g. day 20 pio), however, DENV protein was no longer detected by embodiments of the direct DENV protein detection method while IgG became detectable by embodiments of the competitive anti-DENV IgG detection method. Thus, embodiments of the method may be used to monitor IgG, optionally specific IgG, production/generation in a subject, and confirm proper IgG production/generation with acquired immune system. Embodiments of the method are thus useful for prognosis. In some examples, a reduced level of binding at the aptamer in respect of a sample relative to the level of binding in respect of an earlier sample (e.g. a sample collected from the same subject at an earlier time point) may be indicative of prognosis of DENV infection in the subject.

In various embodiments, the method has high sensitivity and/or specificity. In various embodiments, the method has a sensitivity of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or at least about 100%.

In various embodiments, the method has a specificity of at least about 50%, at least about 55%, at least about 60%, at least about 65%, at least about 70%, at least about 75%, at least about 80%, at least about 81%, at least about 82%, at least about 83%, at least about 84%, at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, at least about 99% or at least about 100%.

In various embodiments, the method comprises an in vitro or an ex vivo method.

In various embodiments, there is provided a kit for identifying a DENV infection in a subject, the kit comprising the aptamer or the mixture of aptamers. In some embodiments, the aptamer or the mixture of aptamers is provided in the form of a plate coated with the aptamer or the mixture of aptamers for capturing DENV protein. In some embodiments, the kit further comprises a DENV protein, optionally a DENV-NS1 protein. In some embodiments, the kit comprises a detector agent and/or a capturing agent.

The kit may be a diagnostic kit or a prognostic kit.

In various embodiments, the subject comprises a mammal. In various embodiments, the subject comprises a human subject.

In various embodiments, there is provided a nucleic acid molecule comprising a sequence set out in Table 1, or a sequence sharing at least about 70%, at least about 75%, at least about 80%, at least about 85%, at least about 90%, at least about 91%, at least about 92%, at least about 93%, at least about 94%, at least about 95%, at least about 96%, at least about 97%, at least about 98% or at least about 99% sequence identity thereto, or a sequence differing by about one, about two, about three, about four, about five, about six, about seven, about eight, about nine, about ten, about 11, about 12, about 13, about 14, about 15, about 16, about 17, about 18, about 19, about 20, about 21, about 22, about 23, about 24 or about 25 bases or nucleotides thereto, or portions thereof, optionally linear portions thereof. In some embodiments, the aptamer comprises a sequence differing by no more than about one, no more than about two, no more than about three, no more than about four, no more than about five, no more than about six, no more than about seven, no more than about eight, no more than about nine, no more than about ten, no more than about 11, no more than about 12, no more than about 13, no more than about 14, no more than about 15, no more than about 16, no more than about 17, no more than about 18, no more than about 19, no more than about 20, no more than about 21, no more than about 22, no more than about 23, no more than about 24 or no more than about 25 bases or nucleotides with a sequence in Table 1, or portions thereof, optionally linear portions thereof.

In some embodiments, the nucleic acid molecule comprising a sequence set out in Table 1, or a sequence sharing at least 75% sequence identity thereto; or a sequence differing by one, two, three, four, five, six, seven, eight, nine or ten bases thereto; or portions thereof.

In various embodiments, there is provided a method of producing the nucleic acid molecule or the aptamer. In various embodiments, the method comprises a chemical synthesis method. In various embodiments, the method comprises an oligonucleotide synthesis method. In various embodiments, the method comprises a phosphoramidite method of oligonucleotide synthesis. The synthesis can be a solution-phase synthesis or a solid-phase synthesis. In some embodiments, the synthesis comprises a solid-phase synthesis. Other suitable methods of synthesising a nucleic acid molecule or aptamer or oligonucleotide may also be used. For example, the method may comprise a H-phosphonate method and/or a phosphotriester method of oligonucleotide synthesis.

In various embodiments, the method may comprise a step of incorporating one or more modifications to the nucleic acid molecule or the aptamer. The incorporation may be performed during and/or after synthesis of e.g. the oligonucleotide.

In various embodiments, there is provided a method, a kit or a nucleic acid molecule as described herein.

TABLE 8

| | Sequence (I= Biotin, L = Biotin-dT, x= dDs, | |
|---|---|---|
| SEQ ID NO. | Name | |
| 1 | DENVI | d = Diol1-dPa, y = Diol1-dPx, w = Diol1-dPa or Diol1-dPx) |
| | (DEN1) | DSGCVINWKGRELKCGSGIFVTNEVHTWTEQYKFQADSPKRLSAAIGKAWEEGVCGI |
| | | RSATRLENIMWKQISNELNHILLENDMKFTVVVGDVSGILAQGKKMIRPQPMEHKYS |
| | | WKSWGKAKIIGADVONTTFIIDGPNTPECPDNQRAWNIWEVEDYGFGIFTTNIWLKL |
| | | RDSYTQVCDHRLMSAAIKDSKAVHADMGYWIESEKNETWKLARASFIEVKTCIWPKS |
| | | HTLWSNGVLESEMIIPKIYGGPISQHNYRPGYFTQTAGPWHLGKLELDFDLCEGTTV |
| | | VVDEHCGNRGPSLRTTTVTGKTIHEWCCRSCTLPPLRFKGEDGCWYGMEIRPVKEKE |
| | | ENLVKSMVSA |
| 2 | DENV2 | DSGCVVSWKNKELKCGSGIFITDNVHTWTEQYKFQPESPSKLASAIQKAHEEGICGI |
| | (DEN2) | RSVTRLENLMWKQITPELNHILSENEVKLTIMTGDIKGIMQAGKRSLRPQPTELKYS |
| | | WKTWGKAKMLSTESHNQTFLIDGPETAECPNTNRAWNSLEVEDYGFGVFTTNIWLKL |
| | | KEKQDVFCDSKLMSAAIKDNRAVHADMGYWIESALNDTWKIEKASFIEVKNCHWPKS |
| | | HTLWSNGVLESEMIIPKNLAGPVSQHNYRPGYHTQITGPWHLGKLEMDFDFCDGTTV |
| | | VVTEDCGNRGPSLRTTTASGKLITEWCCRSCTLPPLRYRGEDGCWYGMEIRPLKEKE |
| | | ENLVNSLVTA |
| 3 | DENV3 | DMGCVINWKGKELKCGSGIFVTNEVHTWTEQYKFQADSPKRLATAIAGAWENGVCGI |
| | (DEN3) | RSTTRMENLLWKQIANELNYILWENNIKLTVVVGDTLGVLEQGKRTLTPQPMELKYS |
| | | WKTWGKAKIVTAETQNSSFIIDGPNTPECPSASRAWNVWEVEDYGFGVFTTNIWLKL |
| | | REVYTQLCDHRLMSAAVKDERAVHADMGYWIESQKNGSWKLEKASLIEVKTCTWPKS |
| | | HTLWTNGVLESDMIIPKSLAGPISQHNYRPGYHTQTAGPWHLGKLELDFNYCEGTTV |
| | | VITESCGTRGPSLRTTTVSGKLIHEWCCRSCTLPPLRYMGEDGCWYGMEIRPISEKE |
| | | ENMVKSLVSA |
| 4 | DENV4 | DMGCVASWSGKELKCGSGIFVVDNVHTWTEQYKFQPESPARLASAILNAHKDGVCGI |
| | (DEN4) | RSTTRLENVMWKQITNELNYVLWEGGHDLTVVAGDVKGVLTKGKRALTPPVSDLKYS |
| | | WKTWGKAKIFTPEARNSTFLIDGPDTSECPNERRAWNSLEVEDYGFGMFTTNIWMKF |
| | | REGSSEVCDHRLMSAAIKDQKAVHADMGYWIESSKNQTWQIEKASLIEVKTCLWPKT |
| | | HTLWSNGVLESQMLIPKSYAGPFSQHNYRQGYATQTVGPWHLGKLEIDFGECPGTTV |
| | | TIQEDCDHRGPSLRTTTASGKLVTQWCCRSCTMPPLRFLGEDGCWYGMEIRPLSEKE |
| | | ENMVKSQVTA |
| 5 | SINDEN1 | DSGCVINWKGRELKCGSGIFVTNEVHTWTEQYKFQADSPKRLSAAIGKAWEEGVCGI |
| | NS1 target | RSATRLENIMWKQISNELNHILLENDMKFTVVVGDANGILTQGKKMIRPQPMEHKYS |
| | (96.3) | WKSWGKAKIIGADTQNTTFIIDGPDTPECPDDQRAWNIWEVEDYGFGVFTTNIWLKL |
| | | RDSYTQMCDHRLMSAAIKDSKAVHADMGYWIESEKNETWKLARASFIEVKTCIWPRS |
| | | HTLWSNGVLESEMIIPKIYGGPISQHNYRPGYFTQTAGPWHLGKLELDFNLCEGTTV |
| | | VVDEHCGNRGPSLRTTTVTGKIIHEWCCRSCTLPPLRFRGEDGCWYGMEIRPVKEKE |
| | | ENLVRSMVSA |
| 6 | SIN DEN1 | DSGCVINWKGRELKCGSGIFVTNEVHTWTEQYKFQADSPKRLSAAIGKAWEEGVCGI |
| | NS1 target | RSATRLENIMWKQISNELNHILLENDMKFTVVVGDVAGILAQGKKMIRPQPMEHKYS |
| | (98.9) | WKSWGKAKIIGADVONTTFIIDGPNTPECPDDQRAWNIWEVEDYGFGIFTTNIWLKL |
| | | RDSYTQVCDHRLMSAAIKDSKAVHADMGYWIESEKNETWKLARASFIEVKTCIWPKS |
| | | HTLWSNGVLESEMIIPKIYGGPISQHNYRPGYFTQTAGPWHLGKLELDFDLCEGTTV |
| | | VVDEHCGNRGPSLRTTTVTGKIIHEWCCRSCTLPPLRFRGEDGCWYGMEIRPVKEKE |
| | | ENLVKSMVSA |
| 7 | DENV1 | DSGCVINWKGRELKCGSGIFVTNEVHTWTEQYKFQADSPKRLSAAIGKAWEEGVCGI |
| | (DEN1) with | RSATRLENIMWKQISNELNHILLENDMKFTVVVGDVSGILAQGKKMIRPQPMEHKYS |
| | His-tag | WKSWGKAKIIGADVQNTTFIIDGPNTPECPDNQRAWNIWEVEDYGFGIFTTNIWLKL |
| | | RDSYTQVCDHRLMSAAIKDSKAVHADMGYWIESEKNETWKLARASFIEVKTCIWPKS |
| | | HTLWSNGVLESEMIIPKIYGGPISQHNYRPGYFTQTAGPWHLGKLELDFDLCEGTTV |
| | | VVDEHCGNRGPSLRTTTVTGKTIHEWCCRSCTLPPLRFKGEDGCWYGMEIRPVKEKE |
| | | ENLVKSMVSAHHHHHH |
| 8 | DENV2 | DSGCVVSWKNKELKCGSGIFITDNVHTWTEQYKFQPESPSKLASAIQKAHEEGICGI |
| | (DEN2) with | RSVTRLENLMWKQITPELNHILSENEVKLTIMTGDIKGIMQAGKRSLRPQPTELKYS |
| | His-tag | WKTWGKAKMLSTESHNQTFLIDGPETAECPNTNRAWNSLEVEDYGFGVFTTNIWLKL |
| | | KEKQDVFCDSKLMSAAIKDNRAVHADMGYWIESALNDTWKIEKASFIEVKNCHWPKS |
| | | HTLWSNGVLESEMIIPKNLAGPVSQHNYRPGYHTQITGPWHLGKLEMDFDFCDGTTV |
| | | VVTEDCGNRGPSLRTTTASGKLITEWCCRSCTLPPLRYRGEDGCWYGMEIRPLKEKE |
| | | ENLVNSLVTAHHHHHH |
| 9 | DENV3 | DMGCVINWKGKELKCGSGIFVTNEVHTWTEQYKFQADSPKRLATAIAGAWENGVCGI |
| | (DEN3) with | RSTTRMENLLWKQIANELNYILWENNIKLTVVVGDTLGVLEQGKRTLTPQPMELKYS |
| | His-tag | WKTWGKAKIVTAETQNSSFIIDGPNTPECPSASRAWNVWEVEDYGFGVFTTNIWLKL |
| | | REVYTQLCDHRLMSAAVKDERAVHADMGYWIESQKNGSWKLEKASLIEVKTCTWPKS |
| | | HTLWTNGVLESDMIIPKSLAGPISQHNYRPGYHTQTAGPWHLGKLELDFNYCEGTTV |
| | | VITESCGTRGPSLRTTTVSGKLIHEWCCRSCTLPPLRYMGEDGCWYGMEIRPISEKE |
| | | ENMVKSLVSAHHHHHH |
| 10 | DENV4 | DMGCVASWSGKELKCGSGIFVVDNVHTWTEQYKFQPESPARLASAILNAHKDGVCGI |
| | (DEN4) with | RSTTRLENVMWKQITNELNYVLWEGGHDLTVVAGDVKGVLTKGKRALTPPVSDLKYS |
| | His-tag | WKTWGKAKIFTPEARNSTFLIDGPDTSECPNERRAWNSLEVEDYGFGMFTTNIWMKF |
| | | REGSSEVCDHRLMSAAIKDQKAVHADMGYWIESSKNQTWQIEKASLIEVKTCLWPKT |
| | | HTLWSNGVLESQMLIPKSYAGPFSQHNYRQGYATQTVGPWHLGKLEIDFGECPGTTV |
| | | TIQEDCDHRGPSLRTTTASGKLVTQWCCRSCTMPPLRFLGEDGCWYGMEIRPLSEKE |
| | | ENMVKSQVTAHHHHHHHHHH |
| 11 | D11-48h | CCCCAGACGGACTGGTGTxCTCGGxATGGCCGTCTGGGGCGCGLAGCG |
| 12 | D2-1d-72h | GGCTGGTCCGxCTGGGAACAAGxGGCGGGAGGGAdGGGTGTGGGTGCGACAAGCGGA |
| | | CCAGCCCGCGLAGCG |
| 13 | D3-2-59h | CCGCTTGTCATCTAxCCTGGCCxTGTGGTACTGTAACGGCTGACAAGCGGCGCGLAG |
| | | CG |
| 14 | D4-3-57h | CGGCGGAGACGTAACGCxTATCAAATCxAAACAGCTTAGGGTCCGCCGCGCGLAGCG |
| 15 | 19D1F1 | Biotin-TTTCGCACTCCATGATATGGTCTACTGAGCGAGACGATGCTGCTAAAxTA |
| | (isolate) | CGCCGTGGTxACGAAGACAGACAAGCGGAGTAGTTAGACCGTGAAA |
| 16 | 19D1F1-3 | GCACTCCATGATATGGTCTACTGAGCGAGACGATGCTGCTAAAxTACGCCGTGGTxA |
| | | CGAAGACAGACAAGCGGAGTGTCGCGLAGCG |
| 17 | D1-1-78 | LGATATGGTCTACTGTGTGAxGTCCTACAATGGACTGGTGTxCTCGGxATGGCCATT |
| | | GACAAGCGGAGTAGTTAGACC |
| 18 | D1-1-42h | CAGACGGACTGGTGTxCTCGGxATGGCCGTCTGCGCGLAGCG |
| 19 | D2-1d-97 | LTTTCGCACTCCATGATATGGTCTACTGGTCCGxCTGGGAACAAGxGGCGGGAGGGA |
| | | dGGGTGTGGGTGCGACAAGCGGAGTAGTTAGACCGTCAAA |
| 20 | D2-1y-96 | Biotin-TTTCGCACTCCATGATATGGTCTACTGGTCCGxCTGGGAACAAGxGGCGG |
| | | GAGGGAyGGGTGTGGGTGCGACAAGCGGAGTAGTTAGACCGTCAAA |
| 21 | D2-1d-84 | LTTTCGCACTCCATGATATGGTCTACTGGTCCGxCTGGGAACAAGxGGCGGGAGGGA |
| | | dGGGTGTGGGTGCGACAAGCGGAGTAG |
| 22 | D2-1d-74 | LCATGATATGGTCTACTGGTCCGxCTGGGAACAAGxGGCGGGAGGGAdGGGTGTGGG |
| | | TGCGACAAGCGGAGTAG |
| 23 | D2-1d-87h | GACGGTCTACTGGTCCGxCTGGGAACAAGxGGCGGGAGGGAdGGGTGTGGGTGCGAC |
| | | AAGCGGAGTAGTTAGACCGTCCGCGLAGCG |
| 24 | D2-1d-77h | GGTCTACTGGTCCGxCTGGGAACAAGxGGCGGGAGGGAdGGGTGTGGGTGCGACAAG |
| | | CGGAGTAGACCCGCGLAGCG |
| 25 | D2-1d-61h | GGTCCGxCTGGGAACAAGxGGCGGGAGGGAdGGGTGTGGGTGCGACAAGCGGCGCGL |
| | | AGCG |
| 26 | D3-2-78 | LGATATGGTCTACTGAAGTGTTGTCATCTAxCCTGGCCxTGTGGTACTGTAACGGCT |
| | | GACAAGCGGAGTAGTTAGACC |
| 27 | D4-3-78 | LGATATGGTCTACTGTGGCGCGAGGGAATCxACGCxTATCAAATAxAAACAGCTAAT |
| | | GACAAGCGGAGTAGTTAGACC |
| 28 | D1-2-78 | LGATATGGTCTACTGAGGAGCGCATGTCGAGATACCAACCxCCATCCAATCxTTCTT |
| | | GACAAGCGGAGTAGTTAGACC |
| 29 | D1-3-78 | LTGATATGGTCTACTGACGCCGGGGCCCGTAxTCAGACGTATACxCATCAGGGCACA |
| | | TACAAGCGGAGTAGTTAGACC |
| 30 | D1-3-47 | CGAGGCCCGTAxTCAGACGTATACxCATCAGGGCCTCGCGCGLAGCG |
| 31 | D1-5-61h | GGCAGCGCGTCGATTGxCCAATCTTAGCCAACCCAAAATTACAAGCGCTGCCCGCGL |
| | | AGCG |
| 32 | D1-6-47h | GCTGCCTxGTACCAACCCCCTCCAATCxATTAGGCAGCCGCGLAGCG |
| 33 | D1-7-51h | CGTGCGACGAxGTCCAACCAGTCCCAATCxACAAGTCGCACGCGCGLAGCG |
| 34 | D2-2d-59h | GCGGTCCGTGCxGTCGCCAATCCGTGdTCCAACCCCGACAAGCGGACCGCCGCGLAG |
| | | CG |
| 35 | D2-3d-52h | GCCCGCTTTCGxCCAACCCGTGdTCCAATCCCAGAAAGCGGGCCGCGLAGCG |
| 36 | D2-4d-56h | CGCCCGTCAAGGxCTCCAATCCGTGdTCCAACCAGTTTTGACGGGCGCGCGLAGCG |
| 37 | D2-5-46h | GCCCGCGTGCTCAACCTTACCAATCTGxCACGCGGGCCGCGLAGCG |
| 38 | D2-5-48h | GCCCTGCGxGCTCAACCTTACCAATCTGxCACGCAGGGCCGCGLAGCG |
| 39 | D3-1-85 | LACTCCATGATATGGTCTACTGATAGTACTCCxGTTTAACTCTGAxACTTGACGTCC |
| | | ATTCATAGACAAGCGGAGTAGTTAGACC |
| 40 | D3-3-78 | LGATATGGTCTACTGGGGCTTGGTCTTGCGTxTGCAGATTAACTTGCGTGCCAGTAA |
| | | GACAAGCGGAGTAGTTAGACC |
| 41 | D4-1-78 | LGATATGGTCTACTGTCTCAACGGTTGTCAAACGGxTATCACGGCxACACACCTGCG |
| | | GACAAGCGGAGTAGTTAGACC |
| 42 | D4-1-57h | CTCCGCTGTCAAACGGxTATCACGGCxACACACCTGCGGACAGCGGAGCGCGLAGCG |
| 43 | D4-2-78 | LGATATGGTCTACTGTCACAxATCGCCGTAAAGxCGAAGAGCTGCGGAATCTAAGGT |
| | | GACAAGCGGAGTAGTTAGACC |
| 44 | D4-4-78 | LGATATGGTCTACTGTATAATCCGCxTTCGTCATGTGGxTTGGATCTGGGTCTGGCA |
| | | GACAAGCGGAGTAGTTAGACC |
| 45 | D4-5-78 | LGATATGGTCTACTGCCCAAxCTTGTCTGTAAGGGxTTGGxTAGGGCTGGCAAAAAA |
| | | GACAAGCGGAGTAGTTAGACC |
| 46 | 19D1F1-1 | CGGCCGATGCTGCTAAAxTACGCCGTGGTxACGAAGACAGACAAGCGGAGTAGTTAG |
| | | ACCGGCCGCGCGLAGCG |
| 47 | 19D1F1-2 | GCGCCAAAxTACGCCGTGGTxCGAAGACAGACAAGCGGAGTAGTTGGCGCCGCGLAG |
| | | CG |
| 48 | 19D1F1-4 | GCACTCCGTCTACTGAGCGAGACGATGCTGCTAAAxTACGCCGTGGTxACGAAGACG |
| | | GAGTGTCGCGLAGCG |
| 49 | 19D1F1-5 | GCACTCCGCTACTGAGCGAGACGATGCTGCTAAAxTACGCCGTGGTxACGAAGACAG |
| | | CGGAGTGTCGCGLAGCG |
| 50 | D2-1d-72h | GGCTGGTCCGACTGGGAACAAGxGGCGGGAGGGAdGGGTGTGGGTGCGACAAGCGGA |
| | (A) | CCAGCCCGCGLAGCG |
| 51 | D1-1-48h/ | ACTGGTGTxCTCGGxATGG |
| | D1-1-78/ | |
| | D1-1-42h | |
| | (core) | |
| 52 | D2-1d-72h/ | TGGGAACAAGxGGCGGGAGGGAwGGGTGTGGGTGCGACAAG |
| | D2-1d-97/ | |
| | D2-1d-84/ | |
| | D2-1d-74/ | |
| | D2-1d-87h/ | |
| | D2-1d-77h/ | |
| | D2-1d-61h/ | |
| | D2-1y-96 | |
| | (core) | |
| 53 | D3-2-59h/ | TCTAxCCTGGCCxTGTGGTACTGTAACGGC |
| | D3-2-78 | |
| | (core) | |
| 54 | D4-3-57h | GACGTAACGCxTATCAAATCxAAACAGCT |
| | (core) | |
| 55 | 19D1F1 | ATGATATGGTCTACTGAGCGAGACGATGCTGCTAAAxTACGCCGTGGTxACGAAGAC |
| | (isolate)/ | AGACAAGC |
| | 19D1F1-3 | |
| | (core) | |
| 56 | D4-3-78 | GAGGGAATCxACGCxTATCAAATAxAAACAGCT |
| | (core) | |
| 57 | D4-1-78/ | AAACGGxTATCACGGCxACACACCTGCG |
| | D4-1-57h | |
| | (core) | |

### BRIEF DESCRIPTION OF FIGURES

FIG. 1. ExSELEX scheme to generate UB-DNA aptamers targeting each DEN-NS1 serotype using an aptamer-antibody sandwich method. The Ds-containing DNA library was mixed with each target DEN-NS1. The DNA—protein complexes were captured with the immobilized anti-NS1 antibody. After washing to remove the unbound DNA species, the bound DNA species are recovered and subjected to PCR amplification involving the Ds-Px pair as a third base pair, to obtain the enriched DNA library for the next round of selection. After several ExSELEX rounds, the sequences in the enriched DNA libraries were determined by deep sequencing, and the aptamer candidates were optimized and stabilized by adding a mini-hairpin DNA. It was found that one of the aptamer candidates contained the Px base, which resulted from the mutation from the natural base to Px during PCR amplification. Since the Px nucleoside is unstable during DNA chemical synthesis, the aptamers were synthesized using the Pa(diol) nucleotide, instead of Px(diol).

FIG. 2. Binding analysis of DNA libraries by electrophoresis gel-mobility shift assays The enriched DNA libraries (50 nM) in the final round of three independent ExSELEX procedures (ExSELEX-1, ExSELEX-2, and ExSELEX-3 targeting each DEN-NS1 protein) were incubated with DEN1-NS1, DEN2-NS1, DEN3-NS1 or DEN4NS1 (25 nM, as hexamers) at 25° C. for 30 min, and the DNA-NS1 complexes were separated from the free DNA on native 4% acrylamide gels. The DNA band patterns on the gels were detected with a bio-imaging analyzer, after staining the DNA bands with SYBR Gold. To investigate the importance of the Ds bases in the DNA libraries, DNA libraries without the Ds bases were prepared the by replacement PCR, and compared the binding patterns (Ds vs. Ds→natural base (NB)). In all cases, the densities of the shifted bands corresponding to the respective complexes were reduced in the absence of the Ds bases, suggesting that the binding species are dependent on the Ds bases for their target binding.

FIG. 3. Alignment of the random-region DNA sequences obtained by the three ExSELEX procedures, targeting DEN1-NS1. The sequences were obtained by deep sequencing through replacement PCR, by using intermediate unnatural-base substrates. The unnatural-base positions, indicated by “x”, were predicted from the mutation spectra (natural-base composition rates) after replacement PCR. The ratio (%) of each family was calculated from the total counts categorized in the same family against the total extracted reads for the analysis. Several representative family sequences, from D1-1 to D1-7, were chosen for binding analyses by EMSA and SPR (summarized in Table E2).

FIG. 4. Alignment of the random-region DNA sequences obtained by the three ExSELEX procedures, targeting DEN2-NS1. The sequences were obtained by deep sequencing through replacement PCR, by using intermediate unnatural-base substrates. The unnatural-base positions, shown as “x”, were predicted from the mutation spectra (natural-base composition rates) after replacement PCR. The ratio (%) of each family was calculated from the total counts categorized in the same family against the total extracted reads for the analysis. Several representative family sequences, from D2-1 to D2-6, were chosen for binding analyses by EMSA and SPR (summarized in Table E2).

FIG. 5. Alignment of the random-region DNA sequences obtained by the three ExSELEX procedures, targeting DEN3-NS1. The sequences were obtained by deep sequencing through replacement PCR, by using intermediate unnatural-base substrates. The unnatural-base positions, indicated by “x”, were predicted from the mutation spectra (natural-base composition rates) after replacement PCR. The ratio (%) of each family was calculated from the total counts categorized in the same family against the total extracted reads for the analysis. Several representative family sequences, from D3-1 to D3-3, were chosen for binding analyses by EMSA and SPR (summarized in Table E2).

FIG. 6. Alignment of the random-region DNA sequences obtained by the three ExSELEX procedures, targeting DEN4-NS1. The sequences were obtained by deep sequencing through replacement PCR, by using intermediate unnatural-base substrates. The unnatural-base positions, indicated by “x”, were predicted from the mutation spectra (natural-base composition rates) after replacement PCR. The ratio (%) of each family was calculated from the total counts categorized in the same family against the total extracted reads for the analysis. Several representative family sequences, from D4-1 to D4-5, were chosen for binding analyses by EMSA and SPR (summarized in Table E2).

FIG. 7. Presumed secondary structures of UB-DNA aptamers that bind specifically to each DEN-NS1 serotype and serotype-specific DEN-NS1 detection by ELISA in combination with the UB-DNA aptamer and antibody (Ab#D06) pair. a, Each aptamer specifically bound to each DENNS1 serotype: AptD1 (D1-1-48 h) to DEN1-NS1, AptD2 (D2-1d-72 h) to DEN2-NS1, AptD3 (D3-2-59 h) to DEN3-NS1, and AptD4 (D4-3-57 h) to DEN4-NS1. Each aptamer's kinetic binding parameters, dissociation constant (K_{D}), and association and dissociation rates (kₒₙ and k_{off}) were determined by SPR analysis (FIG. 10). All aptamers contain two Ds bases, while AptD2 contains one Pa and two Ds bases, which are essential for tight binding to the target. The Ds and Pa bases are indicated in bigger circles, compared with those of the natural bases. AptD2 has several G-motifs, shown in bold, in a large loop region. The mini-hairpin DNA sequences, CGCGTAGCG, are attached to the 3′-terminus. The thymidines within the mini-hairpin DNA sequences are used as the biotinylation sites. b, Each UB-DNA aptamer specifically recognized the targeted DEN-NS1, allowing for specific NS1 detection. In ELISA, a 10-μl portion of a 100 ng/ml solution of each flavivirus NS1 protein (DENV serotype 1-4 NS1 proteins, and Zika virus NS1 proteins of a Brazilian strain and a Ugandan strain) was used in buffer. The sample size is two per each combination set, and the error bars represent one standard deviation. The bars with wavy lines indicate that at least one of the two sample wells showed overflow (OD₄₅₀>4.000).

FIG. 8. Confirmation of the presence of diol-Px in the selected clone family, D2-1. (A) Scheme of the series of experiments. First, the D2-1 clones were isolated using a biotinylated specific probe from the enriched library of Round 7 in ExSLEX-3 targeting DEN2-NS1, and were amplified by 20-cycle PCR in the presence of unnatural substrates, dDsTP and diol-dPxTP. By denaturing PAGE, the aptamer strand was purified and its binding to the target was examined by EMSA, as shown in panel B. The aptamer strand was further amplified by PCR in the presence of dDsTP and Cy5-dPxTP, using a FAM-labeled primer, for the specific labeling of the aptamer strand at the 5′-end. To assess the presence of Px in the aptamer strand from the Cy5-Px incorporation, the PCR products of the aptamer strand were analyzed by denaturing PAGE and the product patterns were compared with those from the PCR product of the initial Ds-DNA library. The aptamer strand and FAM-labeled primer were detected by the FAM fluorescence and the presence of Px was detected by the Cy5 fluorescence (panel C). After purification of the FAM-labeled aptamer strand and the Ds-library strand, they were treated with a concentrated ammonia solution at 55° C. for 4 hours to cleave the DNA fragment at the Px position. After the treatment, the DNA band patterns were analyzed by denaturing PAGE. (B) Gel mobility shift patterns support the binding of the aptamer strand to the target DEN2 NS1, but the D2-1-96(3Ds), in which the predicted unnatural base positions are all Ds (see Table E2), did not bind to the target. (C) When the initial Ds-DNA library was used as the template for PCR, the band corresponding to the aptamer strand was detected only by FAM fluorescence while the band corresponding to the complementary strand was detected only by Cy5 fluorescence. However, when the isolated clone was used as the template for PCR, the band corresponding to the amplified aptamer strand was detected by FAM and Cy5 fluorescnes. The DNA band patterns on the gel indicate that the aptamer strand that was PCR-amplified from the isolated clone should contain a Px base, since PCR under the same conditions using an initial Ds-DNA library only produced the aptamer strand without Px bases and the complementary strand with Px bases. (D) The DNA band patterns on the gel indicate that the aptamer strand was cleaved at a specific position, probably due to the presence of the Px base, not the Ds base, at the specific position corresponding to the predicted, third Ds base.

FIG. 9. Electrophoresis gel-mobility shift assay (EMSA) of the aptamer—NS1 complex formation using anti-dengue-NS1 aptamers and their variants without unnatural bases. The DNA sequences used in the assay are listed in Table E2. DNA (50 nM) was incubated with 25 nM of the respective NS1 proteins (DEN1, DEN2, DEN3, DEN4, Zika Brazil strain (B), and Zika Uganda strain (U)) at 25° C. for 30 min, and the complexes were separated on 4% acrylamide gels. The DNA bands on the gels were stained with SYBR Gold and detected by a bio-imaging analyzer.

FIG. 10. Binding analysis of UB-DNA aptamers, D1-1-48 h, D2-1d-72 h, D3-2-59 h, and D4-3-57 h, to each target by a Biacore T200 SPR system at 25° C. Running buffer: 20 mM Tris-HCl (pH 7.5), 150 mM NaCl, 1 mM MgCl₂, 2.7 mM KCl, and 0.05% Tween 20. Flow rate: 30 μl/min. Injection (association) time: 150 sec. Dissociation time: 600 sec (general) or 1,200 sec for determination of kinetic parameters. The kinetic parameters, association rates (kₒₙ), dissociation rates (k_{off}), and dissociation constants (K_{D}), were determined through 1:1 global curve fitting with the BIAevaluation software version 3.0, by using the double-reference subtraction method. Representative association and dissociation curves (thick lines) with fitting (thin lines) are shown. Regeneration was performed with a 5-sec injection of 50 mM NaOH, followed by a 10-min equilibration with running buffer.

FIG. 11: Limit of detection (LOD) and limit of quantification (LOQ) targeting each dengue serotype NS1 by a sandwich-type ELISA using UB-DNA aptamers as capture agents and an anti-DEN-NS1 monoclonal antibody (Ab#D06) as the primary detector agent. For the target binding process, 10 μl of serially diluted NS1 (0 to 100 ng/ml) was used in a buffer with and without human serum (10%). The sample size is two per each combination set, and the data are arranged from two independent experiments. The error bars represent one standard deviation. The bars with wavy lines indicate that at least one of the two sample wells showed overflow (OD₄₅₀>4.000).

FIG. 12. Development of two ELISA formats for direct NS1 detection and IgG detection in patient blood samples. a, Schematic illustration of the general detection patterns of DENV, DENNS1, and DEN-reactive IgG and IgM in primary infection. b, Direct DEN-NS1 detection by ELISA using each UB-DNA aptamer as the capture agent and the anti-DEN-NS1 antibody (Ab#D06) as the primary detector agent. c, IgG antibody detection by a competitive ELISA format. The IgG antibodies to DEN-NS1 in the patient's serum inhibited the direct DEN-NS1 detection. Using the inhibition, the IgG detection method was developed by the competitive ELISA format by adding the authentic DENNS1 of each serotype. From the inhibition data, a simple quantification method for the IgG activities to each DEN-NS1 serotype was developed (FIG. 16).

FIG. 13. DEN-NS1 and IgG detection in eleven Singaporean clinical samples by the ELISA formats. The serotype of the current infection for each sample was determined by RT-PCR and DNA sequencing. The serotype of the past infection was estimated by the Anti-NS1 IgG detection method (the competitive ELISA). DEN-NS1, IgM, and IgG were also detected by using Alere's LFA (SD BIOLINE Dengue NS1 Ag rapid test (SD) for DEN-NS1 and Panbio Dengue Duo Cassette for IgM and IgG (Panbio)). The discrepancy of the results between by the competitive ELISA and Panbio Dengue Duo Cassette is found in PD1-1, PD2-1, PD3-2, and PD3-3. The amino-acid sequence homologies were determined from the sequence data (FIG. 14). The DEN-NS1 detection was performed by the ELISA formats, using aptamer-antibody (Ab#D06) and antibody (Ab#D25)-antibody (Ab#D06) pairs. Thick arrows in the NS1 direct detection column indicate the detectable DEN-NS1 serotypes. The DEN-NS1 proteins of the PD1-2, PD1-3, PD2-2, and PD2-3 samples were not detected by the aptamer—Ab#D06 ELISA format, because the homologies of these Singaporean DEN-NS1 proteins were lower than 96.9% of the initial target of DEN-NS1. The DEN-NS1 of PD3-4 was not detected by both the aptamer—Ab#D06 and Ab#D25—Ab#D06 ELISA formats, because of the inhibition by the IgG antibodies. The IgG detection was performed by the competitive ELISA format, using the longitudinal serum samples.

FIG. 14. Differences in the amino acid sequences of DEN-NS1 proteins in the clinical samples. (A) Alignment of the amino acid sequences of DEN-NS1 proteins in clinical samples and each recombinant DENV NS1 protein used in aptamer generation as the target. The common amino acids in the sequences are abbreviated as asterisks. Each serotype categorization is: DEN1-NS1 [D1 target (The Native Antigen Company), PD1-1 and PD1-2/1-3], DEN2-NS1 [D2 target (The Native Antigen Company), PD2-1 and PD2-2/2-3], DEN3-NS1 [D3 target (The Native Antigen Company), PD3-1, PD3-2, PD3-3, and PD3-4], and DEN4-NS1 [D4 target (The Native Antigen Company) and PD4-1]. Amino acids that are different from those in each targeted serotype NS1 protein are highlighted in light grey. (B) Summary of the homology (sequence identity) of the NS1 sequences, with mutation numbers, compared with each target NS1 protein sequence. The samples, in which NS1 was successfully detected with the ELISA format, using the specific UB-DNA aptamers, are highlighted in light grey.

FIG. 15. Inhibitory effects of human sera against direct NS1 detection. Inhibitory effects of different human serum samples were analyzed by ELISA, using the aptamer—Ab#D06 pair. Each UBDNA aptamer was used as the capture agent, and Ab#D06 was used as the primary detector agent. Each NS1 protein serotype was added to buffer (a), human serum purchased from Sigma (untreated (b) or treated with protein A resin for IgG removal (c)), or human sera obtained from different people (d, e, and f). The solutions were subjected to ELISA (final 10% human serum concentration). The amount of recombinant NS1 protein added to each well (50 μl) was 350 pg for DEN1-NS1, 350 pg for DEN2-NS1, 450 pg for DEN3-NS1, and 200 pg for DEN4-NS1.

FIG. 16. Quantification of relative anti-DEN-NS1 IgG activities in competitive IgG detection with the competitive ELISA format. The scheme to quantify the relative anti-DEN-NS1 IgG activity in human serum, based on the results of competitive IgG detection with ELISA, is illustrated by using the PD2-2 sample, obtained 9 days after the onset of fever, as an example.

FIG. 17. Comparison of sensitivities of competitive IgG detection by two different ELISA formats, Apt/Ab and Ab/Ab pairs. Inhibitory effects of each clinical human serum sample were analyzed with two ELISA formats. One uses Apt/Ab pairs, where the amount of recombinant NS1 protein added in each well is 350 pg for DEN1-NS1, 350 pg for DEN2-NS1, 450 pg for DEN3-NS1, and 200 pg for DEN4-NS1. The other uses the Ab/Ab pair (biotinylated Ab#D06 as the primary detector agent and Ab#D25 as the capture agent), where the amount of recombinant NS1 protein added in each well is 400 pg (DEN1-NS1), 250 pg (DEN2-NS1), 400 pg (DEN3-NS1), and 300 pg (DEN4NS1).

FIG. 18. Comparison of sensitivities of competitive IgG detection by two different ELISA formats, Apt/Ab and Ab/Ab pairs. Inhibitory effects of each clinical human serum sample were analyzed with the two ELISA formats. One uses Apt/Ab pairs, where the amount of recombinant NS1 protein added in each well is 350 pg for DEN1-NS1, 350 pg for DEN2-NS1, 450 pg for DEN3-NS1, and 200 pg for DEN4-NS1. The other uses the Ab/Ab pair (biotinylated Ab#D06 as the primary detector agent and Ab#D25 as the capture agent), where the amount of recombinant NS1 protein added in each well is 400 pg (DEN1-NS1), 250 pg (DEN2-NS1), 400 pg (DEN3-NS1), and 300 pg (DEN4-NS1).

FIG. 19. (A) NS1 sequence variations of dengue serotype 1 and 2 patient samples. (B) The amino acids that differed from those in each target dengue NS1 protein from the Native Antigen Company were mapped onto the tertiary structure of the dengue NS1 dimer (PDB: 4O6B). The amino acid variations found in PD1-1 and PD2-1 are indicated in grey, while those in PD1-2/PD1-3 and PD2-2/PD2-3, which might include critical amino acids for the aptamer binding, are indicated in bold with underlined.

FIG. 20. DEN-NS1 direct detection in five Singaporean clinical samples by the ELISA formats. For each sample, the serotype of the current infection was determined by RT-PCR and DNA sequencing. DEN-NS1, IgM, and IgG were detected by using Alere's LFA (SD BIOLINE Dengue NS1 Ag rapid test (SD) for DEN-NS1 and Panbio Dengue Duo Cassette for IgM and IgG (Panbio)). The amino-acid sequence homologies were determined from the sequence data in FIG. 21. The DEN-NS1 detection was performed by using the ELISA formats, with aptamer-antibody (Ab#D06) and antibody (Ab#D25)-antibody (Ab#D06) pairs. The DEN1-NS1 proteins of the PD1-2, PD1-3, and PD1-4 samples were not detected by the aptamer—Ab#D06 ELISA format, because the homologies of these Singaporean DEN1-NS1 proteins were lower than 96.9% of the initial target of DEN1-NS1, purchased from Native Antigen Company. The DEN1-NS1 of PD1-5 was detected less robustly by both the aptamer—Ab#D06 and Ab#D25—Ab#D06 ELISA formats, probably due to the low NS1 level.

FIG. 21. Comparison of the amino acid sequences of DEN1-NS1 proteins in the clinical samples. (A) Alignment of the amino acid sequences of dengue NS1 proteins in the clinical samples (PD1-1, PD1-2, PD1-3, PD1-4, and PD1-5) and the DEN1-NS1 protein used in the aptamer AptD1 generation as the target. NA DEN1-NS1: DEN1-NS1 purchased from Native Antigen Company. The amino acids that are identical to those in NA DEN1-NS1 are indicated in a grey background. (B) Summary of homology (sequence identity) of the NS1 sequences. (C) Purity check of NA_D1 and the prepared Singaporean DEN1-NS1 recombinant protein (SIN DEN1-NS1) by SDS-PAGE. The protein bands were detected by sliver staining (Bio-Rad Laboratories).

FIG. 22. Binding analysis of DNA libraries and isolated clones by gel-mobility shift assays. A 50 nM portion of the DNA library (A) or the isolated clone (B) in the final round of ExSELEX-4 was incubated with 25 nM (as hexamer) of Singaporean DEN1-NS1 (SIN DEN1-NS1), as well as DEN1-NS1, NA DEN2-NS1, NA DEN3-NS1 or NA DEN4-NS1, purchased from Native Antigen Company, at 25° C. for 30 min. The DNA-NS1complexes were separated from the free DNAs on native 4% acrylamide gels. The DNA band patterns on the gels were detected with a bio-imaging analyzer (LAS-4000, Fuji Film), after staining the DNA bands with SYBR Gold. The enriched library and the isolated clone specifically bound to Singaporean DEN1-NS1 (SIN DEN1-NS1). To investigate the importance of the Ds bases in the isolated clone, DNA without the Ds bases was prepared by replacement PCR, and compared the binding patterns (Ds vs. Ds→Natural Base). The densities of the shifted bands corresponding to the complexes were reduced in the absence of the Ds bases, suggesting that the binding species are dependent on the Ds bases for their target interactions.

FIG. 23. Alignment of the random-region DNA sequences obtained by the ExSELEX procedures, targeting SIN DEN1-NS1. (A) The sequences were obtained by deep sequencing, through replacement PCR using intermediate unnatural-base substrates. The unnatural-base positions, indicated by “X”, were predicted from the mutation spectra (natural-base compositions rates) after replacement PCR. The ratio (%) of Family 1 was calculated against the total read counts categorized in the same family against the total extracted reads (43,385) for the analysis. (B) Summary of oligonucleotide sequences used for 19D1F1 characterization in the ELISA and SPR analysis. The dissociation constants determined by SPR and the colorimetric absorbance data in ELISA are included. N.D.: not determined (too weak to calculate the dissociation constant). N.A.: not assayed. The oligonucleotides containing a mini-hairpin sequence, CGCG-(Biotin-T)-AGCG, at the 3′-terminus are underlined. The nucleotides that did not originate from the 19D1F1 sequence are shown in light grey.

FIG. 24. Binding analysis of UB-DNA aptamers, 19D1F1-3 and 19D1F1 (isolate) to Singaporean DEN1-NS1 and NA DEN1-NS1 by a Biacore T200 SPR system at 25° C. Running buffer: 20 mM Tris-HCl (pH 7.5), 150 mM NaCl, 1 mM MgCl₂, 2.7 mM KCl, and 0.05% Tween20. Flow rate: 30 μl/min. Injection (association) time: 150 sec. Dissociation time: 600 sec (general) or 1,200 sec for determination of kinetic parameters. The kinetic parameters, association rates (kₒₙ), dissociation rates (k_{off}), and dissociation constants (K_{D}), were determined through 1:1 global curve fitting with the BIAevaluation software version 3.0, by using the double-reference subtraction method. Representative association and dissociation curves (thick lines) with fitting (thin lines) are shown. Regeneration was performed with a 5-sec injection of 50 mM NaOH, followed by a 10-min equilibration with running buffer. (A) Singaporean DEN1-NS1 and (B) NA DEN1-NS1 recombinant protein as the analytes were used for the K_{D} measurements.

FIG. 25. Comparison of ELISA signal patterns using AptD1 (D1-1-48 h) and AptD1b (19D1F1 isolate) in direct NS1 detection. For the detection samples, different clinical serums (PD1-1, PD1-2, and PD1-3) and DEN-NS1 recombinant proteins from Native Antigen Company (D1 NA, D2 NA, D3 NA, and D4 NA) were used. The DEN-NS1 detection was performed by the ELISA formats, using the aptamer-antibody (Ab#D06) pair. The DEN1-NS1 proteins of the PD1-1 sample and D1 NA were detected by the AptD1-Ab#D06 ELISA format, while the DEN1-NS1 proteins of the PD1-2 and PD1-3 samples were detected only by the AptD1b-Ab#D06 ELISA format. In the top panel, the mutated amino acid positions are indicated in grey with or without circles. The circled residues are possibly involved in the aptamer recognition specificity.

FIG. 26. Characterisation of a protected diol-Pa phosphoramidite for chemical DNA synthesis in accordance with embodiments of the invention. The chart is ¹H NMR spectrum (400 MHz. DMSO-d₆) of 1-(5-O-DMTr-2-deoxy-β-D-ribofuranosyl)-(S)-4-(4,5-dibenzoyloxy-pent-1-yn-1-yl)-1H-pyrrole-2-carbaldehyde phosphoramidite. The chemical structure of the compound is shown on the top on the left.

FIG. 27. Characterisation of a protected diol-Pa phosphoramidite for chemical DNA synthesis in accordance with embodiments of the invention. The chart is ³¹ P NMR spectrum (162 MHz, DMSO-d₆) of 1-(5-O-DMTr-2-deoxy-β-D-ribofuranosyl)-(S)-4-(4,5-dibenzoyloxy-pent-1-yn-1-yl)-1H-pyrrole-2-carbaldehyde phosphoramidite.

FIG. 28. Replacement of the unnatural bases in aptamer D2-1d-72 h. (A) Aptamers D2-1d-72 h-b, D2-1d-72 h-c and D2-1d-72 h-d were generated with a Ds→A replacement at the 11th position (D2-1d-72 h-b), a Ds→A replacement at the 23^{rd} position (D2-1d-72 h-c) and a Diol-Pa→T replacement at the 35th position (D2-1d-72 h-d) respectively. (B) Aptamer D2-1d-72 h-b retained its binding affinity to DEN2-NS1, whereas the binding affinity was abolished in aptamers D2-1d-72-c and D2-1d-72 h-d. (C) Schematic diagram of aptamer D2-1d-72 h. The Ds base at position 2803 and the diol-Pa/diol-Px base at position 2805 are shown to contribute to the aptamer's binding affinity for DEN2-NS1. Base variation at position 2801 may be tolerated.

### EXAMPLES

Example embodiments of the disclosure will be better understood and readily apparent to one of ordinary skill in the art from the following discussions and if applicable, in conjunction with the figures. It should be appreciated that other modifications related to structural, electrical and optical changes may be made without deviating from the scope of the invention. Example embodiments are not necessarily mutually exclusive as some may be combined with one or more embodiments to form new exemplary embodiments.

### UB-DNA Aptamer Generation Targeting Each DEN-NS1 Serotype

To generate Ds-containing DNA aptamers targeting each DEN-NS1 serotype, the ExSELEX procedure was performed three times (Table E1)^{33-35,38}.

TABLE E1
ExSELEX conditions targeting each DEN-NS1 serotype.
ExSELEX-1

| | | DNA | Target | Volume | | Binding | | | Counter | PCR cycles | | | |
|---|---|---|---|---|---|---|---|---|---|---|---|---|---|
| Round | Method | [nM] | [nM] | [mL] | Additives | Buffer | Time (min) | Washing | Selection | D1 | D2 | D3 | D4 |
| 1 | A | 500 | 5 | 8 | — | BB1 | 60 | BB1 × 3 | — | 18 | 18 | 18 | 18 |
| 2 | A | 100 | 5 | 1 | — | BB1 | 30 | BB1 × 5 | Pre | 10 | 12 | 10 | 12 |
| 3 | B | 20 | 4 | 0.2 | 0.1% BSA | BB1 | 30 | WB × 5 | Pre | 25 | 29 | 28 | 23 |
| 4 | B | 5 | 4 | 0.2 | 0.1% BSA, | BB1 | 30 | WB × 10 | Pre, Post | 14 | 18 | 22 | 22 |
| | | | | | 5% HS | | | | | | | | |
| 5 | B | 5 | 0.4 | 0.2 | 0.1% BSA, | BB1 | 30 | WB × 25 | Pre, Post | 15 | 17 | 14 | 17 |
| | | | | | 10% HS | | | | | | | | |
| 6 | B | 5 | 0.4 | 0.2 | 0.1% BSA, | BB1 | 30 | WB (+20% | Pre, Post | 12 | 15 | 12 | 14 |
| | | | | | 20% HS | | | HS) × 3, | | | | | |
| | | | | | | | | WB × 5 | | | | | |
| 7 | B | 1 | 0.4 | 0.3 | 0.1% BSA, | BB1 | 30 | WB (+50% | Pre, Post | 13 | 16 | 16 | 18 |
| | | | | | 50% HS | | | HS) × 3, | | | | | |
| | | | | | | | | WB × 10 | | | | | |
| 8 | B | 1 | 0.04 | 0.6 | 0.1% BSA, | BB1 | 30 | WB (+50% | Pre, Post | 20 | 21 | 23 | 23 |
| | | | | | 50% HS | | | HS) × 3, | | | | | |
| | | | | | | | | WB × 10 | | | | | |
| 9 | C | 1 | 0.167 | 1 | 0.1% BSA | BB1 | 30 | WB (+2 M | Pre, Post | 28 | 22 | 24 | 19 |
| | | | | | | | | urea) × 3, | Total | 155 | 168 | 167 | 166 |
| | | | | | | | | WB × 2 | | | | | |

ExSELEX-2

| | | DNA | Target | Volume | | Binding | | | Counter | PCR cycles | | | |
|---|---|---|---|---|---|---|---|---|---|---|---|---|---|
| Round | Method | [nM] | [nM] | [mL] | Additives | Buffer | Time (min) | Washing | Selection | D1 | D2 | D3 | D4 |
| 1 | C | 500 | 5 | 8 | — | BB1 | 60 | BB1 × 3 | — | 20 | 20 | 20 | 20 |
| 2 | C | 100 | 5 | 1 | — | BB1 | 30 | BB1 × 5 | Pre | 22 | 22 | 19 | 20 |
| 3 | B | 50 | 2.5 | 0.4 | 0.1% BSA, | BB1 | 30 | WB × 5 | Pre | 15 | 21 | 25 | 21 |
| | | | | | 10% HS | | | | | | | | |
| 4 | B | 10 | 1 | 0.4 | 0.1% BSA, | BB1 | 30 | WB × 10 | Pre, Post | 20 | 24 | 25 | 19 |
| | | | | | 50% HS | | | | | | | | |
| 5 | C | 3 | 1 | 1 | 0.1% BSA | BB1 | 15 | BB1 (+3 M | Pre, Post | 26 | 20 | 27 | 19 |
| | | | | | | | | urea) × 3, | | | | | |
| | | | | | | | | BB1 × 2 | | | | | |
| 6 | B | 3 | 1 | 0.4 | 0.1% BSA, | BB1 | 30 | WB × 10 | Pre, Post | 18 | 20 | 24 | 16 |
| | | | | | 50% HS | | | | | | | | |
| 7 | C | 3 | 1 | 1 | 0.1% BSA | BB1 | 15 | BB1 (+3 M | Pre,Post | 24 | 18 | 27 | 18 |
| | | | | | | | | urea) × 3, | | | | | |
| | | | | | | | | BB1 × 2 | | | | | |
| 8 | B | 1 | 0.5 | 0.4 | 0.1% BSA, | BB1 | 30 | WB × 10 | Pre, Post | 23 | 23 | 25 | 21 |
| | | | | | 50% HS | | | | | | | | |
| 9 | B | 1 | 0.5 | 0.4 | 0.1% BSA, | BB1 | 30 | WB × 20 | Pre, Post | 23 | 24 | 27 | 21 |
| | | | | | 50% HS | | | | | | | | |
| 10 | D | 20 | 10 | 0.02 | — | BB1 | 30 | — | — | 12 | 12 | 12 | 12 |
| | | | | | | | | | Total | 203 | 204 | 231 | 187 |

ExSELEX-3

| | | DNA | Target | Volume | | Binding | | | Counter | PCR cycles | | |
|---|---|---|---|---|---|---|---|---|---|---|---|---|
| Round | Method | [nM] | [nM] | [mL] | Additives | Buffer | Time (min) | Washing | Selection | D1 | D2 | D3 |
| 1 | B | 2500 | 5 | 0.8 | 0.1% BSA, | BB2 | 30 | WB × 3 | — | 21 | 22 | 20 |
| | | | | | 10% HS | | | | | | | |
| 2 | B | 250 | 5 | 0.3 | 0.1% BSA, | BB2 | 30 | WB × 5 | Pre | 15 | 20 | 21 |
| | | | | | 10% HS | | | | | | | |
| 3 | B | 50 | 5 | 0.3 | 0.1% BSA, | BB2 | 30 | WB × 5 | Pre | 15 | 15 | 15 |
| | | | | | 20% HS | | | | | | | |
| 4 | B | 5 | 1 | 0.3 | 0.1% BSA, | BB2 | 30 | WB (+2 M | Pre | 24 | 27 | 23 |
| | | | | | 45% HS | | | urea) × 3, | | | | |
| | | | | | | | | WB × 2 | | | | |
| 5 | B | 1 | 0.2 | 0.3 | 0.1% BSA, | BB2 | 10 | WB (+2 M | Pre | 24 | 25 | 28 |
| | | | | | 45% HS | | | urea) × 3, | | | | |
| | | | | | | | | WB × 2 | | | | |
| 6 | B | 1 | 0.2 | 0.3 | 0.1% BSA, | BB2 | 5 | WB (+50% | Pre | 23 | 20 | 27 |
| | | | | | 45% HS | | | HS) × 2, | | | | |
| | | | | | | | | WB (+2 M | | | | |
| | | | | | | | | urea) × 2, | | | | |
| | | | | | | | | WB × 2 | | | | |
| 7 | B | 0.5 | 0.2 | 0.3 | 0.1% BSA, | BB2 | 5 | WB (+50% | Pre | 25 | 25 | 29 |
| | | | | | 45% HS | | | HS) × 3, | Total | 147 | 154 | 163 |
| | | | | | | | | WB (+3 M | | | | |
| | | | | | | | | urea) × 3, | | | | |
| | | | | | | | | WB × 3 | | | | |

Separation of DNA-target complexes (Method):
A: Ultrafiltration (Amicon Ultra-100kDa)
B: Sandwich (Capture with mAb#D06, in 96-well plates)
C: Complex immobilization (Dynabeads ™ His-Tag Isolation and Pulldown)
D: Separation by gel-mobility shift [4% PAGE (29:1 acrylamide-bisacrylatmide) supplemented with 5% glycerol and 2 M urea]
Buffers:
BB1: 20 mM Tris-HCl (pH 7.5), 150 mM NaCl, 1 mM MgCl₂, 2.7 mM KCl, 0.005% Nonidet-P40
BB2: 20 mM Tris-HCl (pH 7.5), 150 mM NaCl, 1 mM MgCl₂, 2.7 mM KCl, 2% Tween 20
WB: 20 mM Tris-HCl (pH 7.5), 150 mM NaCl, 1 mM MaCl₂, 2.7 mM KCl, 0.05% Tween 20

ExSELEX was performed targeting each recombinant DEN-NS1 serotype protein, as follows: DEN1-NS1 (D1), DEN2-NS1 (D2), DEN3-NS1 (D3), and DEN4-NS1 (D4) in the column of PCR cycles. To increase the stringency of the selection conditions, human serum (HS) was added to the binding buffer (additives) and urea in the washing buffer in later rounds.

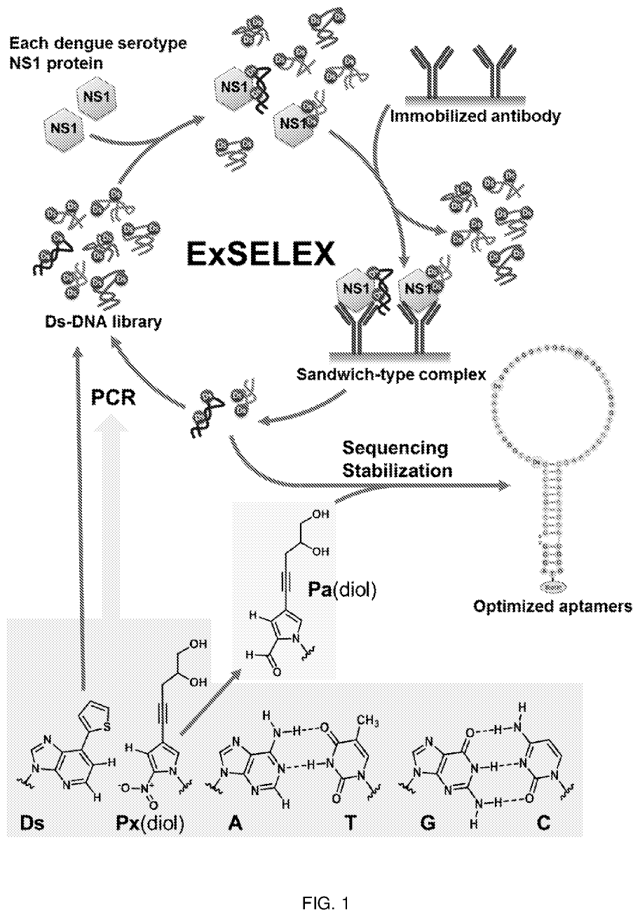

Four DEN-NS1 serotypes were purchased from The Native Antigen Company (Oxford, UK). In the ExSELEX procedure, a selection method using an anti-DEN-NS1 monoclonal antibody (Ab#D06) was employed (FIG. 1), which binds to all four serotypes of DEN-NS1 with 27-107 pM K_{D} values. The Ds-containing DNA library was mixed with each DEN-NS1 serotype, and then the NS1-DNA complexes were captured with immobilized Ab#D06 on a plate. The unbound DNA species were washed from the plate, and the DNA species bound on the plate were isolated and amplified by PCR for subsequent rounds of selection.

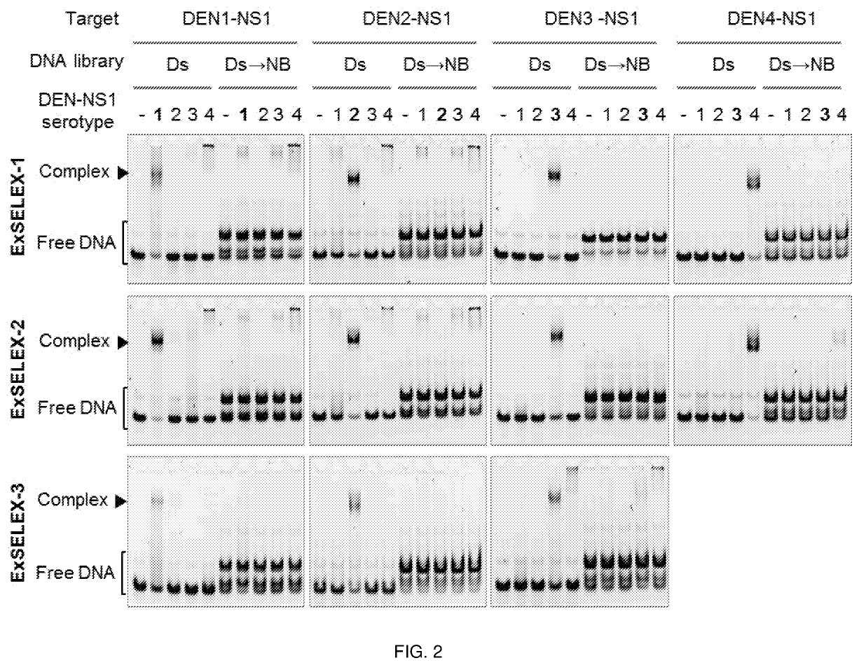

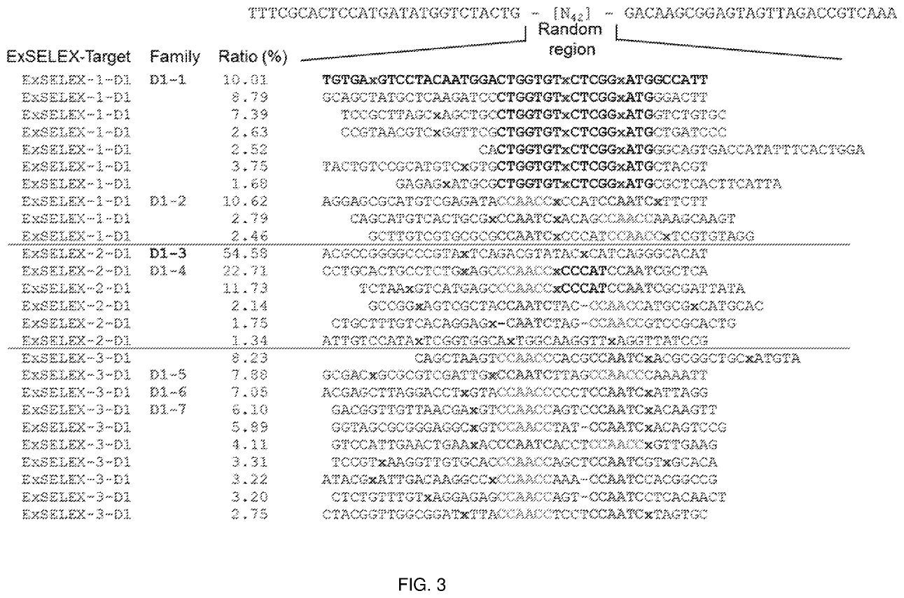

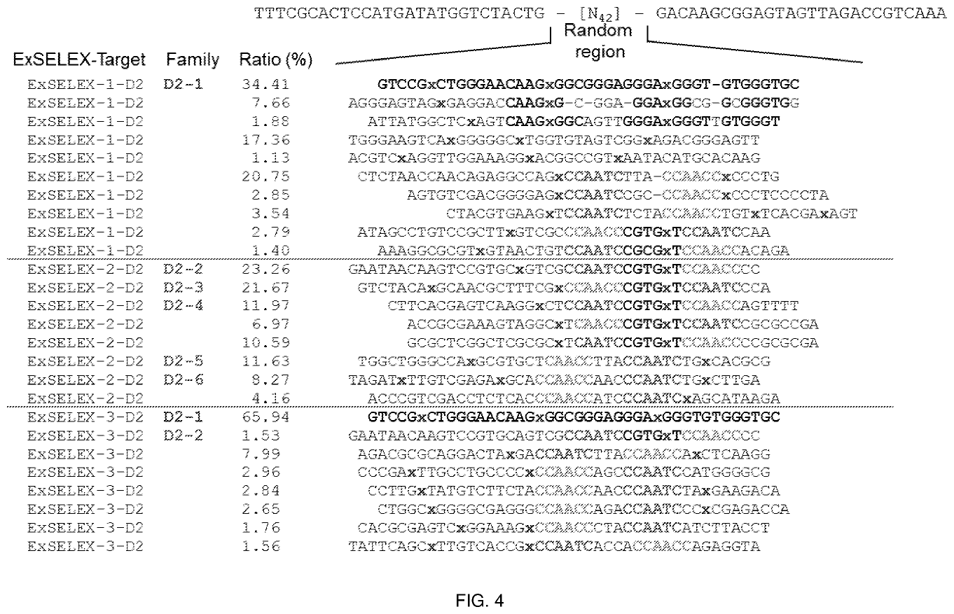

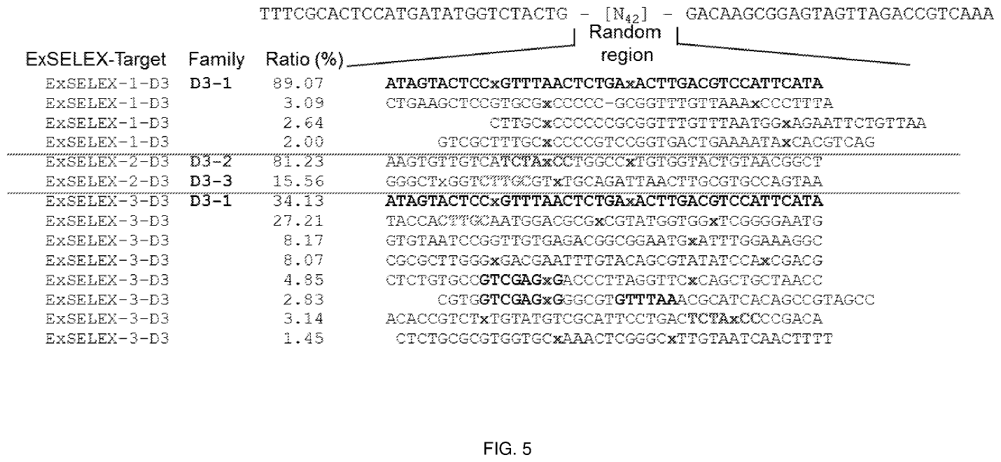

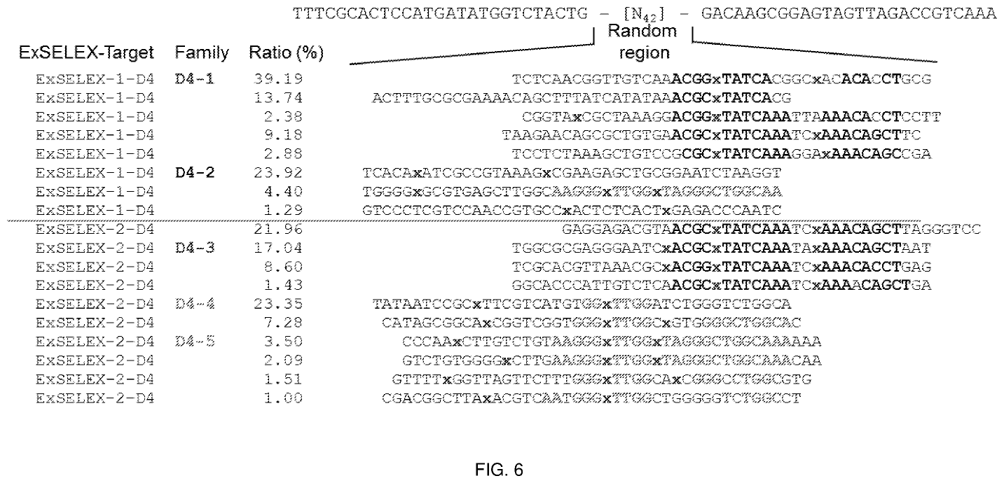

After 7-10 rounds of selection, enriched DNA libraries were obtained and their high specificities to each DEN-NS1 serotype were confirmed by electrophoresis gel-mobility shift assays (EMSAs) (FIG. 2). The high Ds dependency was also confirmed by EMSAs using library variants with the Ds→natural base mutations³⁹, which did not form clearly discernible complexes with any DEN-NS1 proteins (FIG. 2). The sequences in the enriched DNA libraries (FIGS. 3-6) were determined by sequencing methods^{34,39}, from which several aptamer candidates for each serotype were selected (Table E2).

TABLE E2
Sequences of anti-DEN-NS1 DNA aptamer candidates.
In parentheses: EMSA using 2M urea gel.
Relative shifted ratio (%)−: <10%, +: 10-40%, ++: 40-60%, +++: >60%

| Original Name | Name | EMSA | SPR | | Sequence (5′- to -3′: L = Biotin-dT, = [symbol] dDs) |
|---|---|---|---|---|---|
| [symbol] | [symbol] | (++) +++ | | specific | [symbol] |
| | | | 132 pM | | |
| [symbol] | [symbol] | +++ | K_{D} = | specific | [symbol] |
| | | | 197 pM | | |
| [symbol] | D1-1-46h | +++ | [symbol] | specific | [symbol] |
| | | | [symbol] | | |
| [symbol] | [symbol] | | − | [symbol] | [symbol] |
| Biol4D1Aib | D2-1-78 | (+) | | | [symbol] |
| (78-mer) | | | | | |
| Biol5D1a02 | D1-3-78 | (+++) +++ | K_{B} = | non- | LTGATATGGTCTACTGACGCCGGGGCCCGTAxTCAGACGTATACx |
| (78-mer) | | | 55 pM | specific | CATCAGGGCACATACAAGCGGAGTAGTTAGACC |
| 15D1A02h | D1-3-47 | +++ | K_{D} = | non- | [symbol] |
| (47-mer) | | | 98 pM | specific | |
| Biol5D1A01 | D1-4-78 | (−) | | | [symbol] |
| (78-mer) | | | | | |
| 16D1-2h | D1-5-61h | ++ | | | [symbol] |
| (61-mer) | | | | | |
| 16D1-3h | D1-6-47h | ++ | | | [symbol] |
| (47-mer) | | | | | |
| 16D1-4h | D1-7-51h | ++ | | | [symbol] |
| (51-mer) | | | | | |
| | | | | | Sequence (5′- to -3′: 1 = Biotin, L = |
| | | | | | Biotin-dT, x = dDs, [symbol] = Diol1=dPa, |
| Original Name | Name | EMSA | SPR | | y = Diol1-dPx) |
| [symbol] | D2-1-78 | (−) | | | [symbol] |
| 14D2A1-96 | D2-1-96 | − | | | [symbol] |
| | [symbol] | | | | |
| [symbol] | D2-1d-97 | ++ | [symbol] | | [symbol] |
| [symbol] | D2-1y-96 | +++ | K_{b} = | | [symbol] |
| | | | 41 pM | | |
| [symbol] | D2-1d-64 | + | | | [symbol] |
| [symbol] | D2-1d-74 | ++ | | | [symbol] |
| [symbol] | D2-1d- | ++ | K_{b} = | specific | [symbol] |
| | 87h | | 105 pM | | |
| [symbol] | [symbol] | ++ | | | [symbol] |
| [symbol] | [symbol] | ++/+++ | [symbol] | specific | [symbol] |
| AptD2b | [symbol] | ++/+++ | | | [symbol] |
| AptD1c | D2-Dd- | − | | | [symbol] |
| | 72h-c | | | | |
| AptD2d | D2-1d- | − | | | [symbol] |
| | 72b-d | | | | |
| AptD2e | Cont-D2- | − | | | [symbol] |
| | [symbol] | | | | |
| [symbol] | [symbol] | − | | | [symbol] |
| [symbol] | D2-D1- | +++ | | | [symbol] |
| | 62h | | | | |
| [symbol] | [symbol] | − | | | [symbol] |
| [symbol] | D2-2-78 | (−) | | | [symbol] |
| 15DCAixh | D2-2d- | + | | | [symbol] |
| (59-mer) | 59h | | | | |
| Biol15D2A03 | D3-3-78 | (−) | | | [symbol] |
| (78-mer) | | | | | |
| 15D2A3xh | D3-3d-52h | ++ | | | [symbol] |
| (52-mer) | | | | | |
| Biol15D2A02 | D2-4-79 | (−) | | | [symbol] |
| (78-mer) | | | | | |
| 15DCA2xh | [symbol] | + | | | [symbol] |
| (56-mer) | | | | | |
| 15D2A6ah | D2-5-96h | + | | | [symbol] |
| (46-mer) | | | | | |
| 15D2A6bh | D5-5-48h | + | | | [symbol] |
| (48-mer) | | | | | |
| [symbol] | D2-6-59h | − | | | [symbol] |
| (54-mer) | | | | | |
| | | | | | Sequence (5′- to -3′: |
| Original Name | Name | EMSA | SPR | | L = Biotin-dT, z = dDs) |
| Biol14D3A01 | D3-1-85 | (++) | | | [symbol] |
| (65-mer) | | | | | |
| [symbol] | D3-2-76 | (+++) | [symbol] | specific | [symbol] |
| (78-mer) | | +++ | | | |
| [symbol] | [symbol] | +++ | [symbol] | specific | [symbol] |
| [symbol] | [symbol] | − | [symbol] | | [symbol] |
| Biol15D3A02 | [symbol] | (+) | | | [symbol] |
| (78-mer) | | | | | |
| | | | | | Sequence (5′- to -3′: |
| Original Name | Name | EMSA | SPR | | L = Biotin-dT, x - dDs) |
| Biol14D4A01 | D4-1-78 | (+++) | Kₚ = | specific | [symbol] |
| (78-mer) | | +++ | 62 pM | | |
| 14D4Alah | D4-1-57h | +++ | Kₚ = | specific | [symbol] |
| (52-mer) | | | 29 pM | | |
| Biol14D4A02 | D4-2-78 | (+++) | | | [symbol] |
| (78-mer) | | | | | |
| [symbol] | D4-3-78 | (+++) | Kₚ = | specific | [symbol] |
| | | +++ | 34 pM | | |
| [symbol] | [symbol] | +++ | [symbol] | specific | [symbol] |
| [symbol] | [symbol] | [symbol] | [symbol] | | [symbol] |
| [symbol] | D4-478 | (+++) | | | [symbol] |
| Biol14D4A05 | D4-5-78 | (++) | | | [symbol] |
| (78-mer) | | | | | |

[symbol]

indicates data missing or illegible when filed

In Table E2, the oligonucleotide sequences used for the binding analyses against each target DEN-NS1, are summarized with the results of the electrophoresis gel-mobility shift assay (EMSA) and surface plasmon resonance (SPR) analysis. The additional complementary sequences that form stems are underlined. The oligonucleotides containing a mini-hairpin sequence, CGCG-(Biotin-T)-AGCG, at the 3′-terminus have an additional “h” in the aptamer candidate names. In the SPR analysis with 20 nM of each dengue NS1 protein, “specific” means that the oligonucleotide only bound to the target serotype DEN-NS1, and not to the other serotype DEN-NS1, while “less-specific” means the oligonucleotide exhibited binding to not only target serotype NS1 but also to some of the other serotype NS1 proteins. The chemical structures of the unnatural bases, diol-Px (Px) and diol-Pa (Pa), referenced in the table are shown below.

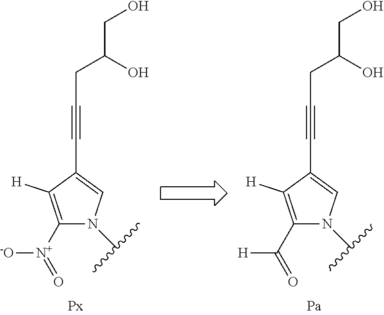

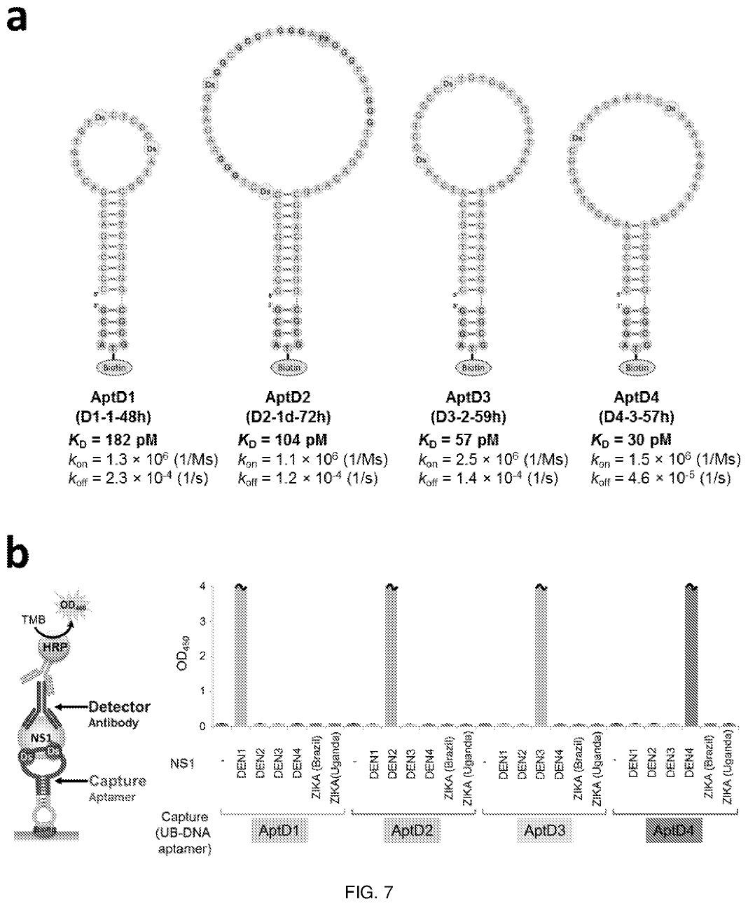

Most of the sequences contained complementary motifs at the 5′- and 3′-regions, and thus the complementary motifs were trimmed to form a clear stem structure. To increase the thermal and enzymatic stabilities of the aptamer candidates, a specific biotin-conjugated DNA sequence (mini-hairpin DNA)⁴⁰⁻⁴², CGCG(Biotin-T)AGCG, was added at their 3′-termini^{38,43,44} (FIG. 7a). The aptamer candidates and their variants were chemically synthesized for further experiments.

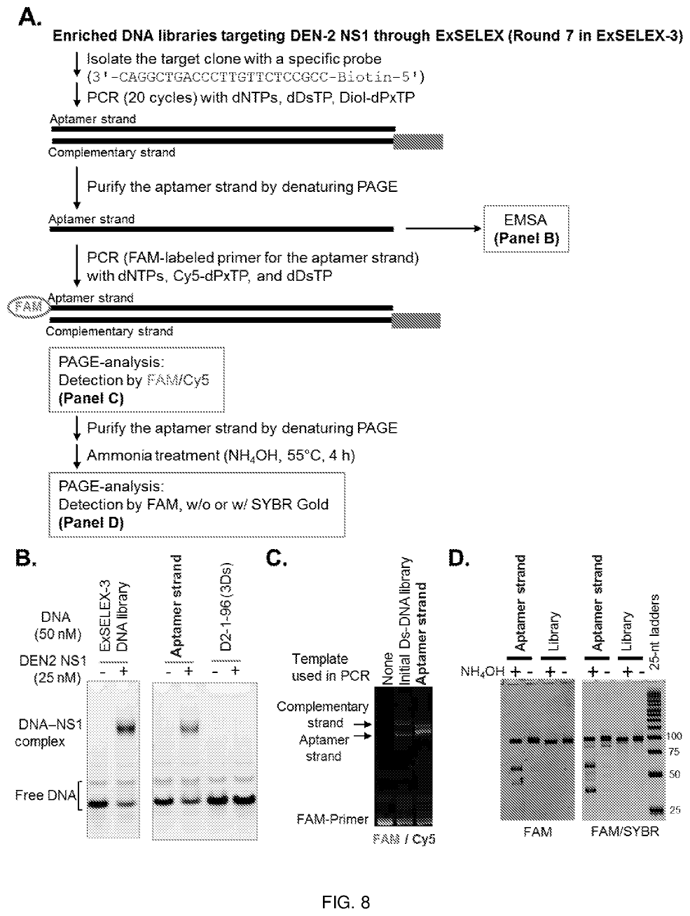

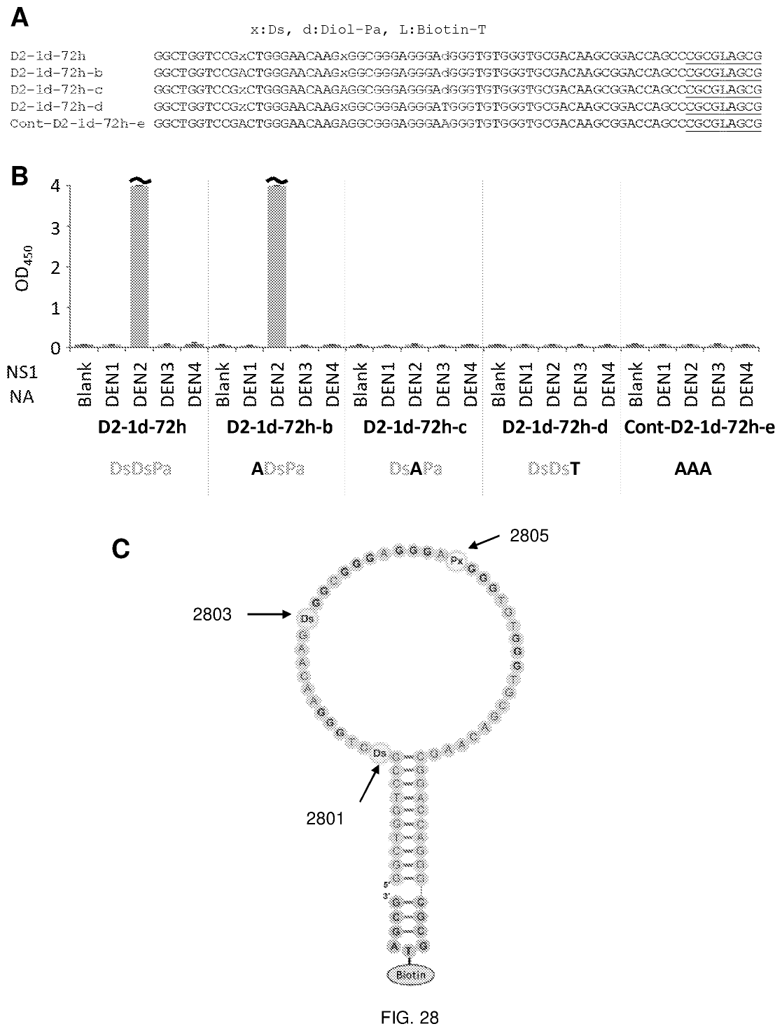

A notable case involved some candidates obtained by ExSELEX targeting DEN2-NS1. One of the sequence families (D2-1), which exhibited the highest affinity to DEN2-NS1, contained two Ds and one Px bases (refer to FIG. 8 and methods for the sequence determination involving the Px base). The additional Px in the aptamers resulted from the mutation of the natural bases to Px during PCR amplification in ExSELEX³⁴. The Px-containing DNA fragments cannot be chemically synthesized, because the Px nucleoside degrades under the basic synthesis conditions. This instability of the Px nucleoside results from the combination of the nitro group and pyrrole ring of Px⁴⁵, and thus the nitro group was replaced with an aldehyde group (Pa, pyrrole-2-carbaldehyde)^{46,47} (FIG. 1 and Table E2). The amidite derivative of the diol-conjugated Pa nucleoside was newly synthesized for DNA chemical synthesis, and several D2-1 variants containing Ds and Pa were synthesized by the standard phosphoramidite method. The Px→Pa aptamer variants still retained the high affinity and specificity to DEN2-NS1. This may be attributed to the similarity between the two unnatural bases. Among other similarities, both unnatural bases contain a diol group. Finally, AptD2 (D2-1d-72 h) was developed, containing two Ds and one Pa bases, as a DEN2-NS1 binder. When the Ds base at the 11^{th} position (or position 2801 in FIG. 28) was replaced with a natural alanine base, the aptamer retained its high affinity and specificity to DEN2-NS1. However, when the Ds base at the 23^{rd} position (or position 2803 in FIG. 28) was replaced with a natural alanine base, the binding affinity of the aptamer to DEN2-NS1 was abolished. Similarly, when the diol-Pa/diol-Px base at the 35th position (or position 2805 in FIG. 28) was replaced with a natural thymine base, loss of binding was observed (FIG. 28). In some examples, when the diol-Pa/diol-Px base was replaced with a Ds base, loss of binding was also observed (see e.g. Table E2). Thus, the Ds base at the 23^{rd} position and the diol-Pa/diol-Px base at the 35^{th} position contribute to the aptamer's binding affinity for DEN2-NS1. The results also suggest that base variation at the 11^{th} position or substituition of the Ds base at the 11^{th} position may be tolerated.

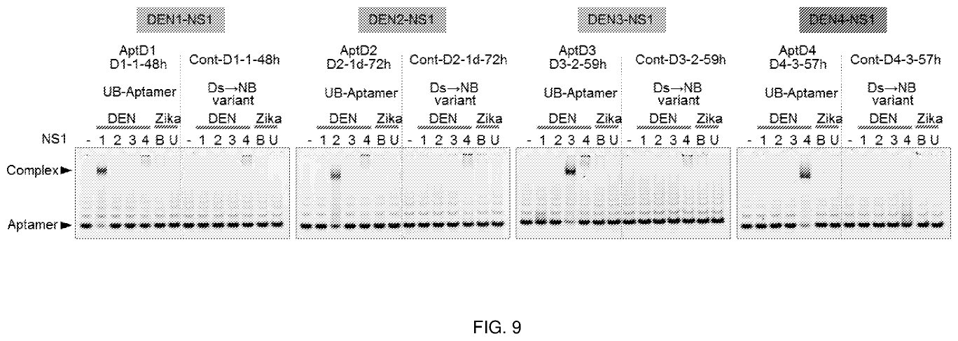

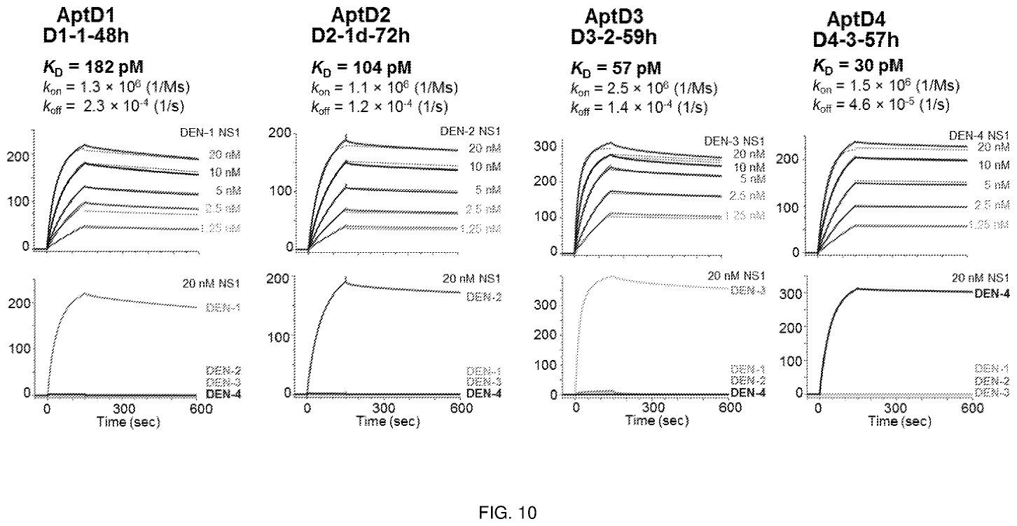

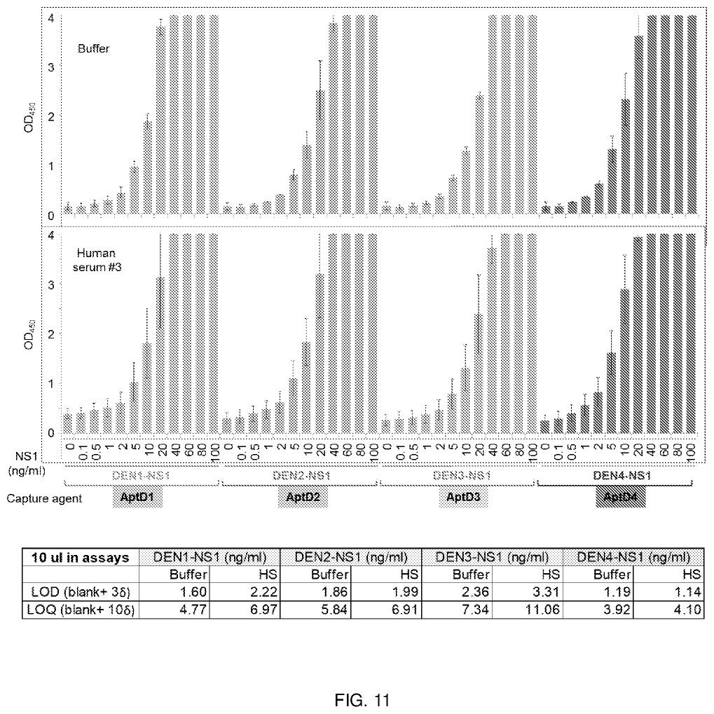

Each aptamer sequence was finalized by adding the biotin-conjugated mini-hairpin sequence at its 3′-terminus (AptD1 (D1-1-48 h) for DEN1-NS1, AptD2 (D2-1d-72 h) for DEN2-NS1, AptD3 (D3-2-59 h) for DEN3-NS1, and AptD4 (D4-3-57 h) for DEN4-NS1) (FIG. 7a). The high specificity of each aptamer to its serotype-specific DEN-NS1 was confirmed by EMSA and surface plasmon resonance (SPR) analyses (FIGS. 9 and 10). The natural-base variants, in which the UBs were replaced with natural bases, significantly reduced their affinities to each target, indicating the necessity of these UBs for the aptamers' binding capabilities. The K_{D} values of AptD1, AptD2, AptD3, and AptD4 to each target DEN-NS1 were 182, 104, 57, and 30 pM, respectively.

The detection of each DEN-NS1 serotype was examined by a sandwich-type ELISA format, using the antibody Ab#D06 as the primary detector agent and the aptamers as capture agents (FIG. 7b). The signal was detected by the colorimetric output, using a secondary anti-IgG HRP-conjugated antibody. Each aptamer specifically detected its target DEN-NS1 serotype, and no cross-reactivities with nontarget DEN-NS1 serotypes or Zika NS1 proteins were observed. The limit of detection (LOD) in buffer was 1.19-2.36 ng/ml for each DEN-NS1 (FIG. 11).

### Serotype-Specific Detection of DEN-NS1 in Patient Samples

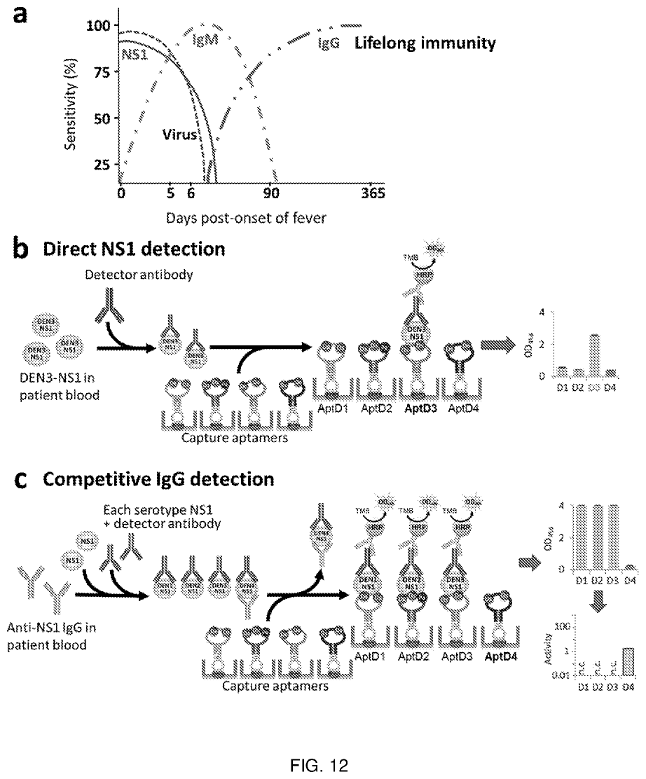

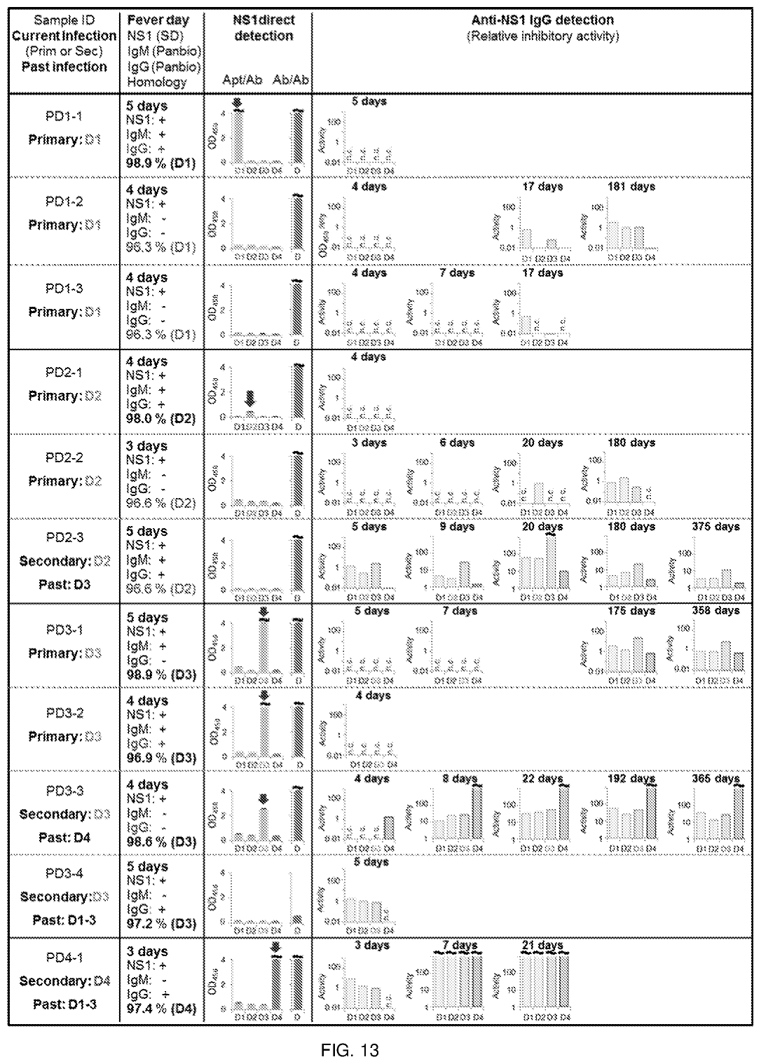

Using blood samples from eleven Singaporean patients (PD1-1-PD4-1) with acute DENV infection, the sensitivity and specificity of the ELISA format to detect each DEN-NS1 serotype were evaluated (FIGS. 12b and 13). The serotype of the current infection in each patient sample was also determined by RT-qPCR and sequencing (FIG. 13). The ELISA format detected each DEN-NS1 serotype in the PD1-1, PD2-1, PD3-1, PD3-2, PD3-3, and PD4-1 serum samples. However, PD1-2, PD1-3, PD2-2, PD2-3, and PD3-4 could not be detected, although the ELISA format using an antibody—antibody sandwich pair (Ab#D06 and Ab#D25 (1.6-138 pM K_{D} values for DEN-NS1)) and the commercial LFA system (SD BIOLINE) detected DEN-NS1 in all of the samples (FIG. 13), except for PD3-4, which was not detected by the antibody-antibody pair (discussed below).

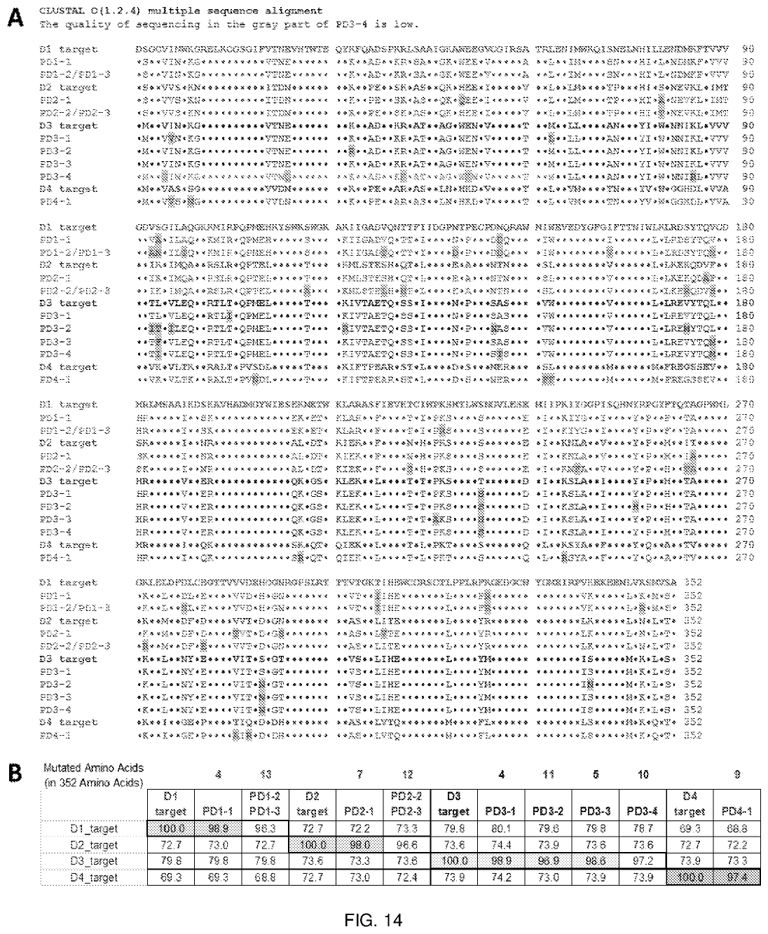

The false-negative results of PD1-2, PD1-3, PD2-2, and PD2-3 were caused by the subtle amino acid differences between the DEN-NS1 present in the samples and those in the DEN-NS1 purchased from The Native Antigen Company, which were used as the targets for the aptamer generation. The amino acid sequences of DEN-NS1 in the patient samples were determined, and many amino acid substitutions were found when compared to those of the target NS1 proteins (FIG. 14). The sequence data revealed that the aptamers could bind specifically to the target DEN-NS1 with amino acid sequence homology of at least 96.9%. The DEN1-NS1 and DEN2-NS1 detection clearly showed the relationship between the homology and the aptamer affinity. The DEN1-NS1 of PD1-1 had 98.9% homology with that of The Native Antigen Company, as detected by ELISA using AptD1. In contrast, PD1-2 and PD1-3 had 96.3% homologies with that from The Native Antigen Company, and were not detected with AptD1. Similarly, the homologies of the DEN2-NS1 of PD2-1, PD2-2, and PD2-3 to that of The Native Antigen Company were 98.0, 96.6, and 96.6%, respectively, and AptD2 detected only the PD2-1 sample. For DEN3-NS1 and DEN4-NS1, the homologies were 96.9-98.9% and AptD3 and AptD4 bound to each DEN-NS1.

### Serotype-Specific Detection of Anti-DEN-NS1 IgG Antibodies in Patient Samples

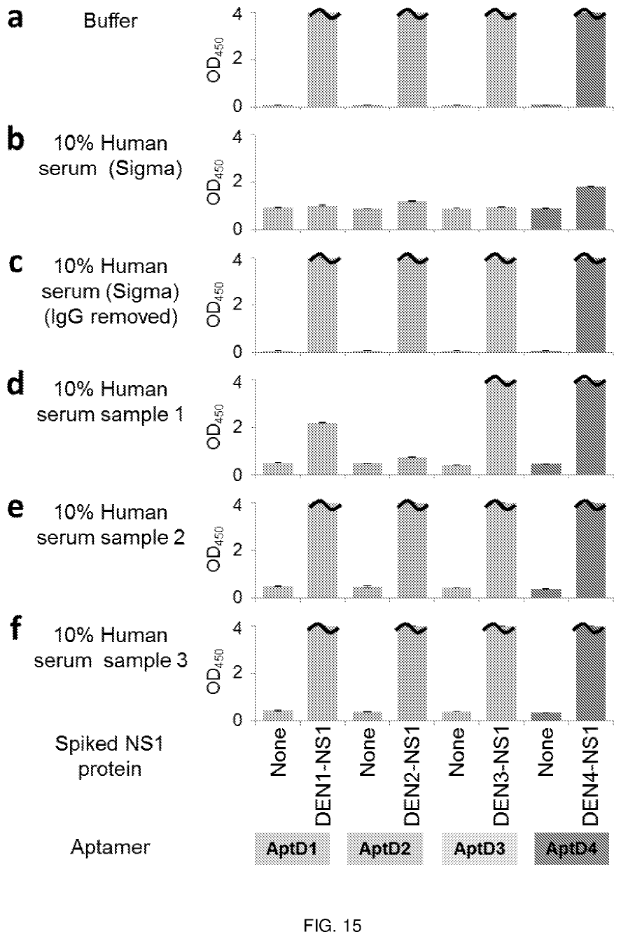

Using the ELISA format, it was found that it can also be used for the detection of serotype-specific anti-NS1 IgG antibodies in patient serum samples. When the ELISA sensitivity of the aptamer-antibody pair for DEN-NS1 detection in the presence of human serum purchased from Sigma-Aldrich was examined, the detection was significantly inhibited (FIG. 15b), relative to that in buffer (FIG. 15a). One of the plausible causes is the presence of anti-DEN-NS1 IgG antibodies in the serum, which inhibited the binding of the aptamer to the additional NS1 proteins. To validate this contamination theory, the total IgG antibodies were removed from the serum by treating it with protein A-immobilized resin, and confirmed the absence of inhibition with the treated serum (FIG. 15c). An ELISA was also performed using a serum sample from a Singaporean who was not infected with dengue at the time, to determine whether the serum inhibited the detection. Interestingly, the serum showed the serotype-specific inhibitions in the DEN2-NS1 detection, as well as DEN1-NS1 to some extent (FIG. 15d), suggesting that the person might have previously been infected with the dengue serotype 2 and/or serotype 1 viruses. Therefore, two other serum samples were obtained from a dengue non-endemic country, and performed an ELISA. As expected, the two serum samples did not inhibit the DEN-NS1 detection in the ELISA format (FIGS. 15e and 15f). These results inspired us to develop a new method for the serotype-specific detection of anti-DEN-NS1 IgG antibodies in human serum samples (FIG. 12c), as well as for the direct DEN-NS1 detection (FIG. 12b).

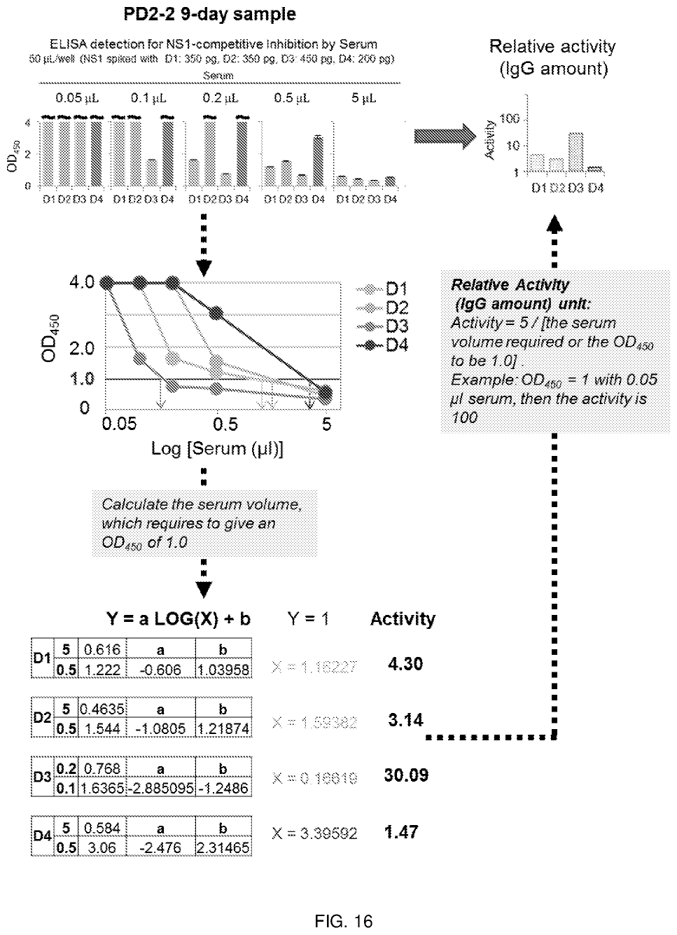

For the serotype-specific IgG detection, a simple quantification method was developed for the anti-DEN-NS1 IgG activities (FIG. 16). To this end, competitive-inhibition ELISA was performed using a series of different volumes (0.05, 0.1, 0.2, 0.5, and 5 μl) of patient serum, in the presence of a certain amount of each serotype DEN-NS1 (The Native Antigen Company). After the absorbance measurement at 450 nm (OD₄₅₀) in the ELISA format, the OD₄₅₀ values were plotted against the volume of serum, and the serum volume required to give an OD₄₅₀ of 1.0 was calculated. The relative IgG activity (Activity) was then defined by the following formula: Activity=5/(the serum volume required for an OD₄₅₀ of 1.0).

Using this competitive ELISA format and quantification method, the longitudinal changes in the IgG production and the serotype specificities of the patient samples were measured (FIG. 13). Even in the recovered patients after one year, the IgG antibodies were detectable (PD2-3, PD3-1, and PD3-3). Furthermore, the method clearly identified the primary and secondary infections. The samples can be categorized into two groups by the IgG detection: one group included PD1-1, PD1-2, PD1-3, PD2-1, PD2-2, PD3-1, and PD3-2, in which the IgG was not detected within a week after fever onset, and the other group included PD2-3, PD3-3, PD3-4, and PD4-1, in which the IgG was detected in 3-5 days. The data suggested that the latter patients were previously infected by dengue. There were some discrepancies in PD1-1, PD3-2, and PD3-3 between the IgG detection and the conventional LFA method (Panbio) (FIG. 13). The visual judgement using the LFA format was often ambiguous, and all of the longitudinal IgG detection data supported the higher accuracy of the present method over that of the LFA format. Thus, it was concluded that the first group most likely represented the primary infection, and the second group was a secondary or higher infection. The IgG detection can identify the primary or secondary infection of patients within 3-5 days after fever onset. In addition, in each primary infected patient sample, the infected serotype determined by RT-qPCR is identical to the serotype showing the highest activity among the detected IgG antibodies in the competitive ELISA system. The competitive ELISA method is specific to IgG. In the samples of patients with the primary infection, IgM was detected in PD1-1, PD2-1, PD3-1, and PD3-2 by LFA (Panbio). However, no inhibitions of the DEN-NS1 detections in the competitive ELISA were detected within the first week of the fever onset, and the inhibitions were detected at 17 days or thereafter (FIG. 13). Thus, the aptamer binding was not inhibited by the IgM produced in the early phase of the infection (FIG. 12a).

The quantitative serotype analysis of PD2-3, PD3-3, and PD4-1 revealed that the initial IgG level reflected mainly the serotypes of the past infection. Even after one week, the production of the IgG antibodies that predominantly recognized the serotype resulting from the past infection increased sharply, as compared to the IgGs produced from the current secondary infection. Although the predominance of the past infection varied depending on the patient, the PD2-3 and PD3-3 patient samples revealed the massive production of the IgG antibodies to the past serotype infection.

As mentioned above, DEN3-NS1 of PD3-4 was not detected by ELISA, using both the antibody-aptamer and antibody-antibody (Ab#D06-Ab#D25) sandwich systems. This is because the serum sample already contained the anti-DEN3-NS1 IgG antibodies resulting from a past infection, which in turn inhibited the aptamer binding, as well as the Ab#D06 and/or Ab#D25 binding to DEN3NS1.

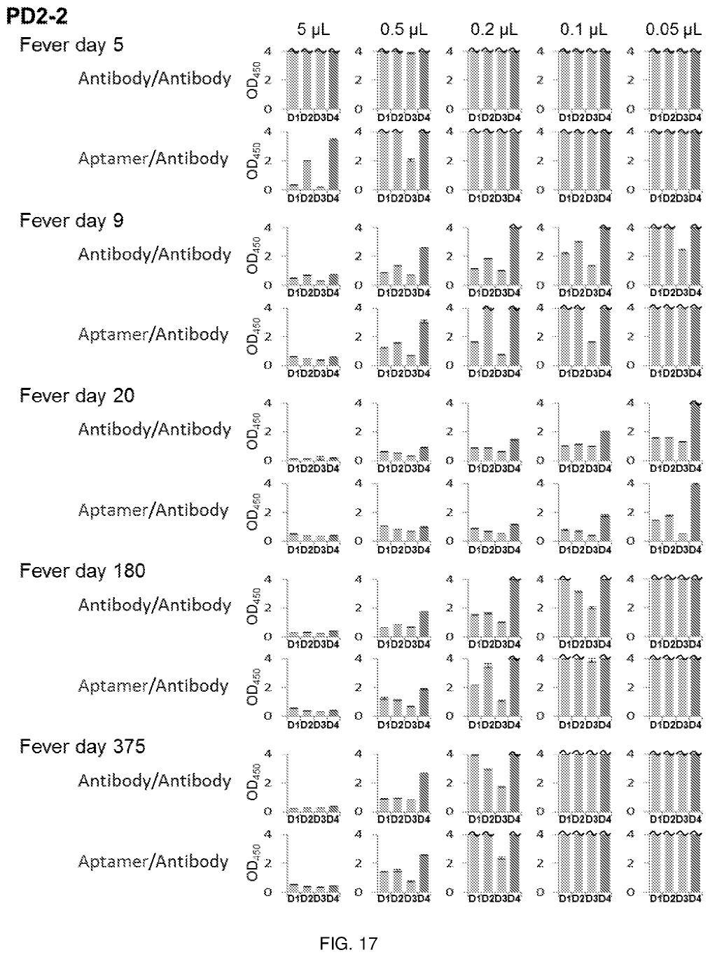

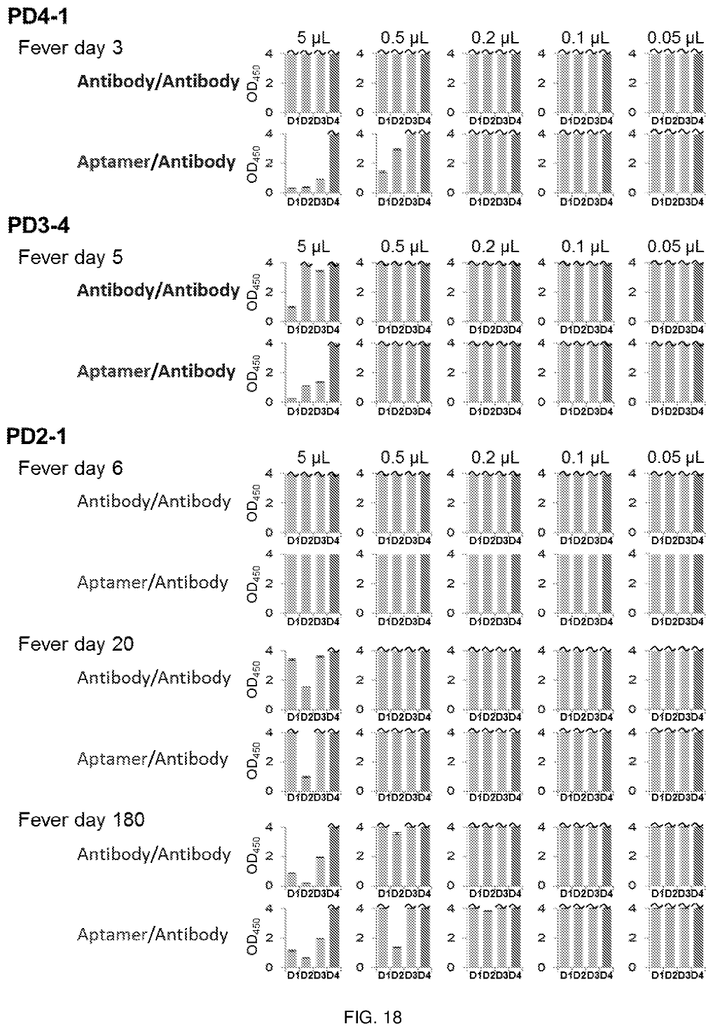

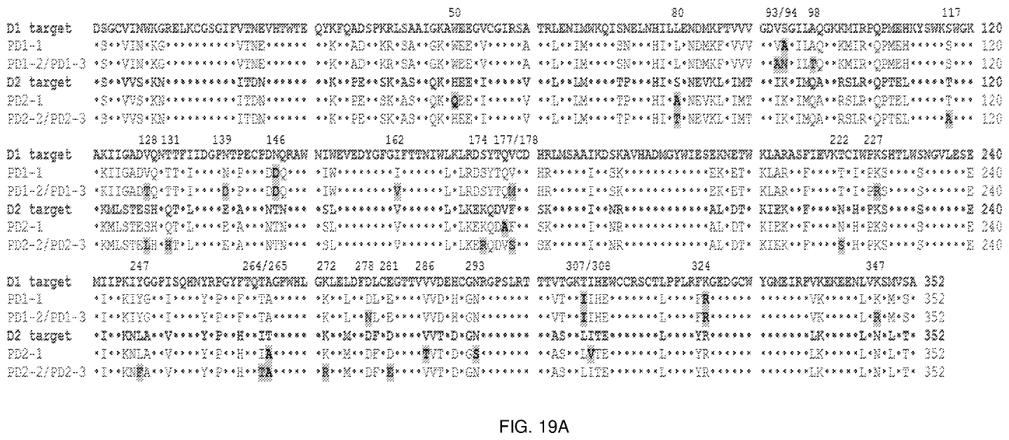

This IgG detection method using the aptamer-antibody sandwich pair exhibited higher sensitivity and serotype specificity, as compared to that using the antibody-antibody sandwich pair. To determine whether the antibody-antibody pair can also be used for IgG detection, the competitive inhibition in ELISA using the combination was compared with the antibody-antibody (Ab#D06-Ab#D25) pair for the patient sera with PD2-3, PD3-3, PD3-4, and PD4-1 (FIGS. 17 and 18). The DEN-NS1 ternary complex formation with the antibody-antibody sandwich pair was also inhibited by the anti-DEN-NS1 IgG in the patient serum. However, the antibody-antibody pair was not able to detect the IgG activities in the day 5 sample of PD2-3 and the day 3 sample of PD4-1. Overall, the serotype sensitivities and specificities of the aptamer-antibody pairs were higher than those of the antibody-antibody pair.

### Discussion

Presented herein are serotype-specific detection methods for DEN-NS1 and IgG in human serum, using high-affinity and high-specific UB-DNA aptamers. Among the generated UB-DNA aptamers, AptD2, which bound to DEN2-NS1, contained two Ds and one Px bases as the fifth and sixth bases. The high affinity of AptD2 to DEN2-NS1 indicates the importance of the diol group of Px/Pa for the binding. The combination of the hydrophobic Ds and the hydrophilic Px/Pa bases creates a new type of six-letter DNA aptamers with high affinity and specificity to their targets.

The specificities of these UB-aptamers are extremely high, and they recognize the target variants with amino-acid sequences that are at least 96.9% identical to that of the initial targets (purchased from The Native Antigen Company). This degree of homology is much higher than that among the different NS1 serotypes (69-80%). Due to their high specificity, AptD1 and AptD2 could not bind to some of the DEN1-NS1 (PD1-2/1-3) and DEN1-NS2 proteins (PD2-2/2-3) of the Singaporean patients.

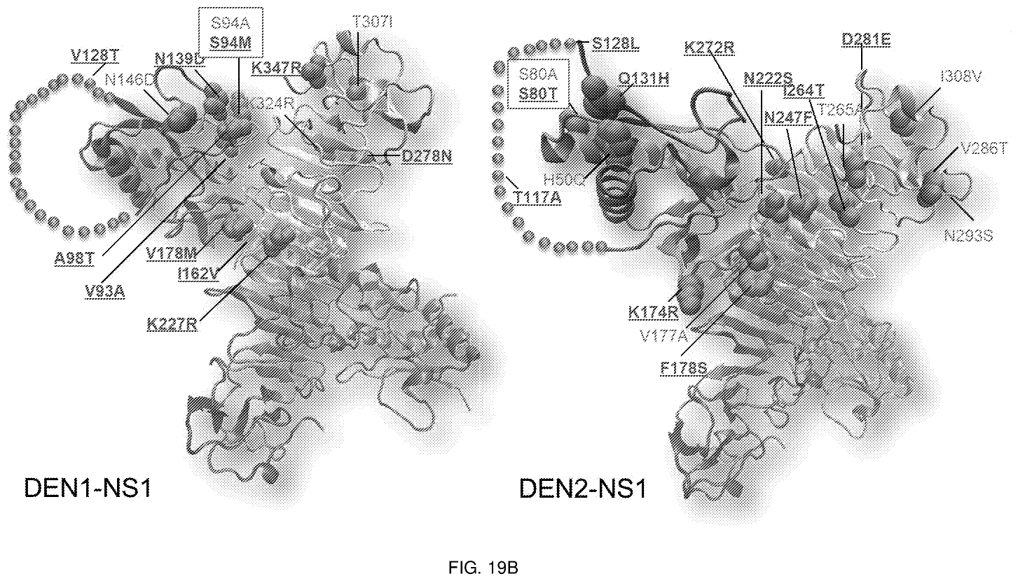

Remarkably, there are ten and eleven amino acid differences between the PD1-1 and PD1-2/1-3 DEN1-NS1 and between PD2-1 and PD2-2/2-3 DEN2-NS1 (352 amino acids), respectively. The locations of these amino acid differences suggest that they might participated in the aptamer binding site (FIG. 19); the substitution of nonpolar amino acids of PD1-1 and PD2-1 to other amino acids of PD1-2/1-3 and PD2-2/2-3 might facilitate interactions between the nonpolar amino acids and the hydrophobic Ds bases. The generation of a series of UB-aptamers corresponding to each variant of DEN-NS1 could open the door to rapid and precise diagnoses of DENV mutations beyond the serotype identifications used for pandemic surveys.

In contrast to the sensitive and direct DEN-NS1 detection, the present method for the serotype-specific IgG antibody detection can be used widely for DENV variants. To knowledge, this is the first simple method capable of identifying the IgG serotype specificities using DNA aptamers, although a direct IgG detection method by ELISA using antibodies has been reported³⁶. A similar IgG detection concept using conventional DNA aptamers was reported, to detect the IgG antibodies to the P48 protein of M. bovis⁴⁸. However, the affinities of the DNA aptamers to the target were relatively low (K_{D}=16-33 nM), and thus the background in the IgG detection was high and the quantitative analysis was difficult. The IgG detection provides valuable information for the dengue diagnostics and the use of dengue vaccine. The secondary infection can be identified by the IgG detection within several days (during the febrile period) after fever onset. If anti-DEN-NS1 IgG antibodies are detected in patients within one week after fever onset, then this indicates a secondary infection and may warrant close monitoring. Serotype specific IgG detection will also provide valuable information for the usage and analyses of the dengue vaccines, for which documentation of prior infection is important prior to administration, due to the concern of ADE.

The tests using patient samples with secondary DENV infections revealed that the IgG antibodies that responded to the past infection were predominantly produced, even upon secondary infections with different dengue serotypes. The results correlate with other reports^{11-14,16,17} and support ADE where secondary heterologous infections occasionally result in severe symptoms and why the vaccination of dengue-naive individuals is risky. Patients with a primary infection produced IgG antibodies that mainly targeted the infected serotype. In the secondary infection, the initially produced IgG antibodies reacted more to the NS1 serotype of the past infection, and did not effectively react with the targets of the secondary infection. The application of this test in a larger cohort of dengue patients will allow us to understand the mechanism of dengue pathogenesis, through the serotype-specific sequence of DENV infection. The present method may potentially be expanded to test the efficacy of vaccine development^{36,37}, and to diagnose other diseases and allergies.

High-Specificity Unnatural-Base DNA Aptamers that Selectively Distinguish Dengue NS1 Protein Variants with Several Amino Acid Mutations Beyond the Serotype Specificity

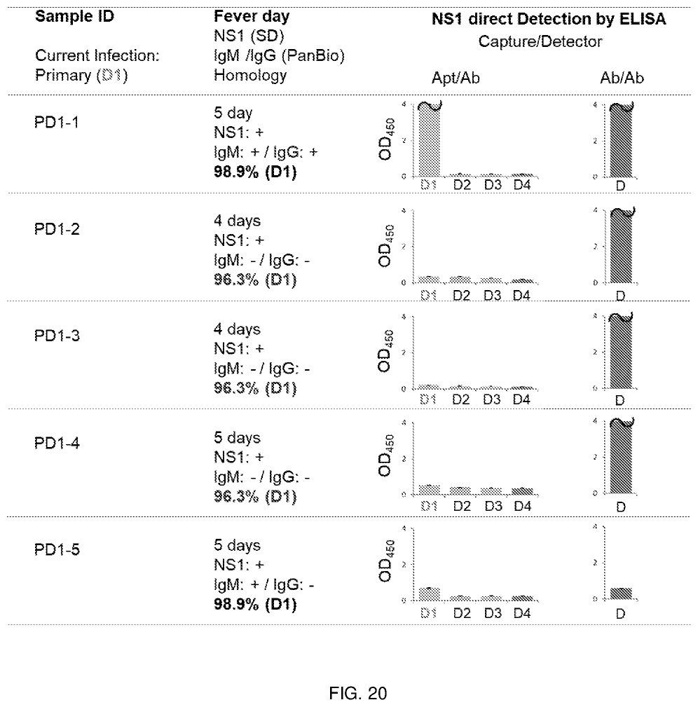

The foregoing described a series of unnatural-base-containing DNA (UB-DNA) aptamers that bind specifically to dengue NS1 protein variants with more than 96.9% amino-acid homologies to the initial targets (purchased from Native Antigen Company, NA) in each serotype of Singaporean patient serums. For example, one of the UB-DNA aptamers targeting the commercially available dengue serotype 1 NS1 protein detected only serotype 1 NS1 protein variants with more than 98.9% homologies in patient serums by the ELISA system (PD1-1 and PD1-5 in FIG. 20). Here, new UB-DNA aptamers that bind specifically to other variants of dengue serotype 1 NS1 proteins with 96.3% homologies in patient serums (PD1-2, 1-3, and 1-4) were generated.

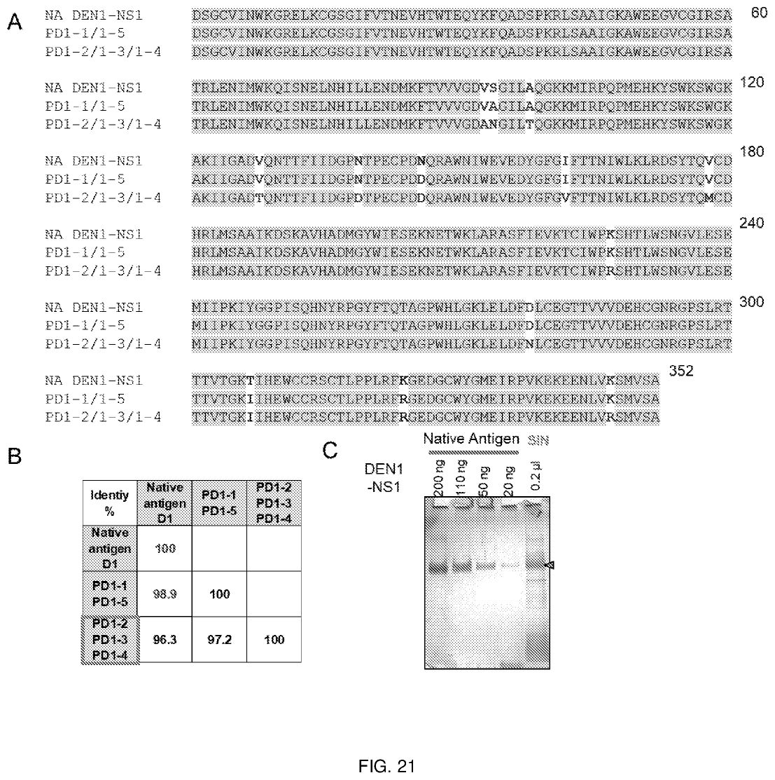

The amino acid sequences of the dengue serotype 1 NS1 protein variants in the patient serums are the same (PD1-2, 1-3, and 1-4 in FIGS. 21A and 21B), in which 13 residues were mutated among the 352 amino-acid residues in the full-length protein. Thus, the recombinant NS1 protein variant (SIN DEN1-NS1) was prepared the in-house. The NS1 region of the cDNA obtained from another patient sample, which also encoded the same amino acid sequence with PD1-2, 1-3, and 1-4, was sub-cloned to an in-house expression vector generally used for rabbit monoclonal antibody expression. The SIN DEN1-NS1 proteins with a six-histidine tag at the C-terminus were expressed in cultured CHO cells, and purified by the histidine-tag pull-down method. The purities of the obtained SIN DEN1-NS1 proteins were analyzed by SDS-PAGE with silver staining detection, and the obtained SIN-D1 concentrations were determined by comparison with the band densities of the DEN1 NS1 protein purchased from Native Antigen Company as the standard (SIN in FIG. 21C).

Using SIN-D1, seven rounds of ExSELEX (ExSELEX-4) with Ds-containing DNA libraries were performed, using the selection conditions summarized in Table E3.

TABLE E3
ExSELEX conditions targeting SIN-DEN1 NS1. ExSELEX (ExSELEX-4) targeting Singaporean DEN1-NS1 was performed, using
the prepared SIN-DEN1 NS1 recombinant protein and the clinical serums (PD1-4). To increase the stringency of the selection
conditions, human serum (HS) was added to the binding buffer (additives) and urea was added to the washing buffer in later rounds.

| | | | Target | | | | | | | | |
|---|---|---|---|---|---|---|---|---|---|---|---|
| | | | Recombinant | Clinical Serum | | | | | | | |
| | Selection | DNA | Protein [nM] | (PD1-4) | Volume | | Binding | | | Counter | PCR |
| Round | Method | [nM] | SIN DEN1-NS | [μl] | [ml] | Aditives | Buffer | Time(min) | Washing | Selection | Cycles |
| 1 | C | 500 | 2.5 | — | 0.8 | — | BB1 | 60 | WB1 × 3 | — | 20 |
| 2 | C | 200 | 2.5 | — | 0.3 | — | BB1 | 30 | WB1 × 5 | Pre | 20 |
| 3 | B | 50 | 2.5 | — | 0.4 | 0.1% BSA | BB2 | 30 | WB2 × 5 | Pre, Post | 22 |
| 4 | B | 10 | — | 20 | 0.4 | 0.1% BSA, | BB2 | 30 | WB2 × 6 | Pre, Post | 15 |
| | | | | | | 10% HS | | | | | |
| 5 | B | 10 | — | 10 | 0.4 | 0.1% BSA, | BB2 | 30 | WB2 (+3 M | Pre, Post | 28 |
| | | | | | | 10% HS | | | Urea) × 10 | | |
| 6 | B | 3 | 1 | — | 0.4 | 0.1% BSA, | BB2 | 30 | WB2 (+3 M | Pre, Past | 22 |
| | | | | | | 10% HS | | | Urea) × 10 | | |
| 7 | B | 3 | — | 5 | 0.4 | 0.1% BSA, | BB2 | 30 | WB2 (+3 M | Pre, Post | 28 |
| | | | | | | 10% HS | | | Urea) × 10 | Total | 155 |

Separation of DNA-Target Complexes (Method):
B: Sandwich (Capture with mAb#D06, in 96-well plates)
C: Complex immobilization (Dynabeads ™ His-Tag isolation & Pulldown)
Buffers:
BB1: 20 mM Tris-HCl pH7.5, 150 mM NaCl, 1 mM MgCl2, 2.7 mM KCl, 5 mM imidazol, 0.005% Nonidet-P40
BB2: 20 mM Tris-HCl pH7.5, 150 mM NaCl, 1 mM MgCl2, 2.7 mM KCl, 5 mM imidazol, 0.05% Tween20
WB1: 20 mM Tris-HCl pH7.5, 150 mM NaCl, 1mM MgCl2, 2.7 mM KCl, 0.005% Nonidet P-40
WB2: 20 mM Tris-HCl pH7.5, 150 mM NaCl, 1mM MgCl2, 2.7 mM KCl, 0.05% Tween20

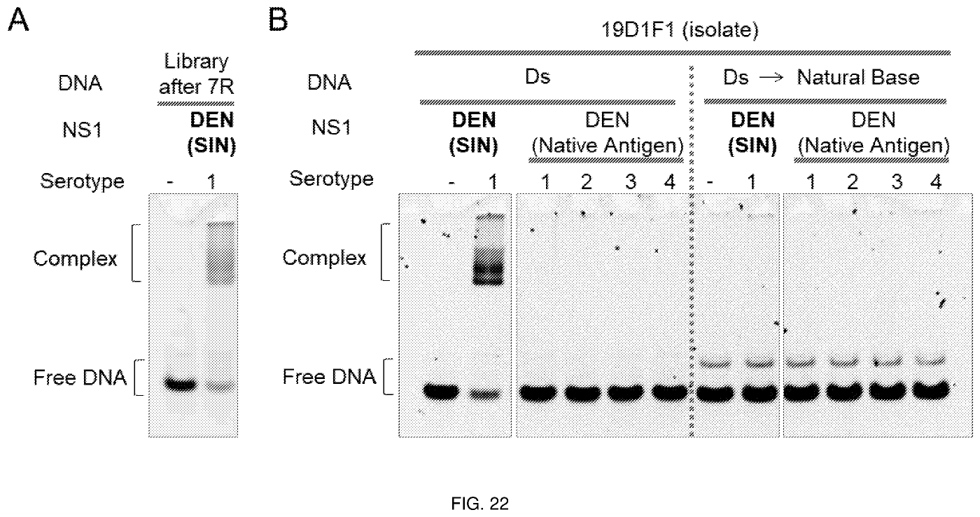

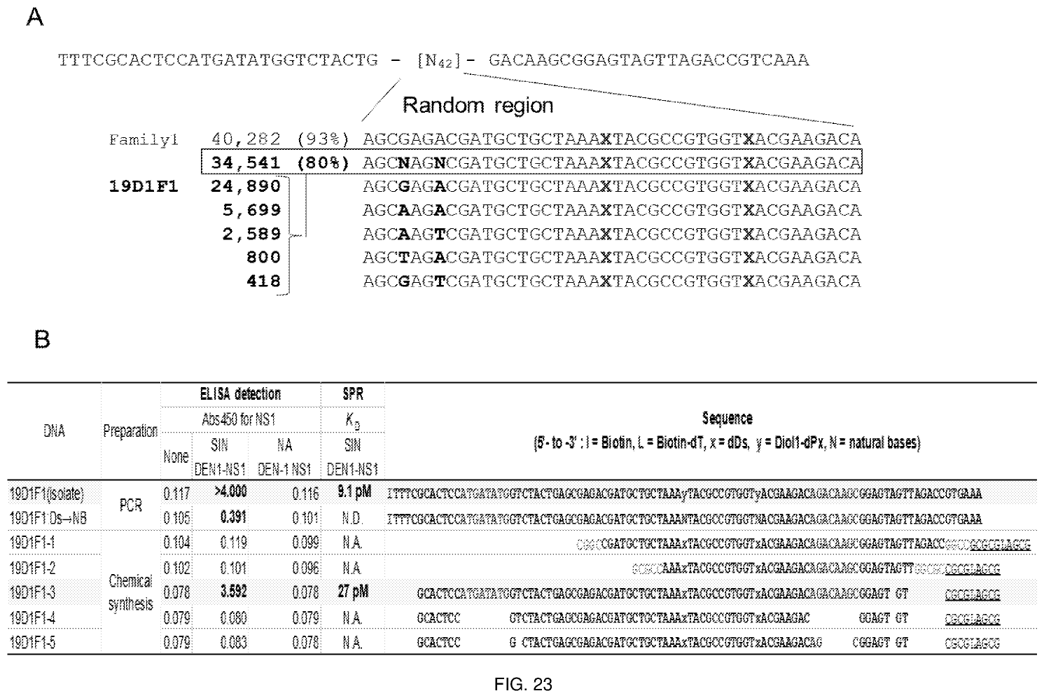

As the target, the prepared recombinant SIN DEN1-NS1 protein in rounds 1, 2, 3, and 6 were used, while the PD1-4 clinical serums in rounds 4, 5, and 7 were used. After seven rounds of ExSELEX, the binding of the enriched library to the SIN DEN1-NS1 protein in a gel-mobility shift assay (FIG. 22A) was observed. The sequences of the enriched library were then analyzed, and it was found that the library sequences (total extracted reads: 43,385) converged into a single family (Family 1, 40,282 reads) containing two Ds bases, with 93% of the population in the enriched library (FIG. 23A). When the sequence patterns in Family 1 were scrutinized, the 19D1F1 sequence was the most common (57% of the total extracted reads), and thus 19D1F1 was chosen for further characterization. The clone of 19D1F1 was isolated from the enriched library, using a biotinylated hybridization probe (5′-Biotin-CCACGGCGTATTTTAGCAGCATC).

The isolated 19D1F1 DNA was amplified by PCR in the presence or absence of the unnatural base substrates, dDsTP and dPxTP. The amplified 19D1F1 containing two Ds bases bound specifically to the SIN DEN1-NS1 protein. However, the Ds→NB (natural base) variant lost the binding ability (FIG. 22B), which indicates the importance of the Ds bases for the strong binding. In addition, it was confirmed that 19D1F1 only bound to the SIN DEN1-NS1 protein, and not to DEN1-NS1 or the other serotype NS1 proteins purchased from Native Antigen Company (FIG. 22B).

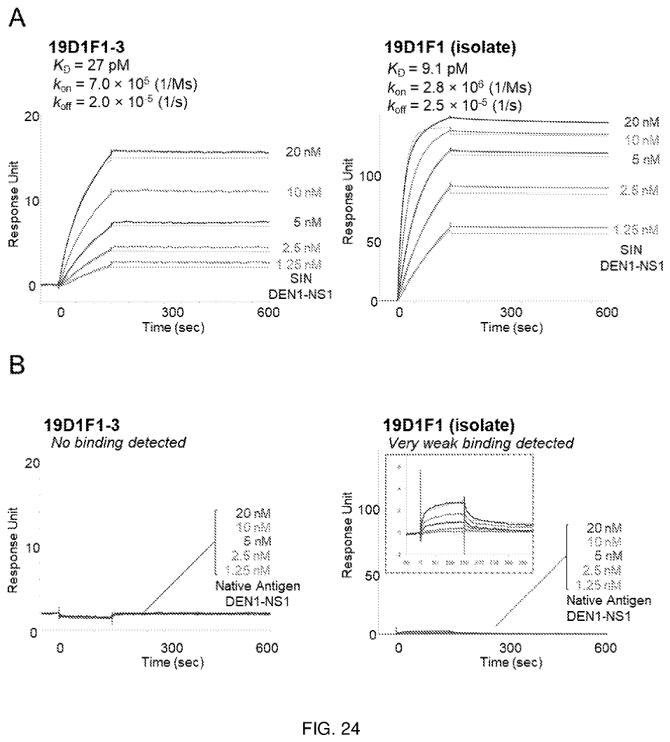

Five 19D1F1 derivatives, 19D1F1-1, 19D1F1-2, 19D1F1-3, 19D1F1-4, and 19D1F1-5 (FIG. 23B) were chemically synthesized. Among these derivatives, only 19D1F1-3 exhibited strong binding affinity to the SIN DEN1-NS1 protein (FIG. 24). The dissociation constants (K_{D}) of 19D1F1 UB-DNA aptamers, isolated from the enriched library (19D1F1 (isolate)) and chemically synthesized (19D1F1-3), were determined by SPR, and their K_{D} values were 9.1 pM and 27 pM, respectively.

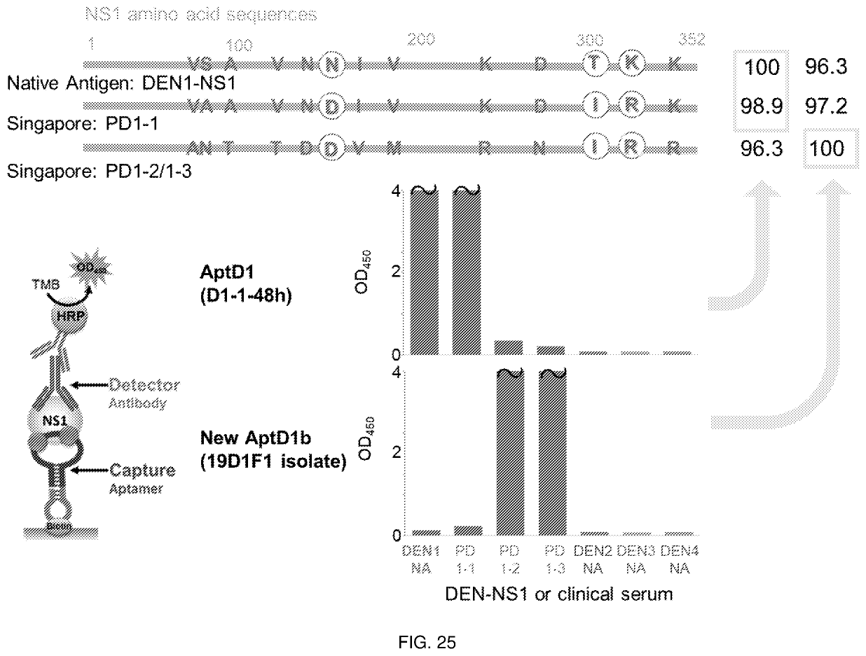

An ELISA with the sandwich system of the 19D1F1-3 (AptD1b) and an antibody pair was performed, using the clinical samples PD1-1, PD1-2 and PD1-3 (FIG. 25). The ELISA using the 19D1F1 isolate (AptD1b) efficiently and specifically detected PD1-2 and PD1-3, but not PD1-1 and the NS1 proteins (NA) of all serotypes. Therefore, the UB-DNA aptamers can recognize ˜97% amino acid homologies of target proteins beyond the serotype identification, which to knowledge are the highest specificities as ligands.

The sequences related to 19D1F1-3 are useful because the UB-DNA aptamers can be used for the detection of some variants of dengue serotype 1 NS1 proteins.

### Biological Experiments

### General Information for Biological Experiments and Materials

The DNA fragments, including DNA aptamer variants, DNA libraries, and primers, used in this study were chemically synthesized with an H8 DNA/RNA Synthesizer (K&A Laborgerate) in-house by using phosphoramidites, or purchased from Integrated DNA Technologies. The phosphoramidites of the natural bases were purchased from Glen Research, and the commercially available modified phosphoramidites were purchased from Glen Research, Link Technologies, and ChemGenes Corporation. The Ds and diolPa phosphoramidites were chemically synthesized in-house as described, previously³⁸ for Ds, and in the later chemical synthesis for diol-Pa. The chemically synthesized DNAs were purified by denaturing gel electrophoresis or directly used without further purification (for some primers and probes, which were purchased from IDT). Unnatural-base substrates (dDsTP, diol-dPxTP, Cy5-dPxTP, and dPa′TP) were chemically synthesized as described previously³⁸⁻⁴¹. Recombinant DEN-NS1 (DEN1-NS1, DEN2-NS1, DEN3-NS1, and DEN4-NS1 with a polyhistidine tag) were purchased from The Native Antigen Company (DEN1-NS: Nauru/Western Pacific/1974; DEN2-NS: Thailand/16681 /84; DEN3-NS1: Sri Lanka D3/H/IMTSSA-SRI/2000/1266; DEN4-NS1: Dominica/814669/1981). Recombinant Zika virus NS1 proteins (MR 766 Uganda strain and Brazil strain) were obtained from R&D Systems, Inc. and ACROBiosystems. Anti-dengue NS1 rabbit monoclonal antibodies were prepared in-house by the conventional method. Among the antibodies, Ab#D06 and Ab#D25, which had higher affinities than the others, were chosen. The streptavidin-HRP conjugate (1 mg/ml) was obtained from Jackson ImmunoResearch. The streptavidin, Tween 20, BSA, and anti-mouse IgG HRP conjugate (1 mg/ml) were obtained from Promega. General stock solutions and chemical compounds were purchased from Thermo Fisher Scientific, Nacalai Tesque, 1^{st} BASE, Promega, Sigma-Aldrich, New England Biolabs, and Bio-Rad Laboratories. The TMB-substrate solution (SureBlue Reserve™ TMB 1-Component Microwell Peroxidase Substrate, #5120-0083) was purchased from KPL. Control human serum was purchased from Sigma-Aldrich (Sigma #H4522) or obtained from healthy volunteers recruited at Tan Tock Sen Hospital (TTSH, Singapore), in a study approved by the National Healthcare Group Domain Specific Review Board (NHG DSRB) (Reference 2009/00432). Whole-blood samples were collected in a Serum Separation Transport Tube or EDTA tubes (Becton Dickinson) from dengue patients referred by the Communicable Disease Centre, TTSH. Blood specimens were obtained from patients consenting to the study. All patients provided separate written informed consent. The study protocol was approved by the NHG DSRB (reference 2015/00528 and 2016/00076). The recruited patients were tested and confirmed as NS1 positive from routine hospital diagnostics, using the SD BIOLINE Dengue Duo test, and had fever for less than 5 days from illness onset. They were confirmed to be infected by the dengue virus by an RTqPCR analysis of the samples. Dengue serotypes were also determined by an FTD dengue differentiation RT-qPCR test from Fast Track Diagnostics, using a Bio-Rad CFX96 instrument for the samples, and Sanger sequencing of RT-PCR products (as described later). The samples of a few patients that were followed up longitudinally were tested and samples at the acute phase (7 days post fever) and the convalescent phases (>7 days post fever up to 1 year) of their dengue infection were provided. In the ELISA shown below, serum samples were used for PD1-1, PD1-2, PD1-3, PD2-2, PD2-3, PD3-1, PD3-2, PD3-3, PD34, and PD4-1, while a plasma sample was used for PD2-1.

### ExSELEX.

In the first rounds of ExSELEX, two or four nmol of the single-stranded DNA libraries (88-mer, 5′-GCACTCCATGATATGGTCTACTG-N₄₂-GACAAGCGGAGTAGTTAGACCGT-3′) were used, which are a mixture of 74 sub-libraries. Each sub-library contains two Ds bases in the 42-nucleotide randomized sequence region, at predetermined positions. The ExSELEX conditions are summarized in Table E1. In general, the DNA library, diluted in binding buffer (20 mM Tris-HCl, pH 7.5, 150 mM NaCl, 1 mM MgCl₂, and 2.7 mM KCl), was denatured by heating at 95° C. for 5 min, immediately cooled on ice for 10 min, and then kept at room temperature (25° C.) for 10 min. To the diluted DNA library solution, Nonidet P-40 (Nacalai Tesque) or Tween 20 was added at the indicated concentrations. The library was incubated with each target protein (DEN1-NS1, DEN2-NS1, DEN3-NS1, or DEN4-NS1) at 25° C. in the presence or absence of additives (BSA and human serum). The DNA-NS1 complexes were separated from the unbound DNA species by using one of four different methods (Methods A-D), as shown in Table E1. Method A is ultrafiltration with Amicon Ultra Centrifugal Filter Units (MWCO: 100 kDa). Method B is capturing the complexes with an anti-DEN-NS1 antibody, Ab#D06, coated on microtiter plates (MaxiSorp™ 96-well plates from Thermo Fisher Scientific). Method C is a pull-down method, using Dynabeads His-Tag Isolation and Pulldown Magnetic Beads (Thermo Fisher Scientific). Method D is an electrophoresis gel-mobility shift assay⁴². In Methods A, B, and C, the captured DNA-NS1 complexes were washed several times, and the NS1-bound DNA was recovered by a treatment with 150 mM NaOH, followed by desalting with illustra MicroSpin G-25 Columns (GE Healthcare). The recovered DNA was amplified by PCR using forward 5′-PCR and reverse 3′-PCR primers, in the presence of unnatural substrates, dDsTP and diol-dPxTP^{42,43}. The reverse 3′-PCR primer contains a linker and spacer at the 5′-terminus, to differentiate the length of the library and its complementary strands, which allows their separation by denaturing polyacrylamide gel electrophoresis⁴⁴. The single-stranded Ds-DNA libraries were separated and purified by denaturing 8% PAGE, for the next round of selection. From Round 2, to remove the non-specific DNA binding species, pre-counter selections were performed, by incubating the DNA library solution with the magnetic beads only or in the antibody-coated wells on the plates, before the target binding. In ExSELEX-1 (Rounds 4-9) and ExSELEX-2 (Rounds 4-9), to remove the DNA species that bound to the other serotype NS1 proteins, post-counter selections were performed. In the post-counter selections, the DNA solutions, eluted from the DNA-NS1 complexes (before PCR), were incubated with the non-target serotype NS1 proteins at 25° C. for 30 min, and then the undesired DNA-NS1 complexes were removed from the solution with the magnetic beads. The resultant DNA solutions were subjected to PCR amplification.

### Deep Sequencing.

The aptamer candidate sequences were determined from the enriched DNA libraries in the final rounds of ExSELEX-1, ExSELEX-2, and ExSELEX-3, by the sequencing method with an Ion PGM system (Thermo Fisher Scientific), as described previously^{42,43,45}. The DNA libraries were amplified by replacement PCR without dDsTP, but with diol-dPxTP or dPa′TP⁴⁵. After the purification of the PCR products, the sequencing samples were prepared by using an Ion Plus Fragment Library Kit with an Ion Express Barcode Adapters 1-16 Kit and an Ion PGM Hi-Q View OT2 Kit, followed by deep sequencing using an Ion PGM Hi-Q View Sequencing Kit and Ion PGM 314 v2 chips (Thermo Fisher Scientific). The obtained sequence data were processed and clustered into families, and the unnatural base positions in the randomized region of each family were estimated by using in-house perl scripts.

### Identification of Px in the Aptamer Strand of 2D-1.

To identify the presence of diol-Px in the 2D-1 clones, the targeted family sequences were first captured from the enriched library in the final round of ExSELEX-3 targeting DEN2-NS1, by using a specific hybridization probe (5′-biotin-CCGCCTCTTGTTCCCAGTCGGAC-3′) (FIG. 8A). The DNA library (100 μl, 50 nM in probing buffer, 20 mM Tris-HCl, pH 7.6, 0.5 M NaCl, 10 mM MgCl₂) was annealed with the probe (1 μl, 5 μM in water), by heating at 95° C. for 3 min, followed by cooling by −0.1° C./sec to 60° C., and maintaining the solution at 60° C. for 15 min. The mixture was incubated with Hydrophilic Streptavidin Magnetic Beads (New England Biolabs) at 60° C. for 5 min. The magnetic beads, on which the target clones were hybridized with the probe, were then collected and washed five times with 150 μl of probing buffer (prewarmed at 60° C.). The hybridized clones were recovered from the beads by an incubation with 120 μl of water at 75° C. for 5 min. The recovered DNA (100 μl) was subjected to 20-cycle PCR (400 μl) in the presence of dDsTP and diol-dPxTP (50 μM each), and the aptamer strand was purified by denaturing PAGE. The binding of the isolated aptamer strand to DEN2-NS1 was confirmed by an electrophoresis gel-mobility shift assay (EMSA) (FIG. 8B). The chemically synthesized D2-1 DNA, D2-1-96(3Ds), in which three Ds bases were added at each position, did not bind to DEN2-NS1 (FIG. 8B), and thus, one of the three UB positions might be Px. The isolated aptamer strand (0.5 pmol) was amplified by 8-cycle PCR (25 μl), in the presence of 10 μM Cy5-dPxTP and 50 μM dDsTP with a FAM-labeled 5′-PCR primer and the linker-conjugated 3′-primer, to label the 5′-terminus of the aptamer strand with FAM and the Px-containing strand with Cy5³⁹. The PCR products were analyzed by denaturing 15% PAGE, and the band patterns were detected by the FAM and Cy5 fluorescence, with a bio-imaging analyzer, ChemiDoc™ MP (Bio-Rad) (FIG. 8C). By PCR using the linker-conjugated 3′-PCR primer, the mobility of the complementary strand of the aptamer sequence became slower than that of the aptamer strand, and both strands were identified separately on the gel (FIG. 8C). Both of the strands of the PCR products from the isolated D2-1 strand emitted Cy5 fluorescence (FIG. 8C), indicating that the D2-1 aptamer strand contains at least one Px base. The FAM-labeled aptamer strand in the remaining PCR product was purified by denaturing 8% PAGE for further experiments. Since the

Px nucleoside is degraded under basic conditions, the DNA fragments decompose at the Px position by a concentrated ammonia treatment at 55° C. for 4 hours. After removing the ammonia solution, the residue was suspended in 20 μl of Hi-Di Formamide (Thermo Fisher Scientific), and 10 μl aliquots were fractionated by denaturing 8% PAGE. The DNA band patterns on the gel were analyzed with a bio-imaging analyzer, LAS4000 (Fuji Film), before and after staining with SYBR Gold. From the digestion pattern on the gel, the Px position in D2-1 were assessed (FIG. 8D). The 5′-FAM fluorescence detection showed one band corresponding to the 5′-digested fragment (˜57-mer), and the detection by SYBR Gold staining showed two bands corresponding to the 5′-(˜57mer) and 3′-digested (˜39-mer) fragments. These digestion patterns indicated that the DNA fragment contained one Px base at the third unnatural base position (position 57) from the 5′ end (D2-1y-97 in Table E2).

### Preparation of the Authentic D2-1, D2-1y-96.

The authentic D2-1 aptamer, D2-1y-96, was prepared by using two chemically synthesized fragments (5-half: 5′-ACTCCATGATATGGTCTACTGGTCCG-Ds-CTGGGAACAAG-Ds-GGCGGGAGGGA-3′, 3-half: 5′-GGTCTAACTACTCCGCTTGTCGCACCCACACCC-Ds-TCCCTCCCGCC-3′, the complementary sequences are underlined), via primer extension and PCR amplification. The primer extension (100 μl) was performed by using 2 μM of each 5-half and 3-half in the presence of 50 μM dioldPxTP, followed by purification using a QIAquick Gel Extraction Kit (QIAGEN). By using the primer extension product as the template, 8-cycle PCR was performed in the presence of 50 μM each dDsTP and diol-dPxTP, and the aptamer strand was purified by denaturing 8% PAGE. The binding of the prepared aptamer strand, D2-1y-96, was analyzed by SPR and EMSA.

### Electrophoresis Gel-Mobility Shift Assays (EMSA).

For the DNA folding, the DNA fragments diluted in binding buffer were heated at 95° C. for 5 min, followed by immediate cooling on ice for 10 min. The DNA solution (50 nM) was mixed with or without the respective NS1 protein (25 nM) in binding buffer supplemented with 0.05% Nonidet P-40, and incubated at 25° C. for 30 min. After the incubation, the samples were mixed with glycerol (final concentration 5%), and the complex formation was analyzed by PAGE (4% polyacrylamide gel containing 44.5 mM Tris-Borate, 1 mM MgCl₂, 2.7 mM KCl, 5% glycerol, with or without 2 M urea). Gel electrophoresis was performed at 26-28° C. for 50 min, in the constant temperature mode (at a 3 W setting with a temperature probe set at 30° C.). The DNA band patterns on the gels were detected with the LAS4000 imager after staining with SYBR Gold. The band densities corresponding to free DNAs were quantified using the Multi Gauge software, to quantify the relative shifted ratios for comparing the degrees of complex formation.

### Surface Plasmon Resonance (SPR) Analysis.

Binding affinity profiles were obtained at 25° C. on a Biacore T200 (GE Healthcare), using running buffer (binding buffer supplemented with 0.05% Tween 20). For the immobilization of each ligand (aptamer variant), streptavidin-coated sensor chips were used and the biotinylated aptamer variant was immobilized on the flow cell, by injecting 0.5 nM of the ligand solution in running buffer, at a flow rate of 0.5 μl/min for 960 sec. For some of the Ds-DNA aptamers (D1 and D2 aptamer variants), it was found that the immobilization in the presence of NS1 gave reproducible target binding profiles, which would result from the aptamer immobilization at an appropriately separated distance, to ensure efficient binding with the multimeric NS1 proteins. Binding kinetic profiles were monitored by injecting at least five different concentrations of the analyte solutions (0.625 nM to 20 nM) for 150 sec (binding), at a flow rate of 30 μl/min. The analyte dissociation patterns were then recorded for 600 sec or 1,200 sec (for D1-1-48 h, D21d-72 h, D3-2-59 h, and D4-3-57 h). To regenerate the ligand on the flow cell surface, a denaturation solution (50 mM NaOH) was injected for 5 sec, and then the ligand was equilibrated in running buffer for 10 min. The kinetic parameters for the target binding, association rates (kₒₙ), dissociation rates (k_{off}), and dissociation constants (K_{D}=k_{off}/kₒₙ), were determined with the BIAevaluation software, version 3.0, by using the double-reference subtraction method and global curve fitting (more than twice at each concentration) to a 1:1 Langmuir model.

### ELISA Using Aptamer-Antibody Pair (Apt/Ab ELISA).

All incubations were performed at room temperature. Microtiter plates (Maxisorp™ 96-well plates from Thermo Fisher Scientific) were coated with 100 μl/well of 10 μg/ml streptavidin overnight, in 0.1 M sodium carbonate buffer (pH 9.6). The streptavidin-coated wells were blocked with 300 μl of 10 mg/ml BSA in 1×D-PBS (Nacalai Tesque) for two hours, and then the wells were washed three times with 200 μl of washing buffer (20 mM Tris-HCl, pH 7.5, 150 mM NaCl, 1 mM MgCl₂, 2.7 mM KCl, 0.05% Tween 20). Each UB-DNA aptamer was immobilized on the streptavidin-coated wells by a 2-hour incubation with 100 μl of 15 nM D1-1-48 h or 5 nM D2-1d-72 h, D3-3-59 h, or D4-3-57 h in dilution buffer (washing buffer supplemented with 1 mg/ml BSA), and then each well was washed three times with 200 μl of washing buffer. To the aptamer-coated wells, 100 μl of an NS1-Ab#D06 mixture solution was added and incubated for 30 min. The solutions were prepared beforehand at 1:9 ratios (vol/vol), by a 30-min incubation of each NS1 protein in dilution buffer or human serum with 11.1 nM of Ab#D06 in dilution buffer, supplemented with Tween 20 at a 2% final concentration (dilution buffer 2). After washing the wells once, 100 μl of secondary detector solution (anti-rabbit IgG HRP conjugate, diluted 1:2,500 with dilution buffer) was added to each well, and then incubated for 30 min. After washing the wells six times, 100 μl/well of TMB-substrate solution was added and incubated for 30 min. After adding 100 μl of 1 N HCl to the well to stop the reaction, the absorbance of the wells at 450 nm (OD₄₅₀) was measured with a microplate reader, Cytation 3 (BioTek). The assays under each condition were performed in duplicate (n=2), and the average absorbance data are shown in the graphs with error bars, which represent one standard deviation. When at least one of the two sample wells showed overflow (OD₄₅₀>4.000), the data are shown in the graphs with wavy lines.

### ELISA Using an Antibody-Antibody Pair (Ab/Ab ELISA).

The Ab/Ab ELISA was performed in a similar manner to the Apt/Ab ELISA, with some modifications. Instead of the aptamer-coated plates, the antibody-coated plates were prepared by a 2-hour incubation with 2 μg/ml Ab#D25 (100 μl/well) in 0.1 M sodium carbonate buffer (pH 9.6), followed by blocking with BSA. In the process to prepare the NS1-Ab#D06 mixture solutions with dilution buffer 2, biotinylated Ab#D6 was used. For biotinylation, the Ab#D25 solution (6.67 μM in 1×D-PBS) was mixed with Thermo Scientific™ EZ-Link™ Sulfo-NHS-LC-Biotin (final concentration 117 μM), and the mixture was incubated at room temperature for 30 min. The antibody was then recovered after desalting, using Amicon Ultra-0.5 Centrifugal Filter Units (MWCO: 50 kDa). The biotinylated Ab#D06 solution in 1×D-PBS was kept at 4° C. until use. The secondary detector used was a streptavidin-HRP conjugate, diluted 1:20,000 with dilution buffer, instead of the anti-rabbit IgG HRP conjugate.

Treatment of Control Human Serum with Protein A Resin

To remove the IgG from human serum, protein A resin was utilized. Human serum from Sigma (500 μL, Lot#SLBT0310) was incubated with Amintra Protein A Resin (Expedeon, 500 μl of a slurry, washed three times with 1 ml of dilution buffer) at room temperature for two hours with rotation. After the incubation, the resin was removed by centrifugation, and the supernatant was recovered and kept at 4° C. until use.

### Serology Testing and Dengue NS1 Detection

For control comparisons, anti-dengue IgG and IgM serology detection and dengue NS1 detection were performed using commercially available lateral flow assays, the Panbio Dengue Duo Cassette (Alere) and the SD BIOLINE Dengeu NS1 Ag rapid test (Alere) (FIG. 13), with acute phase samples. In the NS1 detection, 100 μl of each sample (human serum) was added to the sample well. After 20 min, the test and control lines were checked visually, with the naked eye. In the high-titer IgG and IgM detection, 10 μl of each sample (human serum) was dropped onto the sample well, and immediately two drops of the buffer included in the kit were added. After 15 min, the test lines for IgG and IgM, as well as the control line, were checked visually, with the naked eye. The presence of IgG indicates a secondary infection, whereas the absence of IgG indicates a primary infection.

### Competitive IgG Detection

For the assays with Apt/Ab ELISA, the wells coated with each UB-DNA aptamer as the capture agent were used. For the preparation of loading samples, a serum sample (5 μl, directly or diluted 10-, 25-, 50- or 100-fold with dilution buffer) was first mixed with 0.5 μl of each NS1 protein (DEN1-NS1: 350 pg, DEN2-NS1: 350 pg, DEN3-NS1: 450 pg, DEN4-NS1 200 pg). The solution was then mixed with 45 μl of 11.1 nM Ab#D06 in dilution buffer 2, incubated for 30 min, and then loaded into the aptamer-coated well (50 μl) and incubated for 30 min. The subsequent procedures were performed as described above for the Apt/Ab ELISA.

For the assays with the Ab/Ab ELISA, the wells coated with Ab#D25 (overnight) as the capture agent were used. For the preparation of loading samples, a serum sample (5 μL or diluted 10-, 25-, 50- or 100-fold with dilution buffer) was first mixed with 0.5 μl of each NS1 protein (DEN1-NS1: 400 pg, DEN2-NS1: 250 pg, DEN3-NS1: 400 pg, DEN4-NS1: 300 pg). The solution was mixed with 45 μl of 11.1 nM biotinylated Ab#D06 in dilution buffer 2. The subsequent procedures were performed as described above for the Ab/Ab ELISA.

From the plots of OD₄₅₀ against the volume of human serum used in the ELISA, the relative IgG activity was calculated through the normalization of the serum volume to lower the OD₄₅₀ to 1.0 (5/[the serum volume required for the OD₄₅₀ to be 1.0]) (FIG. 16).

### DNA Sequencing of the Dengue NS1 Region in RNA Samples.

To compare the amino acid sequences of NS1 in the clinical samples with those of the targeted NS1 in the aptamer generation, sequencing analyses of the DENV NS1 gene RT-PCR products were performed, using Sanger capillary sequencing (for PD1-2, PD1-3, PD2-1, PD2-2, PD2-3, PD3-1, PD3-2, PD3-3, PD34, and PD4-1) or multiplex PCR followed by deep sequencing (PD1-1), with some modifications of the published protocol⁴⁶. RNA from the clinical samples was reverse transcribed into cDNA using Superscript III RNase H(−) Reverse Transcriptase (Thermo Fisher Scientific) and specific primers or random hexamers.

The resulting cDNA was then used as the template for PCR amplification, using Taq DNA polymerase (New England Biolabs), AccuPrime Pfx DNA polymerase (Thermo Fisher Scientific), or Q5 HighFidelity DNA polymerase (New England Biolabs). After purification of the PCR products from the agarose gels or directly using a QIAquick gel extraction kit (Qiagen), the products were subjected to a cycle sequencing reaction with a BigDye™ Terminator v3.1 Cycle Sequencing Kit (Thermo Fisher Scientific) or deep sequencing with an Ion PGM system (Thermo Fisher Scientific), following the manufacturer's instructions. The capillary sequencing was performed on a 3500 Genetic Analyzer (Thermo Fisher Scientific), and the sequence reads were assembled manually. For PD1-1, the reads obtained with the Ion PGM system were mapped and analyzed, using PD1-2 as the reference sequence, with the CLC Genomics Workbench software (CLC bio).

### Chemical Synthesis

### General Information for Chemical Synthesis

All reagents and solvents were purchased from standard suppliers (Tokyo Chemical Industry Co., Ltd., Sigma-Aldrich, and Merck). Thin layer chromatography was performed using TLC silica gel 60 F254 (Merck). Compounds were visualized by UV shadowing or staining with a sulfuric acid-methanol solution. Nucleoside derivatives were purified on a Gilson HPLC system with a preparative C18 column (μBONDASPHERE, Waters, 19 mm×150 mm). ¹H NMR and ³¹P NMR spectra were recorded on a Bruker magnetic resonance spectrometer. CDCl₃ and DMSO-d₆ were used as the solvents.

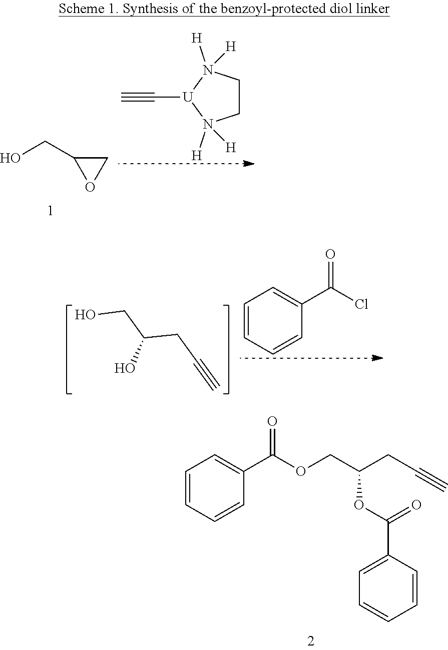

(S)-Pent-4-yne-1,2-diyl dibenzoate (2). Lithium acetylide ethylenediamine complex (8.31 g, 81.2 mmol) was dissolved in hexamethylphosphoric triamide (20 ml) and dry THF (80 ml), and the resulting mixture was cooled to 0° C. Afterwards, (R)-(+)-glycidol, compound 1, (1786 μl, 27 mmol) in dry THF (40 ml) was added dropwise with stirring at 0° C. The reaction mixture was stirred for 15.5 hours at ambient temperature, and then saturated NH₄Cl (200 ml) was added. The mixture was extracted with EtOAc (50 ml×3). The combined organic phase was dried over MgSO₄ and concentrated under reduced pressure. The residue was co-evaporated with dry pyridine twice. Benzoyl chloride (12.5 ml, 108 mmol) was added to the residue in dry pyridine (60 ml). The resulting mixture was stirred for 19 hours at ambient temperature. The reaction was quenched by the addition of methanol (10 ml) and stirred for 30 min at ambient temperature, prior to concentration under reduced pressure. EtOAc (150 ml) and water (150 ml) were poured into the resulting residue. The organic layer was separated and washed with water (150 ml), saturated aq-NaHCO₃ (150 ml), and brine (150 ml). The organic phase was dried over MgSO₄ and concentrated under reduced pressure. The resulting residue was purified by silica gel column chromatography (150 g of silica gel, hexane/EtOAc=100:0 to 95:5) to give compound 2 (2.86 g, 9.26 mmol, 34%). ¹H NMR (400 MHz, CDCl₃) δ 8.08-8.02 (m, 4H), 7.60-7.54 (m, 2H), 7.47-7.41 (m, 4H), 5.58-5.53 (m, 1H), 4.68 (dq, 2H, J=3.9, 12.0 Hz), 2.80 (dd, 2H, J=2.6, 6.2 Hz), 2.08 (t, 1H, J=2.6 Hz).

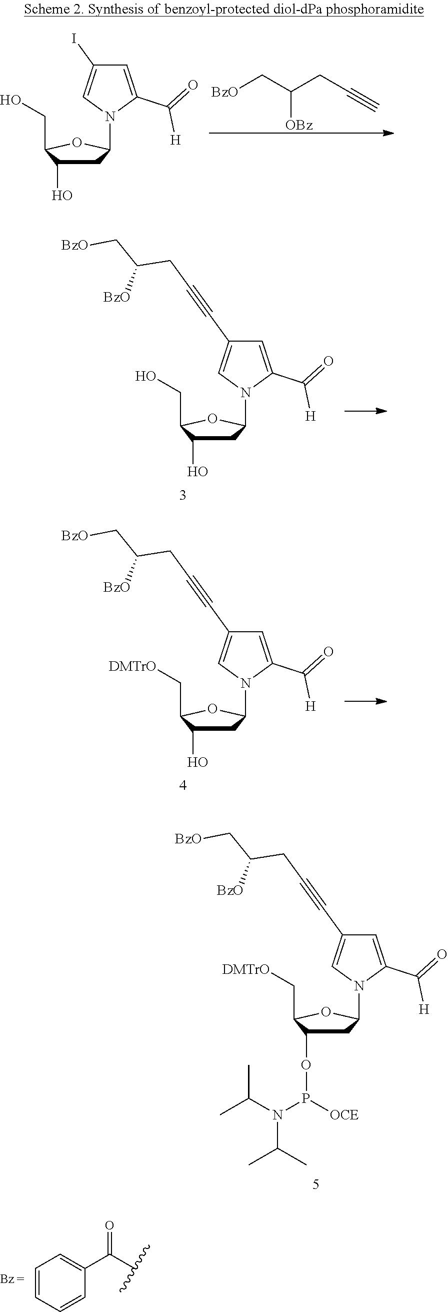

1-(2-Deoxy-β-D-ribofuranosyI)-(S)-4-(4,5-dibenzoyloxy-pent-1-yn-1-yl)-1H-pyrrole-2-carbaldehyde (3). A mixture of iodo-dPa (1.94 g, 5.75 mmol), copper iodide (175 mg, 0.92 mmol), tetrakis(triphenylphosphine)palladium(0) (332 mg, 0.288 mmol), triethylamine (1.6 ml, 11.5 mmol), and DMF (30 ml) was stirred and degassed for 10 min under reduced pressure and then flushed with argon. To this mixture was added compound 2 (2.22 g, 7.19 mmol), and the resulting mixture was further degassed for 10 min under reduced pressure and flushed with argon, prior to stirring for 4 hours at ambient temperature. The reaction mixture was concentrated under reduced pressure. The resulting dark liquid mixture was purified by silica gel column chromatography (60 g of silica gel, DCM/methanol=100:0 to 98:2) and C18 RP-HPLC (eluted by a gradient of CH₃CN (40-80%) in H₂O) to give compound 3 (2.35 g, 4.53 mmol, 79%). ¹H NMR (400 MHz, DMSO-d₆) δ 9.47 (d, 1H, J=0.9 Hz), 8.00-7.95 (m, 4H), 7.87 (s, 1H), 7.70-7.64 (m, 2H), 7.56-7.50 (m, 4H), 7.08 (d, 1H, J=1.8 Hz), 6.66 (t, 1H, J=6.3 Hz), 5.57-5.52 (m, 1H), 5.27 (d, 1H, J=4.1 Hz), 5.03 (t, 1H, J=5.3 Hz), 4.74-4.70 (dd, 1H, J=3.3, 11.9 Hz), 4.64-4.59 (dd, 1H, J=6.7, 12.0 Hz), 4.24 (m, 1H), 3.81 (dt, 1H, J=3.6, 4.0 Hz), 3.62-3.50 (m, 2H), 3.03 (d, 2H, J=6.4 Hz), 2.32-2.08 (m, 2H).

1-(5-O-DMTr-2-deoxyl-β-D-ribofuranosyl)-(S)-4-(4,5-dibenzoyloxy-pent-1-yn-1-yl)-1H-pyrrole-2carbaldehyde (4). Compound 3 (2.35 g, 4.53 mmol) was co-evaporated with dry pyridine three times. The residue in dry pyridine (40 ml) was mixed with 4,4′-dimethoxytrityl chloride (DMTrCl, 1.84 g, 5.44 mmol). The resulting mixture was stirred for 2 hours at ambient temperature, prior to concentration under reduced pressure. EtOAc (150 ml) and water (150 ml) was poured into the resulting residue. The organic layer was separated and washed with saturated aq-NaHCO₃ (150 ml×1) and brine (150 ml×1). After drying with MgSO₄, the solvent was evaporated under reduced pressure. The residue was purified by silica gel column chromatography (60 g of silica gel, hexane/EtOAc=100:0 to 70:30) to give compound 4 (2.98 g, 3.63 mmol, 80%). ¹H NMR (400 MHz, DMSO-d₆) δ 9.47 (d, 1H, J=0.8 Hz), 7.98-7.94 (m, 4H), 7.68-7.63 (m, 3H), 7.57-7.47 (m, 4H), 7.39-7.37 (m, 2H), 7.31-7.19 (m 6H), 7.11 (d, 1H, J=1.8 Hz), 6.89-6.87 (m, 4H), 6.67 (t, 1H, J=5.9 Hz), 5.54-5.49 (m, 1H), 5.36 (d, 1H, J=3.8 Hz), 4.71-4.67 (dd, 1H, J=3.3, 11.9 Hz), 4.60-4.55 (dd, 1H, J=6.7, 12.0 Hz), 4.26 (m, 1H), 3.97-3.93 (m, 1H), 3.73 (d, 6H, J=1.0 Hz), 3.22-3.18 (dd, 1H, J=5.8, 10.4 Hz), 3.14-3.11 (dd, 1H, J=3.1, 10.4 Hz), 2.99 (d, 2H, J=6.4 Hz), 2.36-2.18 (m, 2H).

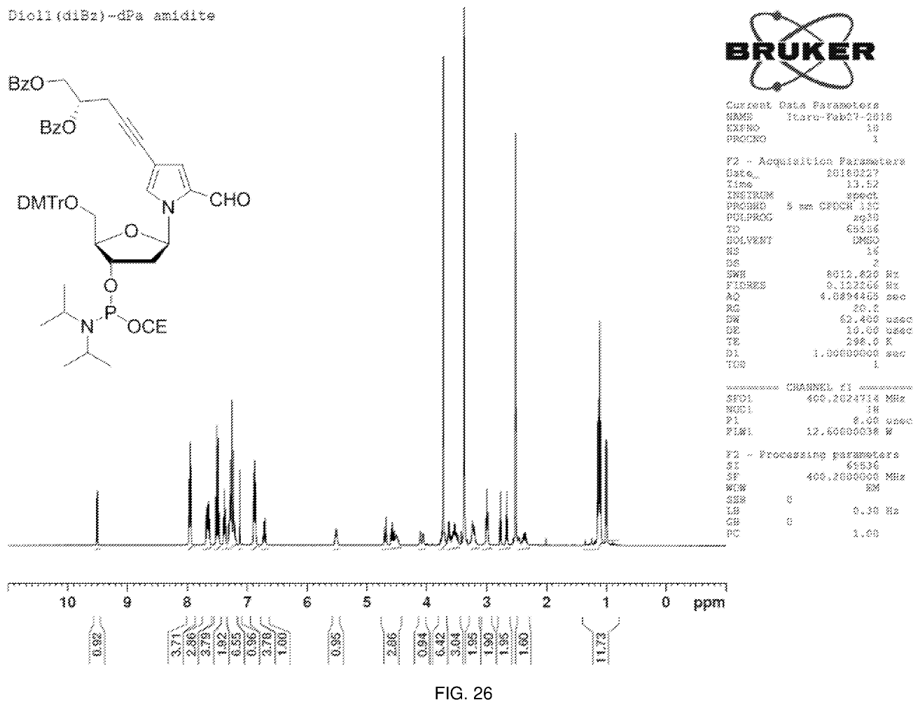

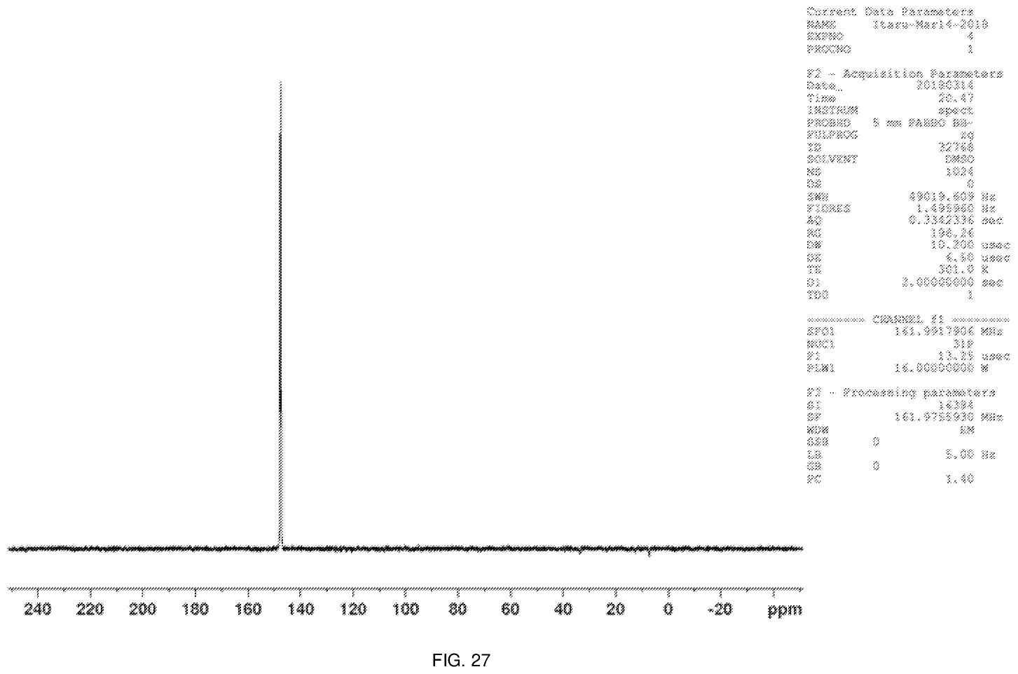

1-(5-O-DMTr-2-deoxyl3-D-ribofuranosyl)-(S)-4-(4,5-dibenzoyloxy-pent-1-yn-1-yl)-1H-pyrrole-2carbaldehyde phosphoramidite (5). Compound 4 (2.98 g, 3.63 mmol) was co-evaporated with pyridine three times and then with dry THF three times. N,N-Diisopropylethylamine (950 μl, 5.45 mmol) and 2cyanoethyl N,N-diisopropylchlorophosphoramidite (893 μl, 4 mmol) were added to the residue in anhydrous THF (35 ml), and the resulting mixture was stirred for 3 hours at ambient temperature. Dry methanol (500 μl) was added to the mixture to quench the reaction. EtOAc/triethylamine (150 ml, 99/1) and saturated aq-NaHCO₃ (150 ml) were poured into the resulting residue. The organic layer was separated and washed with aq-NaHCO₃ (150 ml) and brine (150 ml). After drying with MgSO₄, the solvent was evaporated under reduced pressure. The residue was purified by silica gel column chromatography (80 g, hexane/EtOAc=100/0 to 80/20 containing 1% triethylamine) to give compound 5 (2.84 g, 2.78 mmol, 76%). ¹H NMR (400 MHz, DMSO-d₆) δ 9.52-9.50 (m, 1H), 7.97-7.94 (m, 4H), 7.69-7.62 (m, 3H), 7.52-7.47 (m, 4H), 7.40-7.36 (m, 2H), 7.31-7.18 (m, 6H), 7.12 (m, 1H), 6.89-6.86 (m, 4H), 6.74-6.68 (m, 1H), 5.55-5.48 (m, 1H), 4.71-4.46 (m, 3H), 4.12-4.04 (m, 1H), 3.73-3.72 (m, 6H), 3.67-3.46 (m, 3H), 3.27-3.17 (m, 2H), 2.99 (t, 2H, J=5.9 Hz), 2.76 (t, 1H, J=5.9 Hz), 2.66 (t, 1H, J=5.9 Hz), 2.49-2.32 (m, 2H), 1.14-0.99 (m, 12H) (FIG. 26). ³¹P NMR (162 MHz, DMSO-d₆) δ 147.8 and 147.5 (diastereoisomers) (FIG. 27).

### REFERENCES

1 Bhatt, S. et al. The global distribution and burden of dengue. Nature 496, 504-507 (2013).
2 WHO. Dengue and severe dengue, <https://www.who.int/news-room/factHYPERLINK “https://www.who.int/news-room/fact-sheets/detail/dengue-and-severe-dengue”sheets/detail/dengue-and-severe-dengue>(2019).
3 Halstead, S. B. & O'Rourke, E. J. Dengue viruses and mononuclear phagocytes. I. Infection enhancement by non-neutralizing antibody. J. Exp. Med. 146, 201-217 (1977).
4 Halstead, S. B. & O'Rourke, E. J. Antibody-enhanced dengue virus infection in primate leukocytes. Nature 265, 739-741 (1977).
5 Halstead, S. B. In vivo enhancement of dengue virus infection in rhesus monkeys by passively transferred antibody. J. Infect. Dis. 140, 527-533 (1979).
6 Halstead, S. B. Dengue. Lancet 370, 1644-1652 (2007).
7 Guzman, M. G. & Harris, E. Dengue. Lancet 385, 453-465 (2015).
8 Guzman, M. G., Gubler, D. J., Izquierdo, A., Martinez, E. & Halstead, S. B. Dengue infection. Nat. Rev. Dis. Primers 2, 16055 (2016).
9 Wilder-Smith, A., Ooi, E. E., Horstick, O. & Wills, B. Dengue. Lancet 393, 350-363 (2019).
10 Priyamvada, L. et al. Human antibody responses after dengue virus infection are highly crossreactive to Zika virus. Proc. Natl. Acad. Sci. USA 113, 7852-7857 (2016).
11 Mathew, A. et al. B-cell responses during primary and secondary dengue virus infections in humans. J. Infect. Dis. 204, 1514-1522 (2011).
12 Corbett, K. S. et al. Preexisting neutralizing antibody responses distinguish clinically inapparent and apparent dengue virus infections in a Sri Lankan pediatric cohort. J. Infect. Dis. 211, 590-599 (2015).
13 Priyamvada, L. et al. B Cell Responses during Secondary Dengue Virus Infection Are Dominated by Highly Cross-Reactive, Memory-Derived Plasmablasts. J. Virol. 90, 5574-5585 (2016).
14 Katzelnick, L. C. et al. Antibody-dependent enhancement of severe dengue disease in humans. Science 358, 929-932 (2017).
15 St John, A. L. & Rathore, A. P. S. Adaptive immune responses to primary and secondary dengue virus infections. Nat. Rev. Immunol. 19, 218-230 (2019).
16 Patel, B. et al. Dissecting the human serum antibody response to secondary dengue virus infections. PLoS NegL Trop. Dis. 11, e0005554 (2017).
17 Reich, N. G. et al. Interactions between serotypes of dengue highlight epidemiological impact of cross-immunity. J. R. Soc. Interface 10, 20130414 (2013).
18 Villar, L. et al. Efficacy of a tetravalent dengue vaccine in children in Latin America. N. Engl. J. Med. 372, 113-123 (2015).
19 Capeding, M. R. et al. Clinical efficacy and safety of a novel tetravalent dengue vaccine in healthy children in Asia: a phase 3, randomised, observer-masked, placebo-controlled trial. Lancet 384, 1358-1365 (2014).
20 Ferguson, N. M. et al. Benefits and risks of the Sanofi-Pasteur dengue vaccine: Modeling optimal deployment. Science 353, 1033-1036 (2016).
21 Sridhar, S. et al. Effect of Dengue Serostatus on Dengue Vaccine Safety and Efficacy. N. Engl. J. Med. 379, 327-340 (2018).
22 Aguiar, M., Halstead, S. B. & Stollenwerk, N. Consider stopping dengvaxia administration without immunological screening. Expert. Rev. Vaccines 16, 301-302 (2017).
23 Halstead, S. B. Dengvaxia sensitizes seronegatives to vaccine enhanced disease regardless of age. Vaccine 35, 6355-6358 (2017).
24 Luo, R. et al. Rapid diagnostic tests for determining dengue serostatus: a systematic review and key informant interviews. Clin. Microbiol. Infect. (2019).
25 Bosch, I. et al. Rapid antigen tests for dengue virus serotypes and Zika virus in patient serum. Sci. Transl. Med. 9 (2017).
26 Lebani, K. et al. Isolation of serotype-specific antibodies against dengue virus non-structural protein 1 using phage display and application in a multiplexed serotyping assay. PLoS One 12, e0180669 (2017).
27 Ellington, A. D. & Szostak, J. W. In vitro selection of RNA molecules that bind specific ligands. Nature 346, 818-822 (1990).
28 Tuerk, C. & Gold, L. Systematic evolution of ligands by exponential enrichment: RNA ligands to bacteriophage T4 DNA polymerase. Science 249, 505-510 (1990).
29 Rothlisberger, P. & Hollenstein, M. Aptamer chemistry. Adv. Drug. Deliv. Rev. 134, 3-21 (2018).
30 Zhou, J. & Rossi, J. Aptamers as targeted therapeutics: current potential and challenges. Nat. Rev. Drug Discov. 16, 181-202 (2017).
31 Nimjee, S. M., White, R. R., Becker, R. C. & Sullenger, B. A. Aptamers as Therapeutics. Annu. Rev. Pharmacol. Toxicol. 57, 61-79 (2017).
32 McKeague, M. et al. Analysis of In Vitro Aptamer Selection Parameters. J. Mol. Evol. 81, 150161 (2015).
33 Kimoto, M., Yamashige, R., Matsunaga, K., Yokoyama, S. & Hirao, I. Generation of highaffinity DNA aptamers using an expanded genetic alphabet. Nat. Biotechnol. 31, 453-457 (2013).
34 Matsunaga, K., Kimoto, M. & Hirao, I. High-Affinity DNA Aptamer Generation Targeting von Willebrand Factor A1-Domain by Genetic Alphabet Expansion for Systematic Evolution of Ligands by Exponential Enrichment Using Two Types of Libraries Composed of Five Different Bases. J. Am. Chem. Soc. 139, 324-334 (2017).
35 Futami, K., Kimoto, M., Lim, Y. W. S. & Hirao, I. Genetic Alphabet Expansion Provides Versatile Specificities and Activities of Unnatural-Base DNA Aptamers Targeting Cancer Cells. Mol. Ther. Nucleic Acids 14, 158-170 (2019).
36 Sharma, M. et al. Magnitude and Functionality of the NS1-Specific Antibody Response Elicited by a Live-Attenuated Tetravalent Dengue Vaccine Candidate. J. Infect. Dis. (2019).
37 Halstead, S. B., Russell, P. K. & Brandt, W. E. NS1, Dengue's Dagger. J. Infect. Dis. (2019).
38 Hirao, I. et al. An unnatural hydrophobic base pair system: site-specific incorporation of nucleotide analogs into DNA and RNA. Nat. Methods 3, 729-735 (2006).
39 Yamashige, R. et al. Highly specific unnatural base pair systems as a third base pair for PCR amplification. Nucleic Acids Res. 40, 2793-2806 (2012).
40 Yamashige, R., Kimoto, M., Mitsui, T., Yokoyama, S. & Hirao, I. Monitoring the site-specific incorporation of dual fluorophore-quencher base analogues for target DNA detection by an unnatural base pair system. Org. Biomol. Chem. 9, 7504-7509 (2011).
41 Mitsui, T., Kimoto, M., Sato, A., Yokoyama, S. & Hirao, I. An unnatural hydrophobic base, 4propynylpyrrole-2-carbaldehyde, as an efficient pairing partner of 9-methylimidazo[(4,5)-b]pyridine. Bioorg. Med. Chem. Lett. 13, 4515-4518 (2003).
42 Matsunaga, K., Kimoto, M. & Hirao, I. High-Affinity DNA Aptamer Generation Targeting von Willebrand Factor A1-Domain by Genetic Alphabet Expansion for Systematic Evolution of Ligands by Exponential Enrichment Using Two Types of Libraries Composed of Five Different Bases. J. Am. Chem. Soc. 139, 324334 (2017).
43 Kimoto, M., Yamashige, R., Matsunaga, K., Yokoyama, S. & Hirao, I. Generation of high-affinity DNA aptamers using an expanded genetic alphabet. Nat. Biotechnol. 31, 453-457 (2013).
44 Kimoto, M., Matsunaga, K. I. & Hirao, I. Evolving Aptamers with Unnatural Base Pairs. Curr. Protoc. Chem. Biol. 9, 315-339 (2017).
45 Hamashima, K., Soong, Y. T., Matsunaga, K. I., Kimoto, M. & Hirao, I. DNA Sequencing Method Including Unnatural Bases for DNA Aptamer Generation by Genetic Alphabet Expansion. ACS Synth. Biol. (2019).
46 Quick, J. et al. Multiplex PCR method for MinION and Illumina sequencing of Zika and other virus genomes directly from clinical samples. Nat. Protoc. 12, 1261-1276 (2017).

### Applications

Embodiments of the aptamers are high-affinity and high-specificity unnatural-base (UB) DNA aptamers capable of binding to each serotype of dengue NS1 proteins. In some examples, embodiments of the aptamers have a hK_{D} of between from 30 pM to 182 pM. Embodiments of the aptamers can recognize target dengue NS1 proteins with amino-acid sequences that are more than 96.3% identical to that of the initial targets. Embodiments of the UB-DNA aptamers contain Ds (7-(2-thienyl)imidazo[4,5-b]pyridine) and/or diol-modified Pa (pyrrole-2-carbaldehyde) as a fifth and sixth base components.

Using embodiments of these UB-DNA aptamers, a simple and highly specific method to detect serotype-specific DENV infection is developed. In embodiments of the method, each serotype antigen of DEN-NS1 can be detected using UB-DNA aptamers that bind specifically to each DEN-NS1 serotype, by a sandwich-type ELISA format with an aptamer-antibody combination.

It was also found that anti-DEN-NS1 IgG in the patient's serum samples inhibit the aptamer's binding to the NS1 proteins. Further, from an analysis of sera from Singaporean patients with primary or secondary infection, it was further found that the IgG production initially reflected the serotype of the past infection, rather than that of the recent infection. Leveraging on these findings, a method to quantitatively identify the serotype-specific IgG antibodies to DEN-NS1 in serum was developed. In embodiments of the method, detection of serotype-specific IgG antibodies to dengue NS1 proteins was performed using a competitive ELISA format. In some examples, the detection of anti-DEN-NS1 IgG antibodies in a patient within one week after fever onset (e.g. during a febrile period) is indicative of a secondary infection in the patient, which may warrnt close monitoring.

Embodiments of the method trace serotype-specific dengue infection by detecting both viral NS1 proteins and their IgG antibodies in the early and later phase of dengue infection, by using ELISA with high-affinity DNA aptamers. Embodiments of the method allow the diagnosis of both past and current dengue infection, including serotype identification, and therefore facilitate early medical care and vaccine use decisions and analysis.

Embodiments of the method can potentially be expanded to test the efficacy in vaccine development, as well as the diagnoses of other diseases and allergies.

1. An aptamer for dengue virus (DENV), the aptamer comprising at least one unnatural base.

2. The aptamer according to claim 1, wherein the at least one unnatural base resides in a loop structure and/or a bulge of the aptamer.

3. The aptamer according to claim 1, wherein the at least one unnatural base is selected from the group consisting of:
7-(2thienyl)imidazo[4,5-b]pyridine (Ds);
7-(2,2′-bithien-5-yl)imidazo[4,5-b]pyridin-3-yl group (Dss);
pyrrole2-carbaldehyde (Pa);
2-nitro-4-propynylpyrrole (Px);
7-(2,2′,5′,2″-terthien-5-yl)imidazo[4,5-b]pyridin-3-yl group (Dsss);
2-amino-6-(2-thienyl)purin-9-yl group (s);
2-amino-6-(2,2′-bithien-5-yl)purin-9-yl group (ss);
2-amino-6-(2,2′,5′,2″-terthien-5-yl)purin-9-yl group (sss);
4-(2-thienyl)-pyrrolo[2,3-b]pyridin-1-yl group (dDsa);
4-(2,2′-bithien-5-yl)-pyrrolo[2,3-b]pyridin-1-yl group (Dsas);
4-[2-(2-thiazolyl)thien-5-yl]pyrrolo[2,3-b]pyridin-1-yl group (Dsav);
4-(2-thiazolyl)-pyrrolo[2,3-b]pyridin-1-yl group (dDva);
4-[5-(2-thienyl)thiazol-2-yl]pyrrolo[2,3-b]pyridin-1-yl group (Dvas);
4-(2-imidazolyl)-pyrrolo[2,3-b]pyridin-1-yl group (dDia);
derivatives thereof; and
combinations thereof.

4. The aptamer according to claim 1, wherein the aptamer comprises a DNA-based aptamer.

5. The aptamer according to claim 1, wherein the dissociation constant of the aptamer for DENV is no more than 200 pM.

6. The aptamer according to claim 1, wherein the aptamer is capable of binding to the NS1 protein of DENV.

7. The aptamer according to claim 1, wherein the aptamer is capable of binding specifically to a single serotype of DENV selected from the group consisting of serotype 1, serotype 2, serotype 3 and serotype 4.

8. The aptamer according to claim 1, wherein the aptamer comprises a sequence set out in the table below:

| | Sequence (L = Biotin-dT, x= dDs, |
|---|---|
| SEQ ID NO. | d = Diol1-dPa, y = Diol1-dPx, w = Diol1-dPa or Diol1-dPx) |
| 11 | CCCCAGACGGACTGGTGTxCTCGGxATGGCCGTCTGGGGCGCGLAGCG |
| 12 | GGCTGGTCCGxCTGGGAACAAGxGGCGGGAGGGAdGGGTGTGGGTGCGACAAGCGGA |
| | CCAGCCCGCGLAGCG |
| 13 | CCGCTTGTCATCTAxCCTGGCCxTGTGGTACTGTAACGGCTGACAAGCGGCGCGLAG |
| | CG |
| 14 | CGGCGGAGACGTAACGCxTATCAAATCxAAACAGCTTAGGGTCCGCCGCGCGLAGCG |
| 15 | Biotin-TTTCGCACTCCATGATATGGTCTACTGAGCGAGACGATGCTGCTAAAxTA |
| | CGCCGTGGTxACGAAGACAGACAAGCGGAGTAGTTAGACCGTGAAA |
| 16 | GCACTCCATGATATGGTCTACTGAGCGAGACGATGCTGCTAAAxTACGCCGTGGTxA |
| | CGAAGACAGACAAGCGGAGTGTCGCGLAGCG |
| 17 | LGATATGGTCTACTGTGTGAxGTCCTACAATGGACTGGTGTxCTCGGxATGGCCATT |
| | GACAAGCGGAGTAGTTAGACC |
| 18 | CAGACGGACTGGTGTxCTCGGxATGGCCGTCTGCGCGLAGCG |
| 19 | LTTTCGCACTCCATGATATGGTCTACTGGTCCGxCTGGGAACAAGxGGCGGGAGGGA |
| | dGGGTGTGGGTGCGACAAGCGGAGTAGTTAGACCGTCAAA |
| 20 | Biotin-TTTCGCACTCCATGATATGGTCTACTGGTCCGxCTGGGAACAAGxGGCGG |
| | GAGGGAyGGGTGTGGGTGCGACAAGCGGAGTAGTTAGACCGTCAAA |
| 21 | LTTTCGCACTCCATGATATGGTCTACTGGTCCGxCTGGGAACAAGxGGCGGGAGGGA |
| | dGGGTGTGGGTGCGACAAGCGGAGTAG |
| 22 | LCATGATATGGTCTACTGGTCCGxCTGGGAACAAGxGGCGGGAGGGAdGGGTGTGGG |
| | TGCGACAAGCGGAGTAG |
| 23 | GACGGTCTACTGGTCCGxCTGGGAACAAGxGGCGGGAGGGAdGGGTGTGGGTGCGAC |
| | AAGCGGAGTAGTTAGACCGTCCGCGLAGCG |
| 24 | GGTCTACTGGTCCGxCTGGGAACAAGxGGCGGGAGGGAdGGGTGTGGGTGCGACAAG |
| | CGGAGTAGACCCGCGLAGCG |
| 25 | GGTCCGxCTGGGAACAAGxGGCGGGAGGGAdGGGTGTGGGTGCGACAAGCGGCGCGL |
| | AGCG |
| 26 | LGATATGGTCTACTGAAGTGTTGTCATCTAxCCTGGCCxTGTGGTACTGTAACGGCT |
| | GACAAGCGGAGTAGTTAGACC |
| 27 | LGATATGGTCTACTGTGGCGCGAGGGAATCxACGCxTATCAAATAxAAACAGCTAAT |
| | GACAAGCGGAGTAGTTAGACC |
| 28 | LGATATGGTCTACTGAGGAGCGCATGTCGAGATACCAACCxCCATCCAATCxTTCTT |
| | GACAAGCGGAGTAGTTAGACC |
| 29 | LTGATATGGTCTACTGACGCCGGGGCCCGTAxTCAGACGTATACxCATCAGGGCACA |
| | TACAAGCGGAGTAGTTAGACC |
| 30 | CGAGGCCCGTAxTCAGACGTATACxCATCAGGGCCTCGCGCGLAGCG |
| 31 | GGCAGCGCGTCGATTGxCCAATCTTAGCCAACCCAAAATTACAAGCGCTGCCCGCGL |
| | AGCG |
| 32 | GCTGCCTxGTACCAACCCCCTCCAATCxATTAGGCAGCCGCGLAGCG |
| 33 | CGTGCGACGAxGTCCAACCAGTCCCAATCxACAAGTCGCACGCGCGLAGCG |
| 34 | GCGGTCCGTGCxGTCGCCAATCCGTGdTCCAACCCCGACAAGCGGACCGCCGCGLAG |
| | CG |
| 35 | GCCCGCTTTCGxCCAACCCGTGdTCCAATCCCAGAAAGCGGGCCGCGLAGCG |
| 36 | CGCCCGTCAAGGxCTCCAATCCGTGdTCCAACCAGTTTTGACGGGCGCGCGLAGCG |
| 37 | GCCCGCGTGCTCAACCTTACCAATCTGxCACGCGGGCCGCGLAGCG |
| 38 | GCCCTGCGxGCTCAACCTTACCAATCTGxCACGCAGGGCCGCGLAGCG |
| 39 | LACTCCATGATATGGTCTACTGATAGTACTCCxGTTTAACTCTGAxACTTGACGTCC |
| | ATTCATAGACAAGCGGAGTAGTTAGACC |
| 40 | LGATATGGTCTACTGGGGCTTGGTCTTGCGTxTGCAGATTAACTTGCGTGCCAGTAA |
| | GACAAGCGGAGTAGTTAGACC |
| 41 | LGATATGGTCTACTGTCTCAACGGTTGTCAAACGGxTATCACGGCxACACACCTGCG |
| | GACAAGCGGAGTAGTTAGACC |
| 42 | CTCCGCTGTCAAACGGxTATCACGGCxACACACCTGCGGACAGCGGAGCGCGLAGCG |
| 43 | LGATATGGTCTACTGTCACAxATCGCCGTAAAGxCGAAGAGCTGCGGAATCTAAGGT |
| | GACAAGCGGAGTAGTTAGACC |
| 44 | LGATATGGTCTACTGTATAATCCGCxTTCGTCATGTGGxTTGGATCTGGGTCTGGCA |
| | GACAAGCGGAGTAGTTAGACC |
| 45 | LGATATGGTCTACTGCCCAAxCTTGTCTGTAAGGGxTTGGxTAGGGCTGGCAAAAAA |
| | GACAAGCGGAGTAGTTAGACC |
| 46 | CGGCCGATGCTGCTAAAxTACGCCGTGGTxACGAAGACAGACAAGCGGAGTAGTTAG |
| | ACCGGCCGCGCGLAGCG |
| 47 | GCGCCAAAxTACGCCGTGGTxCGAAGACAGACAAGCGGAGTAGTTGGCGCCGCGLAG |
| | CG |
| 48 | GCACTCCGTCTACTGAGCGAGACGATGCTGCTAAAxTACGCCGTGGTxACGAAGACG |
| | GAGTGTCGCGLAGCG |
| 49 | GCACTCCGCTACTGAGCGAGACGATGCTGCTAAAxTACGCCGTGGTxACGAAGACAG |
| | CGGAGTGTCGCGLAGCG |
| 50 | GGCTGGTCCGACTGGGAACAAGxGGCGGGAGGGAdGGGTGTGGGTGCGACAAGCGGA |
| | CCAGCCCGCGLAGCG |
| 51 | ACTGGTGTxCTCGGxATGG |
| 52 | TGGGAACAAGxGGCGGGAGGGAwGGGTGTGGGTGCGACAAG |
| 53 | TCTAxCCTGGCCxTGTGGTACTGTAACGGC |
| 54 | GACGTAACGCxTATCAAATCxAAACAGCT |
| 55 | ATGATATGGTCTACTGAGCGAGACGATGCTGCTAAAxTACGCCGTGGTxACGAAGAC |
| | AGACAAGC |
| 56 | GAGGGAATCxACGCxTATCAAATAxAAACAGCT |
| 57 | AAACGGxTATCACGGCxACACACCTGCG |

or;
a sequence sharing at least 75% sequence identity thereto; or
a sequence differing by one, two, three, four, five, six, seven, eight, nine or ten bases thereto; or
portions thereof.

9. The aptamer according to claim 1 in combination with at least one, at least two or at least three other aptamers, wherein the mixture of aptamers are specific to different serotypes.

10. A method of identifying a DENV infection in a subject, the method comprising:
contacting a sample of the subject with the at least one, at least two, at least three or at least four of the aptamers according to claim 1, optionally wherein each aptamer is specific to a different serotype; and
detecting a binding event at the aptamer(s).

11. The method according to claim 10, wherein the method is a method of identifying a current DENV infection in the subject, and a binding event at any of the aptamer(s) is indicative of a current DENV infection in the subject,
optionally wherein the bound aptamer is specific to single DENV serotype and the binding event is indicative of a current DENV infection of said serotype in the subject.

12. The method according to claim 11, wherein where the subject is indicated for a current DENV infection, further comprising:
contacting a sample of the subject with at least one, at least two, at least three or at least four of the aptamers, optionally wherein each aptamer is specific to a different serotype; and
detecting a binding event at the aptamer(s),
wherein an absence of a binding event at any of the aptamer(s) is indicative that the current DENV infection is a secondary or further DENV infection,
optionally wherein the unbound aptamer(s) is specific to a DENV serotype and the absence of the binding event(s) is indicative of a past DENV infection of said serotype(s) in the subject.

13. The method according to claim 10, wherein the method is a method of identifying a past DENV infection in the subject, the contacting is performed in the presence of a DENV protein, and an absence of a binding event at any of the aptamer(s) is indicative of a past DENV infection in the subject,
optionally wherein the unbound aptamer(s) is specific to a DENV serotype and the absence of the binding event(s) is indicative of a past DENV infection of said serotype(s) in the subject.

14. The method according to claim 12, wherein the method comprises a competitive binding assay method.

15. The method according to claim 10, wherein the method is carried out within one week following fever onset in the subject.

16. The method according to claim 10, the method further comprising administering a DENV treatment regimen to the subject if the subject is indicated for a current DENV infection.

17. A method of evaluating a subject's suitability for a DENV vaccine, the method, comprising:
contacting a sample of the subject with at least one, at least two, at least three or at least four of the aptamers according to claim 1 in the presence of a DENV protein;
detecting a binding event at the aptamer(s);
determining an immune history of the subject based on the binding event at the aptamer(s), wherein an absence of a binding event at any of the aptamer(s) is indicative of a past DENV infection in the subject; and
concluding the suitability of the subject for the DENV vaccine based on the immune history.

18.-20. (canceled)

21. The method according to claim 13, wherein the method comprises a competitive binding assay method.

22. The aptamer according to claim 1 in combination with a DENV protein.
